(19) United States
(12) Patent Application Publication
(10) Pub. No.: US 2023/0416199 A1
(43) Pub. Date: Dec. 28, 2023

(54) INHIBITORS OF AMINO ACID TRANSPORT

(71) Applicants: ICAHN SCHOOL OF MEDICINE AT MOUNT SINAI, New York, NY (US); THE RESEARCH FOUNDATION FOR THE STATE UNIVERSITY OF NEW YORK, Albany, NY (US)

(72) Inventors: Christof GREWER, Vestal, NY (US); Avner SCHLESSINGER, New York, NY (US); Elias NDARU, Woburn, MA (US); Yueyue SHI, Binghamton, NY (US); Laura Jane ZIELEWICZ, Johnson City, NY (US)

(73) Assignees: ICAHN SCHOOL OF MEDICINE AT MOUNT SINAI, New York, NY (US); THE RESEARCH FOUNDATION FOR THE STATE UNIVERSITY OF NEW YORK, Albany, NY (US)

(21) Appl. No.: 18/249,142

(22) PCT Filed: Oct. 22, 2021
(86) PCT No.: PCT/US2021/071989

§ 371 Date: Apr. 14, 2023

Related U.S. Application Data
(60) Provisional application No. 63/104,661, filed on Oct. 23, 2020.

(51) Int. Cl. C07D 207/16; C07C 229/22

(52) U.S. Cl. CPC C07D 207/16; C07C 229/22

(57) ABSTRACT

Disclosed herein are compounds and compositions of formula I that are inhibitors of amino acid transport, specifically alanine serine cysteine transporter 2 (ASCT2):

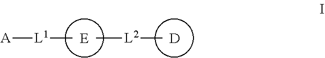

These compounds are useful as therapeutic agents for treating various cancers. Methods for inhibiting an ASCT2 transporter are also disclosed. Certain compounds are selective for an ASCT transporter.

### CROSS-REFERENCE TO RELATED APPLICATION

This application claims priority of U.S. provisional application 63/104,661, filed Oct. 23, 2020, the entire disclosure of which is hereby incorporated herein by reference.

### GOVERNMENT RIGHTS STATEMENT

This invention was made with government support under 1R01GM108911-01 awarded by the National Institutes of Health. The government has certain rights in the invention.

### FIELD OF THE INVENTION

The invention relates to compounds that are inhibitors of amino acid transport. In particular, compounds of the present invention selectively inhibit alanine serine cysteine transporter 2 (ASCT2). Compounds of the present invention are thus useful as therapeutic agents for treating various cancers.

### BACKGROUND OF THE INVENTION

The human SLC1 family is comprised of seven sodium-dependent amino acid transporters, including five excitatory amino acid transporters (EAAT1-5) and two alanine-serine-cysteine transporters (ASCT1 and ASCT2). The EAATs are primarily expressed in the central nervous system (CNS), where they mediate the uptake involved in the termination of glutamate neurotransmission. The ASCTs facilitate exchange of neutral amino acids in peripheral tissues such as the kidney, intestine, and skin and/or the CNS, and they are responsible for maintaining intracellular homeostasis of amino acids.

The Alanine-Serine-Cysteine Transporter 2 (ASCT2) is a sodium-dependent transporter of neutral amino acids. ASCT2 is expressed at low levels in various tissues including the intestine, kidney, liver, heart, placenta, and brain. ASCT2 belongs to the solute carrier 1 (SLC1) family, which includes glutamate transporters EAAT1-5 and neutral amino acid transporters ASCT1-2.

ASCT2 (SLC1A5) controls amino acid homeostasis in peripheral tissues. However, ASCT2 is highly upregulated in certain cancers, such as breast cancer, prostate cancer, and melanoma, and can correlate with poor prognosis, shown in studies of hepatocellular carcinoma and non-small cell lung cancer. ASCT2 modulates intracellular glutamine levels, thereby fueling cell proliferation, and in particular, ASCT2 imports glutamine into cells that is utilized to build biomass and enhance proliferation via mTORC1 (mammalian target of rapamycin complex 1), a process driven by the transcription factor c-MYC. Inhibition of ASCT2 has been shown to reduce intracellular glutamine levels and subsequently tumor size in vivo.

Small molecule inhibitors specific for ASCT2 are of value in treating diseases that arise from inappropriate ASCT2 activity and resulting cancer cell proliferation. Examples of such cancers include breast cancer, prostate cancer, and melanoma. Small molecule inhibitors that are specific for ASCT2 are also of value in biomedical research applications, such as diagnosing and/or visualizing brain tumors.

### SUMMARY OF THE INVENTION

Compounds have now been found that are potent inhibitors for ASCT2. Some compounds are selective for an ASCT transporter, in that they interact with the ASCT transporter with more potency than with an EAAT transporter.

In a first aspect, the invention relates to compounds of Formula I:

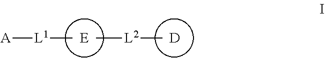

wherein:
A is selected from:

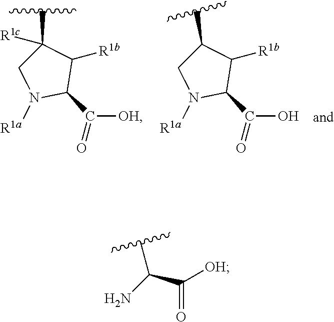

R^{1a} is selected from —H and —(C₁-C₆)alkyl;
R^{1b} is selected from —H, —OH, —CON(H)₂, and —NHC(═O)(H);
R^{1c} is —(CH₂)ₚOH—;
L¹ is selected from —(CHR²)ₙOR³—, —R³O(CHR²)ₙ—, —(CH₂)ₘN(R⁴)R³—, and —R³(R⁴)N(CH₂)ₘ—;
m is selected independently in each instance from 0 and 1;
n is selected from 0, 1, and 2;
p is selected from 0, 1, 2, and 3;
R² is selected independently in each instance from —H, —(CH₂)ₘOH, and —CH₃;
R³ is selected from —C(═O)— and —CH₂—;
R⁴ is selected independently in each instance from —H and —(C₁-C₆)alkyl;
L² is selected from a direct bond, —C(═O)—, —CH(OH)—, —NH(C═O)—, —(C═O)NH—, and —(CH₂)ₘO—(CH₂)ₘ—, or is absent when [symbol] is absent;
[symbol] is an aryl or heteroaryl moiety, optionally substituted with one or more substituents R⁵;
[symbol] is an aryl or heteroaryl ring, optionally substituted with one or more substituents R⁵, or is absent when [symbol] is a bicyclic or polycyclic moiety; and
R⁵ is selected independently in each instance from hydrogen, (C₁-C₆)alkyl, halogen, halo(C₁-C₆)alkyl, nitro, —OH, (C₁-C₆)alkoxy, halo(C₁-C₆)alkoxy, cyano, (C₁-C₆)alkylthio, halo(C₁-C₆)alkylthio, and amino.
   The person of skill will understand that both of R^{1c} and L¹ will not be connected to A by an oxygen atom or a nitrogen atom.

In a second aspect, the invention relates to a pharmaceutical composition comprising a pharmaceutically acceptable carrier and a compound described herein.

In a third aspect, the invention relates to a method for treating cancer in a patient in need thereof comprising administering an effective dose of a compound described herein.

In a fourth aspect, the invention relates to a method for treating a disease or disorder involving the dysregulation of ASCT2 in a patient. The method includes administering an effective amount of a compound described herein.

In a fifth aspect, the invention relates to a method for inhibiting ASCT2. The method includes bringing ASCT2 into contact with a compound described herein.

In a sixth aspect, the invention relates to a compound selective for an ASCT transporter, wherein a compound described herein interacts with the ASCT transporter, but interacts with less potency with an EAAT transporter.

### BRIEF DESCRIPTION OF THE DRAWINGS

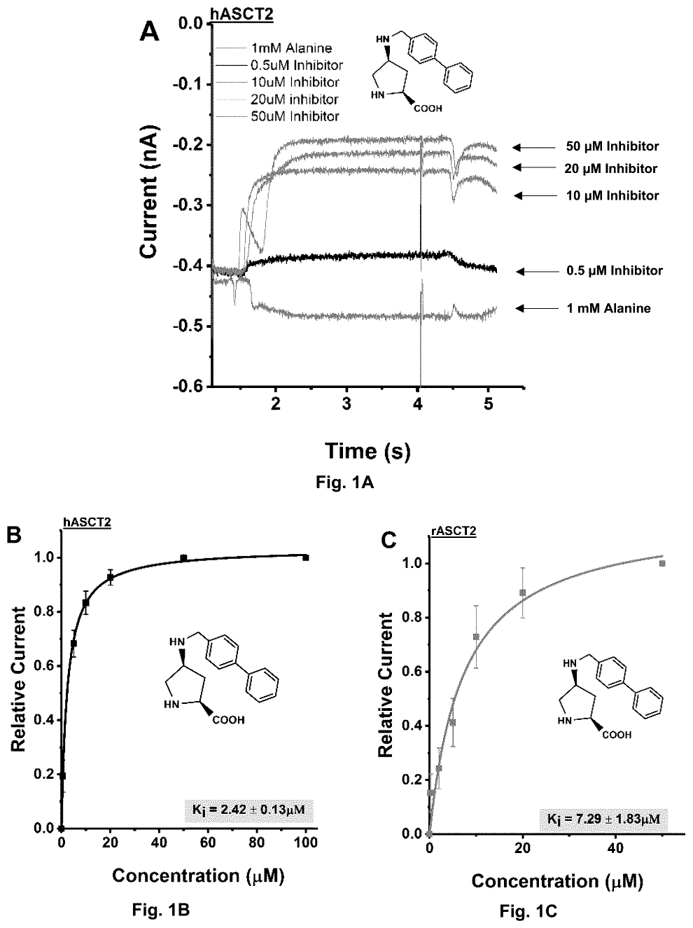

The figures show typical electrophysiological results used to obtain Kᵢ Values. FIG. 1A shows original current traces were obtained when increasing concentrations of a compound disclosed herein were applied to ASCT2 expressing HEK293 cells. FIG. 1B depicts hASCT2 dose-response curve fitted from current traces shown in FIG. 1A. FIG. 1C shows rASCT2 dose-response fit obtained from current traces similar to that shown in in FIG. 1A.

### DETAILED DESCRIPTION OF THE INVENTION

Changes in cell metabolism support rapid growth and proliferation of tumor cells, resulting in increased reliance on the metabolism of amino acids such as glutamine (i.e., ‘glutamine addiction’). Membrane transporters belonging to the Solute Carrier (SLC) families, such as the amino acid transporter ASCT2, are highly upregulated in multiple cancers, where they often function in cooperation with other mechanisms to supply tumor cells with nutrients that are used as energy supply, to build biomass, or to serve as signaling molecules to enhance cell proliferation. The novel compounds developed for this disclosure act as competitive inhibitors of the glutamine transporter ASCT2. Several of these inhibitors have sub-microM potencies, improving on published compounds with affinities in the 2-100 microM range. These inhibitors can block glutamine transport in rapidly growing cancer cells. These inhibitors can also serve as model compounds for basic science research of ASCTs in cell biology and metabolism; for instance, they can be used as biochemical tools to characterize glutamine transport by ASCT2 in cell models or in model organisms. They could also be potential pharmacological tools to block cancer cell growth.

Specific cancers in which ASCT2 has been established as a drug target include triple negative breast cancer, and prostate cancer. However, the inhibitors described herein are useful in treating any cancer in which ASCT2 is over-expressed. The ASCT2 inhibitors are also useful as pharmacological tools in transporter research. As of now, the prototypical small molecule inhibitors used for ASCT2 research in many publications are GPNA and benzylserine. However, these two compounds suffer from lack of potency and specificity. The best published compounds to date have Kᵢ values in the low microM range, which is not appropriate for in-vivo applications.

In a composition aspect, the invention relates to compounds of Formula I.

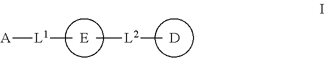

as described above.

In some embodiments, A is

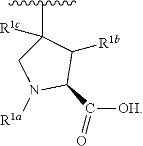

In other embodiments, A is

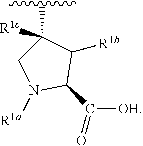

In still other embodiments, A is

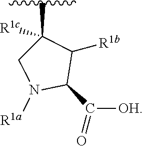

In some embodiments, A is

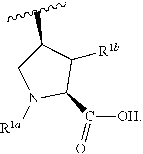

In other embodiments, A is

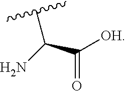 [SMILES: *[C@H](N)C(=O)O]

In some embodiments, A is

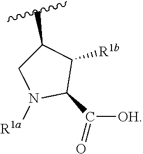

In some embodiments, R^{1a} is —H. In other embodiments, R^{1a} is —(C₁-C₆)alkyl. In some embodiments, R^{1a} is methyl.

In some embodiments, R^{1b} is —H. In other embodiments, R^{1b} is —OH. In still other embodiments, R^{1b} is CON(H)₂. In yet other embodiments, R^{1b} is —NHC(═O)(H);

In some embodiments, R^{1a} and R^{1b} are both —H.

In some embodiments, R^{1c} is —(CH₂)ₚOH—. In some embodiments, p is 0, and R^{1c} is —OH—. In other embodiments, p is 1 and R^{1c} is —(CH₂)OH—. In still other embodiments, p is 2. In yet other embodiments, p is 3.

In some embodiments, L¹ is —(CHR²)ₙOR³—. In other embodiments, L¹ is —OC(═O)—. In some embodiments, L¹ is —R³O(CHR²)ₙ—. In other embodiments, L¹ is —(CH₂)ₘN(R⁴)R³—. In still other embodiments, L¹ is —NHC(═O)—. In yet other embodiments, L¹ is —NH(CH₂)—. In some embodiments, L¹ is —R³(R⁴)N(CH₂)ₘ—.

In some embodiments, m is 0. In other embodiments, m is 1. It is to be understood that each instance of m is selected independently in each instance.

In some embodiments, n is 0. In other embodiments, n is 1. In still other embodiments, n is 2.

In some embodiments, R² is —H. In other embodiments, R² is —(CH₂)ₘOH. In other embodiments, R² is —OH. In yet other embodiments, R² is —CH₂OH. In still other embodiments, R² is —H or —OH. In some embodiments, R² is —CH₃. It is to be understood that each instance of R² is selected independently in each instance. As a non-limiting example, if L¹ is —(CHR²)ₙOR³— and n is 2, then L¹ could be —(CHOH)(CH₂)OR³—, that is, one instance of (CHR²) may be —CHOH—, while the other is —CH₂—.

In some embodiments, R³ is —C(═O)—. In other embodiments, R³ is —CH₂—.

In some embodiments, R⁴ is —H. In other embodiments, R⁴ is —(C₁-C₆)alkyl. In still other embodiments, R⁴ is methyl.

In some embodiments, L² is a direct bond. In other embodiments, L² is —C(═O)—. In yet other embodiments, L² is —CH(OH)—. In still other embodiments, L² is —NH(C═O)—. In some embodiments, L² is —(C═O)NH—. In other embodiments, L² is —(CH₂)ₘO—(CH₂)ₘ—. In still other embodiments, L² is —O—. In yet other embodiments, L² is —O—(CH₂)—. In other embodiments, L² is selected from a direct bond, —C(═O)—, —CH(OH)—, —NH(C═O)—, —O—, and —O—(CH₂)—. In still other embodiments, L² is selected from a direct bond and —NH(C═O)—. In some embodiments, L² is absent when [symbol] is absent.

In some embodiments, [symbol] is an aryl or heteroaryl moiety, optionally substituted with one or more substituents R⁵. In other embodiments, [symbol] is optionally substituted phenyl. In still other embodiments, [symbol] is optionally substituted thiophene. In yet other embodiments, [symbol] is optionally substituted pyridine. In some embodiments, [symbol] is optionally substituted anthracene. In other embodiments, [symbol] is optionally substituted pyrimidine. In some embodiments, [symbol] is optionally substituted furan. In yet other embodiments, [symbol] is optionally substituted indole. In still other embodiments, [symbol] is indole optionally substituted with methyl. In other embodiments, [symbol] is optionally substituted naphthalene. In still other embodiments, [symbol] is selected from optionally substituted phenyl, pyridine, and anthracene.

In some embodiments, [symbol] is an aryl or heteroaryl ring, optionally substituted with one or more substituents R⁵. In some embodiments, [symbol] is selected from optionally substituted phenyl, thiophene, pyridine, pyrimidine, and furan. In some embodiments, [symbol] is absent when [symbol] is a bicyclic or polycyclic moiety. In other embodiments, [symbol] is optionally substituted phenyl. In still other embodiments, [symbol] is optionally substituted thiophene. In yet other embodiments, [symbol] is optionally substituted pyridine. In other embodiments, [symbol] is optionally substituted pyrimidine. In some embodiments, [symbol] is optionally substituted furan.

In some embodiments, R⁵ is selected independently in each instance from hydrogen, (C₁-C₆)alkyl, halogen, halo(C₁-C₆)alkyl, nitro, —OH, (C₁-C₆)alkoxy, halo(C₁-C₆)alkoxy, cyano, (C₁-C₆)alkylthio, halo(C₁-C₆)alkylthio, and amino. In other embodiments, R⁵ is selected independently in each instance from hydrogen, (C₁-C₆)alkyl, halogen, halo(C₁-C₆)alkyl, nitro, —OH, and —(C₁-C₆)alkoxy. In still other embodiments, R⁵ is selected independently in each instance from hydrogen, methyl, fluoro, —CF₃, —CH₃Br, nitro, —OH, and methoxy. In yet other embodiments, R⁵ is hydrogen in each instance.

In some embodiments,

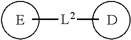

is selected from

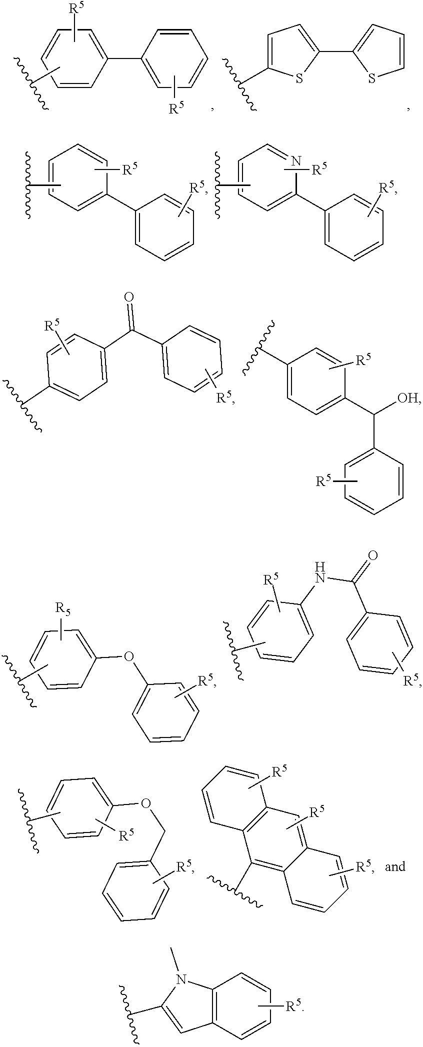

In other embodiments,

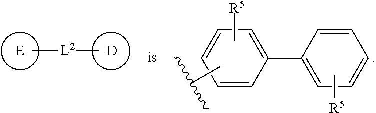

In still other embodiments,

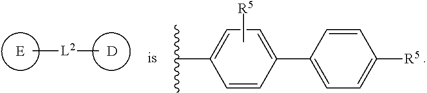

In yet other embodiments,

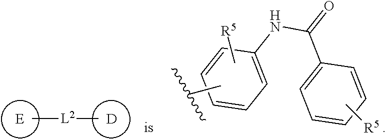

In some of these embodiments,

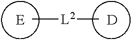

is selected from

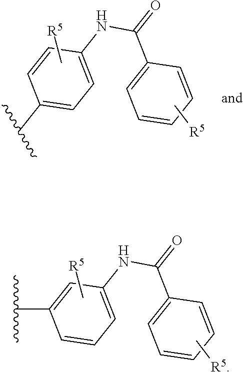

In some of these embodiments,

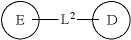

is selected from

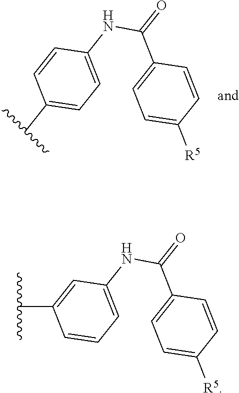

In some embodiments, the compound is a compound of formula IIa′:

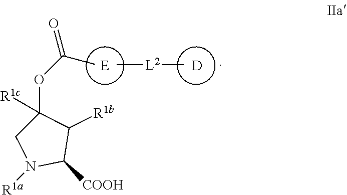

In some embodiments, the compound is a compound of formula IIa′¹:

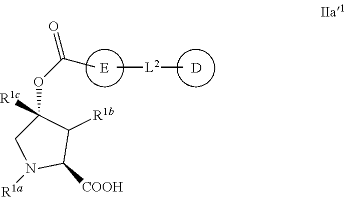

In some embodiments, the compound is a compound of formula IIa′²:

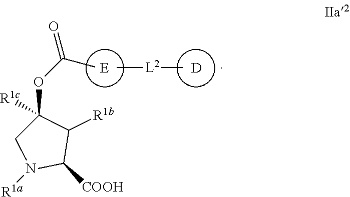

In some embodiments, the compound is a compound of formula IIa:

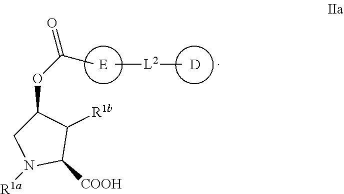

In some embodiments, the compound is a compound of formula IIb:

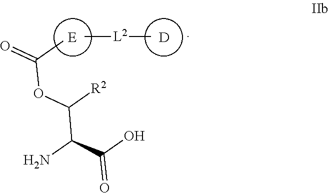

In some embodiments of formula IIa′, IIa′¹, IIa′², IIa, or IIb, R² is —H. In other embodiments of formula IIa′, IIa′, IIa′², IIa, or IIb, R² is —OH.

In some embodiments, the compound is a compound of formula IIIa′:

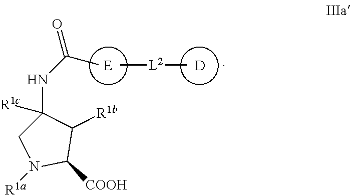

In some embodiments, the compound is a compound of formula IIIa′¹:

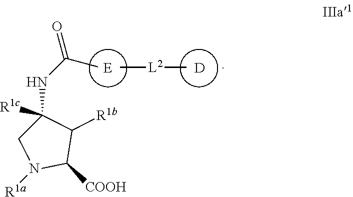

In some embodiments, the compound is a compound of formula IIIa′²:

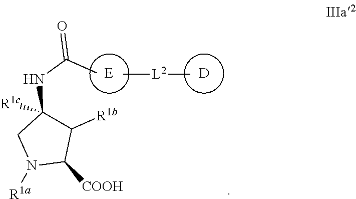

In some embodiments, the compound is a compound of formula Ilia:

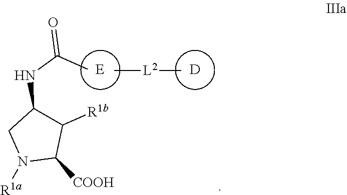

In some embodiments, the compound is a compound of formula IIIb:

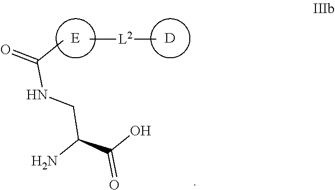

In some embodiments, the compound is a compound of formula IVa:′

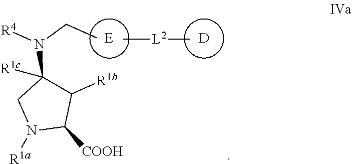

In some embodiments, the compound is a compound of formula IVa′¹:

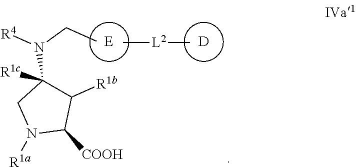

In some embodiments, the compound is a compound of formula IVa′²:

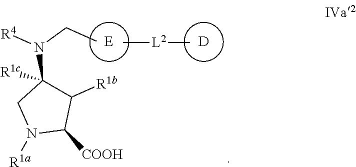

In some embodiments, the compound is a compound of formula IVa:

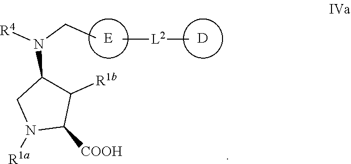

In some embodiments, the compound is a compound of formula Va′:

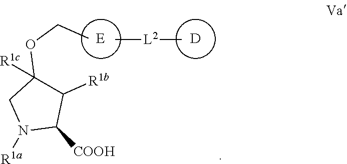

In some embodiments, the compound is a compound of formula Va′¹:

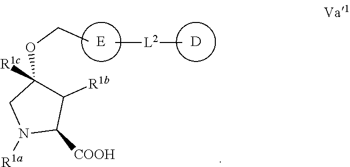

In some embodiments, the compound is a compound of formula Va′²:

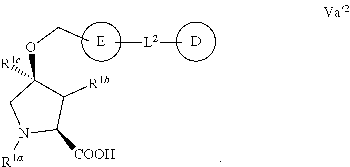

In some embodiments, the compound is a compound of formula Va:

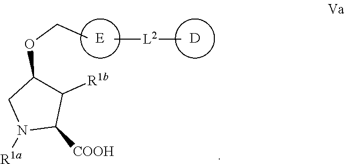

In some embodiments, the compound is a compound of formula Vb:

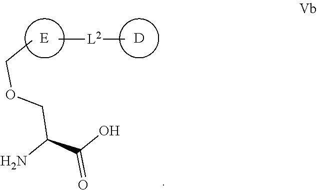

In some embodiments, R^{1a} and R^{1b} are both [symbol] , and both and [symbol] are optionally substituted phenyl.

One or more compounds described herein contain asymmetric centers and may thus give rise to enantiomers, diastereomers, and other stereoisomeric forms which may be defined in terms of absolute stereochemistry as (R)- or (S)-. The present invention is meant to include all such possible isomers as racemates, optically pure forms and intermediate mixtures. Optically active isomers may be prepared using homo-chiral synthons or homo-chiral reagents, or optically resolved using conventional techniques such as chiral chromatography. When the compounds described herein contain olefinic double bonds or other centers of geometric asymmetry, and unless specified otherwise, it is intended to include both (E)- and (Z)-geometric isomers. Likewise, all tautomeric forms are intended to be included.

The graphic representations of racemic, ambiscalemic and scalemic or enantiomerically pure compounds used herein are taken from Maehr J. Chem. Ed. 62, 114-120 (1985): simple, single bond lines convey connectivity only and no stereochemical implication; solid and broken wedges are used to denote the absolute configuration of a chiral element; wavy lines indicate explicit disavowal of any stereochemical implication which the bond it represents could generate; solid and broken bold lines are geometric descriptors indicating the relative configuration shown but do not denote absolute configurations; and wedge outlines and dotted or broken lines denote enantiomerically pure compounds of indeterminate absolute configuration.

For example, a generic structure depicting exemplary compounds of the invention is depicted as follows:

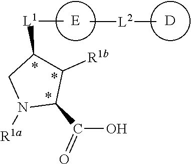

This structure contains three asymmetric (or potentially asymmetric) centers, labeled with asterisks (*). In one embodiment, this structure can be represented as:

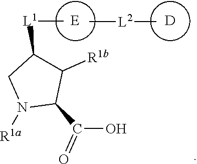

This depiction only indicates connectivity regarding the atoms bonded to the carbon attached to R^{1b}. The compound represented in this case could be a single stereoisomer or any mixture/combination of two possible stereoisomers, including a racemic mixture.

An exemplary individual stereoisomer is drawn, e.g., as follows:

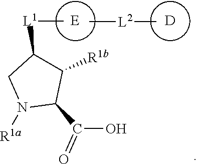

For a structure depicted using this convention, the absolute configuration is known to be as shown relative to the (S) α-carbon.

The graphic representation:

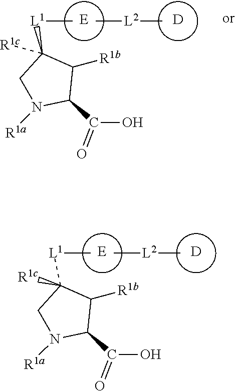

indicates a single enantiomer of absolute stereochemistry at the two position, but unknown absolute stereochemistry at the four position; that is, it could be either of

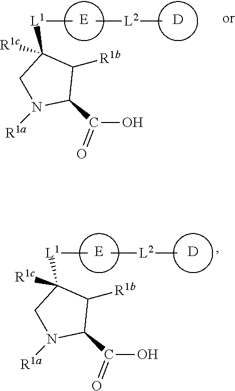

as a substantially pure single enantiomer.

In any of these possibilities, compounds can be a single stereoisomer or a mixture. If a mixture, the mixture will most commonly be racemic, but it need not be. Substantially pure single stereoisomers of biologically active compounds such as those described herein often exhibit advantages over their racemic mixture.

Enantiomerically pure means greater than 80 e.e., and preferably greater than 90 e.e. For the purpose of the present disclosure, a “pure” or “substantially pure” stereoisomer is intended to mean that the stereoisomer is at least 95% of the configuration shown and 5% or less of other stereoisomers, or at least 97% of the configuration shown and 3% or less of other stereoisomers, or at least 99% of the configuration shown and 1% or less of other stereoisomers.

Substituents are generally defined when introduced and retain that definition throughout the specification and in all independent claims.

The members of the genus described above exhibit biological activity in screens that are predictive of utility. However, it may be found upon examination that certain species and genera are not patentable to the inventors in this application. In this case, the exclusion of species and genera in applicants' claims are to be considered artifacts of patent prosecution and not reflective of the inventors' concept or description of their invention, which encompasses all members of the genus that are not in the public's possession.

As used herein, and as would be understood by the person of skill in the art, the recitation of “a compound”—unless expressly further limited—is intended to include salts of that compound. In a particular embodiment, the term “compound” refers to the compound or a pharmaceutically acceptable salt of that compound.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable examples of salts with inorganic bases include alkali metal salts such as sodium salts, potassium salts and the like; alkali earth metal salts such as calcium salts, magnesium salts and the like; aluminum salts; and ammonium salts. Preferable examples of salts with organic bases include salts with trimethylamine, triethylamine, pyridine, picoline, ethanolamine, diethanolamine, triethanolamine, dicyclohexylamine, N,N-dibenzylethylenediamine and the like.

Preferable examples of salts with inorganic acids include salts with hydrochloric acid, hydrobromic acid, nitric acid, sulfuric acid, phosphoric acid and the like. Preferable examples of salts with organic acids include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like. Preferable examples of salts with basic amino acids include salts with arginine, lysine, ornithine and the like. Preferable examples of salts with acidic amino acids include salts with aspartic acid, glutamic acid and the like.

Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic functionality (e.g. —SO₃H), suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed above, or a pharmaceutically acceptable salt form thereof, and a pharmaceutically acceptable carrier.

While it may be possible for the compounds of formula I to be administered as the raw chemical, it is preferable to present them as a pharmaceutical composition. According to a further aspect, the present invention provides a pharmaceutical composition comprising a compound of formula I or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

Formulations of the compounds and compositions described herein may be administered by a variety of methods: oral (including, but not limited to, capsules, cachets, tablets, powder, granules, solutions, suspensions, emulsions, tablets, or sublingual tablets), buccal, by inhalation (by using, for instance, an inhaler, a nebulizer, an aerosol, a gas, etc.), nasal, topical (including, but not limited to, lotions, creams, ointments, patches (i.e., transdermal), gels, liniments, pastes), ophthalmic, to the ear, rectal (for instance, by using a suppository or an enema), vaginal, or parenteral, depending on the severity and type of the disease being treated. In some embodiments, the compositions are administered orally or intravenously. The formulations include those suitable for oral, parenteral (including subcutaneous, intradermal, intramuscular, intracranial, intravenous and intraarticular), rectal, vaginal, nasal (inhalation), and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula I (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

It will be recognized that the compounds of this invention can exist in radiolabeled form, i.e., the compounds may contain one or more atoms containing an atomic mass or mass number different from the atomic mass or mass number usually found in nature. The compound of the present invention may be labeled with an isotope (e.g., ²H, ³H, ¹⁴C, ⁵S, ¹²⁵I, ¹¹C, ¹⁸F) and the like. Radioisotopes of hydrogen, carbon, phosphorous, fluorine, and chlorine include ²H, ³H, ¹³C, ¹⁴C, ¹⁵N, ³⁵S, ¹⁸F, and ³⁶Cl, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. The compound labeled with or substituted by an isotope can be used, for example, as a tracer used for Positron Emission Tomography (PET) (PET tracer) and is useful in the field of medical diagnosis and the like. Compounds that contain isotopes ¹¹C, ¹³N, ¹⁵O and ¹⁸F are well suited for PET. Radiolabeled compounds of formula I of this invention can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Compounds of the present invention can be used as a prophylactic or therapeutic agent for cancers such as breast cancer, prostate cancer, and melanoma. In general, compounds of the present invention can be used as a prophylactic or therapeutic agent for diseases involving the dysregulation of ASCT2.

In another aspect, the invention relates to a pharmaceutical composition comprising a pharmaceutically acceptable carrier and a compound as described herein.

In another aspect, the invention relates to a method or medicament for treating cancer in a patient in need thereof. The method includes administering an effective dose of a compound described herein. In some embodiments, the cancer is breast cancer. In other embodiments, the cancer is prostate cancer. In still other embodiments, the cancer is melanoma.

In another aspect, the invention relates to a method or medicament for inhibiting ASCT2. The method includes bringing ASCT2 into contact with a compound disclosed herein. In some embodiments, a compound disclosed herein is brought into contact with ASCT2 in vitro. In some embodiments, a compound disclosed herein is brought into contact with ASCT2 in vivo.

In another aspect, the invention relates to a method or medicament for treating a disease or disorder in a patient wherein the disease or disorder involves the dysregulation of ASCT2.

The method includes administering an effective dose of a compound described herein.

In another aspect, the invention relates to a compound selective for an ASCT transporter. In these aspects, a compound disclosed herein interacts with the ASCT transporter, but interacts with less potency with an EAAT transporter.

Various embodiments of the invention are described in the text below:

[1] A compound of formula I, wherein:

A is selected from:

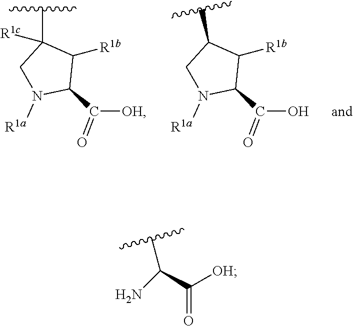

R^{1a} is selected from —H and —(C₁-C₆)alkyl;
R^{1b} is selected from —H, —OH, —CON(H)₂, and —NHC(═O)(H);
R^{1c} is —(CH₂)ₚOH—;
L¹ is selected from —(CHR²)ₙOR³—, —R³O(CHR²)ₙ—, —(CH₂)ₘN(R⁴)R³—, and —R³(R⁴)N(CH₂)ₘ—;
m is selected independently in each instance from 0 and 1;
n is selected from 0, 1, and 2;
p is selected from 0, 1, 2, and 3;
R² is selected independently in each instance from —H, —(CH₂)ₘOH, and —CH₃;
R³ is selected from —C(═O)— and —CH₂—;
R⁴ is selected independently in each instance from —H and —(C₁-C₆)alkyl;
L² is selected from a direct bond, —C(═O)—, —CH(OH)—, —NH(C═O)—, —(C═O)NH—, and —(CH₂)ₘO—(CH₂)ₘ—, or is absent when [symbol] is absent;
[symbol] is an aryl or heteroaryl moiety, optionally substituted with one or more substituents R⁵;
[symbol] is an aryl or heteroaryl ring, optionally substituted with one or more substituents R⁵, or is absent when [symbol] is a bicyclic or polycyclic moiety; and
R⁵ is selected independently in each instance from hydrogen, (C₁-C₆)alkyl, halogen, halo(C₁-C₆)alkyl, nitro, —OH, (C₁-C₆)alkoxy, halo(C₁-C₆)alkoxy, cyano, (C₁-C₆)alkylthio, halo(C₁-C₆)alkylthio, and amino.

[2] A compound according to [1] above, or according to other embodiments of the invention, wherein L¹ is —(CHR²)ₙOR³—.

[3] A compound according to [1] above, or according to other embodiments of the invention, wherein L¹ is —OC(═O)—.

[4] A compound according to [1] above, or according to other embodiments of the invention, wherein L¹ is —(CH₂)ₘN(R⁴)R³—.

[5] A compound according to [1] above, or according to other embodiments of the invention, wherein L¹ is —NHC(═O)—.

[6] A compound according to [1] above, or according to other embodiments of the invention, wherein L¹ is —NH(CH₂)—

[7] A compound according to any one of [1] to [6] above, or according to other embodiments of the invention, wherein [symbol] is selected from optionally substituted phenyl, thiophene, pyridine, anthracene, pyrimidine, furan, indole, and naphthalene.

[8] A compound according to any one of [1] to [7] above, or according to other embodiments of the invention, wherein [symbol] is selected from optionally substituted phenyl, thiophene, pyridine, pyrimidine and furan.

[9] A compound according to any one of [1] to [8] above, or according to other embodiments of the invention, wherein L² is selected from a direct bond, —C(═O)—, —CH(OH)—, —NH(C═O)—, —O—, and —O—(CH₂)—.

[10] A compound according to any one of [1] to [9] above, or according to other embodiments of the invention, wherein L² is selected from a direct bond and —NH(C═O)—.

[11] A compound according to any one of [1] to [6] above, or according to other embodiments of the invention, wherein

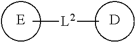

is selected from

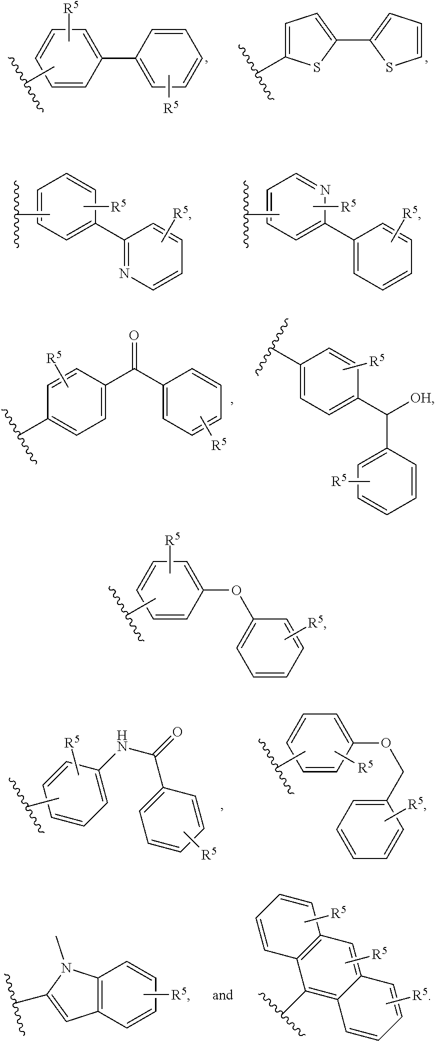

[12] A compound according to any one of [1] to [6] above, or according to other embodiments of the invention, wherein

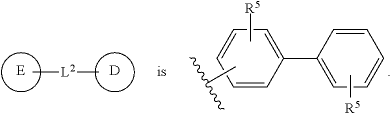

[13] A compound according to any one of [1] to [6] above, or according to other embodiments of the invention, wherein

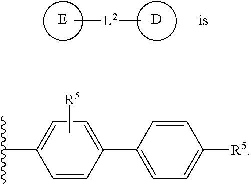

[14] A compound according to any one of [1] to [6] above, or according to other embodiments of the invention, wherein

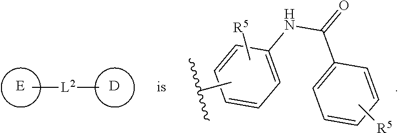

[15] A compound according to any one of [1] to [6] above, or according to other embodiments of the invention, wherein

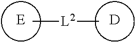

is selected from

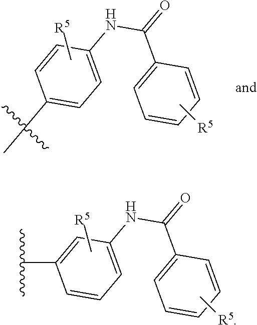

[16] A compound according to any one of [1] to [6] above, or according to other embodiments of the invention, wherein

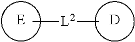

is selected from

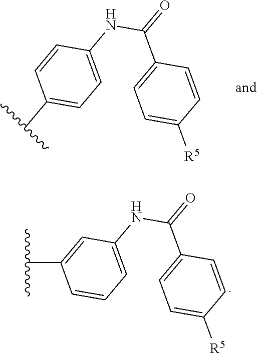

[17] A compound according to any one of [1] to [16] above, or according to other embodiments of the invention, wherein R⁵ is selected independently in each instance from hydrogen, (C₁-C₆)alkyl, halogen, halo(C₁-C₆)alkyl, nitro, —OH, and —(C₁-C₆)alkoxy.

[18] A compound according to any one of [1] to [17] above, or according to other embodiments of the invention, wherein R⁵ is selected independently in each instance from hydrogen, methyl, fluoro, —CF₃, —CH₃Br, nitro, —OH, and methoxy.

[19] A compound according to any one of [1] to [18] above, or according to other embodiments of the invention, wherein R⁵ is hydrogen in each instance.

[20] A compound according to any one of [1] to [10] or [17] to [19] above, or according to other embodiments of the invention, wherein [symbol] is selected from optionally substituted phenyl, pyridine, and anthracene.

[21] A compound according to any one of [1] to [20] above, or according to other embodiments of the invention, wherein A is

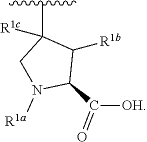

[22] A compound according to any one of [1] to [21] above, or according to other embodiments of the invention, wherein A is

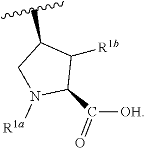

[23] A compound according to any one of [1] to [22] above, or according to other embodiments of the invention, wherein R^{1a} and R^{1b} are both —H.

[24] A compound according to any one of [1] to [21] or [23] above, or according to other embodiments of the invention, wherein R^{1c} is —OH or —(CH₂)OH.

[25] A compound according to any one of [1] to [20] above, or according to other embodiments of the invention, wherein A is

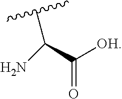 [SMILES: *[C@H](N)C(=O)O]

[26] A compound according to any one of [1] or [7] to [25] above, or according to other embodiments of the invention, wherein the compound is of formula IIa′, IIa′¹, IIa′², IIa or IIb.

[27] A compound according to [26] above, or according to other embodiments of the invention, wherein R² is —H or —OH.

[28] A compound according to any one of [1] or [7] to [25] above, or according to other embodiments of the invention, wherein the compound is of formula IIIa′, IIIa′¹, IIIa′², IIIa or IIIb.

[29] A compound according to any one of [1] or [7] to [25] above, or according to other embodiments of the invention, wherein the compound is of formula IVa′, IVa′¹, IVa′², or IVa.

[30] A compound according to any one of [1] or [7] to [25] above, or according to other embodiments of the invention, wherein the compound is of formula Va′, Va′¹, Va′², Va, or Vb.

[31] A compound according to any one of [1] to [30] above, or according to other embodiments of the invention, wherein the compound is selected from a compound shown in Table 1.

[32] A compound according to any one of [1] to [30] above, or according to other embodiments of the invention, wherein the compound is selected from a compound shown in Table 3.

[33] A pharmaceutical composition comprising a pharmaceutically acceptable carrier and a compound according to any one of [1] to [32] above, or according to other embodiments of the invention.

[34] A method for treating cancer in a patient in need thereof comprising administering an effective dose of a compound according to any one of [1] to [32] above, or according to other embodiments of the invention.

[35] A method for treating cancer selected from breast cancer, prostate cancer, and melanoma in a patient in need thereof comprising administering an effective dose of a compound according to any one of [1] to [32] above, or according to other embodiments of the invention.

[36] A method for treating a disease or disorder in a patient wherein the disease or disorder involves the dysregulation of ASCT2, the method comprising administering to the patient a therapeutically effective amount of a compound according to any one of [1] to [32] above, or according to other embodiments of the invention.

[37] A method for inhibiting ASCT2, wherein the method includes bringing ASCT2 into contact with a compound according to any one of [1] to [32] above, or according to other embodiments of the invention.

[38] An in vitro method for treating cancer in a patient in need thereof comprising administering an effective dose of a compound according to any one of [1] to [32] above, or according to other embodiments of the invention.

[39] An in vivo method for treating cancer in a patient in need thereof comprising administering an effective dose of a compound according to any one of [1] to [32] above, or according to other embodiments of the invention.

[40] A compound selective for an ASCT transporter, wherein the compound interacts with the ASCT transporter, but interacts with less potency with an EAAT transporter, wherein the compound is a compound according to any one of [1] to [32] above, or according to other embodiments of the invention.

### Abbreviations and Definitions

Unless defined otherwise, all technical and scientific terms used herein have the same meaning as is commonly understood by one of ordinary skill in the art to which this disclosure belongs. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

The following abbreviations and terms have the indicated meanings throughout:
Ac=acetyl
Aq=aqueous
Boc=t-butyloxy carbonyl
Bu=butyl
c-=cyclo
DCC=N,N′-dicyclohexylcarbodiimide
DCM=dichloromethane=methylene chloride=CH₂Cl₂
DIPEA or DIEA=N,N-Diisopropylethylamine
DMAP=4-N,N-dimethylaminopyridine
DMF=N,N-dimethylformamide
DTT=dithiothreitol
eq. or equiv.=equivalent(s)
Et=ethyl
mCPBA=meta-Chloroperoxybenzoic acid
Me=methyl
min.=minute(s)
Ph=phenyl
RT or rt=room temperature
sat'd or sat.=saturated
t- or tert=tertiary
TBAF=tetrabutylammonium fluoride
TBS=tert-butylmethylsilyl
TFA=trifluoroacetic acid
THE=tetrahydrofuran
tosyl=p-toluenesulfonyl

Throughout this specification the terms and substituents retain their definitions. The description provided herein uses certain terms known in the chemical arts. Unless otherwise specified throughout the description herein, terms retain their meaning as understood by one having ordinary skill in the art.

As used herein, the terms “comprising” and “including” or grammatical variants thereof are to be taken as specifying the stated features, integers, steps or components, but do not preclude the addition of one or more additional features, integers, steps, components or groups thereof. This term encompasses the terms “consisting of” and “consisting essentially of.”

The phrase “consisting essentially of” or grammatical variants thereof, when used herein, is to be taken as specifying the stated features, integers, steps or components, but does not preclude the addition of one or more additional features, integers, steps, components or groups thereof, but only if the additional features, integers, steps, components or groups thereof do not materially alter the basic and novel characteristics of the claimed composition or method.

As used herein, the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method that “comprises”, “has”, “includes” or “contains” one or more steps or elements possesses those one or more steps or elements, but is not limited to possessing only those one or more steps or elements.

A “patient” (or “subject”) as used herein, includes both humans and other animals, particularly mammals (e.g., human, mouse, rat, rabbit, dog, cat, bovine, horse, swine, monkey). Thus, the methods are applicable to both human therapy and veterinary applications. In some embodiments, the patient is a mammal, for example, a primate. In some embodiments, the patient is a human.

As used herein, the terms “treatment” or “treating” are used interchangeably. These terms refer to an approach for obtaining beneficial or desired results including, but not limited to, therapeutic benefit. Therapeutic benefit includes eradication and/or amelioration of the underlying disorder being treated; it also includes the eradication and/or amelioration of one or more of the symptoms associated with the underlying disorder such that an improvement is observed in the subject, notwithstanding that the subject may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life). A therapeutic benefit is achieved with the eradication or amelioration of one or more of the physiological systems associated with the underlying disorder such that an improvement is observed in the patient, notwithstanding that the patient may still be afflicted with the underlying disorder.

Treatment can involve administering a compound described herein to a patient diagnosed with a disease, and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The terms “administer”, “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of the compounds described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder.

Unless otherwise specified, alkyl (or alkylene, when divalent) is intended to include linear or branched saturated hydrocarbon structures and combinations thereof. Unless otherwise specified, “alkyl” refers to alkyl groups from 1 to 20 carbon atoms, or 1 to 10 carbon atoms, or 1 to 6 carbon atoms, or 1 to 4 carbon atoms, or 1 to 3 carbon atoms. Examples of alkyl groups include methyl, ethyl, propyl, isopropyl, n-butyl, s-butyl, t-butyl and the like.

Aryl means (i) a phenyl group (or benzene); (ii) a bicyclic 9- or 10-membered aromatic ring system; or (iii) a tricyclic 13- or 14-membered aromatic ring system. The aromatic 6- to 14-membered carbocyclic rings include, e.g., benzene, naphthalene, anthracene, indane, tetralin, and fluorene. As used herein, aryl refers to residues in which one or more rings are aromatic, but not all need be.

Heteroaryl means (i) a monocyclic 5- or 6-membered heteroaromatic ring containing 1-4 heteroatoms selected from O, N, or S; (ii) a bicyclic 9- or 10-membered heteroaromatic ring system containing 0-4 heteroatoms selected from O, N, or S; or (iii) a tricyclic 13- or 14-membered heteroaromatic ring system containing 0-5 heteroatoms selected from O, N, or S. The 5- to 10-membered aromatic heterocyclic rings include, e.g., thiophene, pyridine, pyrimidine, furan, imidazole, indole, benzopyranone, thiazole, benzimidazole, quinoline, isoquinoline, quinoxaline, pyrazine, tetrazole and pyrazole. As used herein, heteroaryl refers to residues in which one or more rings are aromatic, but not all need be.

Alkoxy (or alkoxyl) refers to groups of from 1 to 20 carbon atoms, or 1 to 10 carbon atoms, or 1 to 8 carbon atoms, or 1 to 6 carbon atoms, of straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Alkthio refers to groups of from 1 to 20 carbon atoms, or 1 to 10 carbon atoms, or 1 to 8 carbon atoms, or 1 to 6 carbon atoms, of straight or branched configuration attached to the parent structure through a sulfur.

The term “halogen” means fluorine, chlorine, bromine or iodine atoms.

The terms “haloalkyl,” “haloalkoxy,” or “haloalkylthio” mean alkyl, alkoxy, or alkylthio, respectively, substituted with one or more halogen atoms.

As used herein, the term “optionally substituted” may be used interchangeably with “unsubstituted or substituted.” The term “substituted” refers to the replacement of one or more hydrogen atoms in a specified group with a specified radical. For example, substituted alkyl, aryl, cycloalkyl, heterocyclyl, etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH₂], oxo [═O]alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine.

Exemplary compounds of the invention are shown below in Table 1. The examples do not limit the present invention.

TABLE 1

| Ex. # | Structure |
|---|---|
| SN 05 | 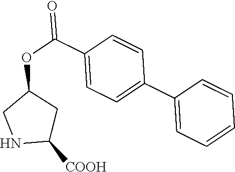 [SMILES: O=C(O[C@@H]1CN[C@H](C(=O)O)C1)c1ccc(-c2ccccc2)cc1] |
| SN 06 | 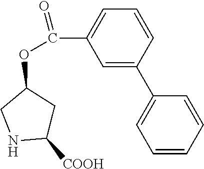 [SMILES: O=C(O[C@@H]1CN[C@H](C(=O)O)C1)c1cccc(-c2ccccc2)c1] |
| SN 07 | 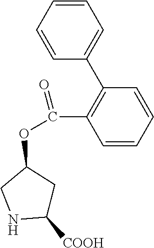 [SMILES: O=C(O[C@@H]1CN[C@H](C(=O)O)C1)c1ccccc1-c1ccccc1] |
| SN 08 | 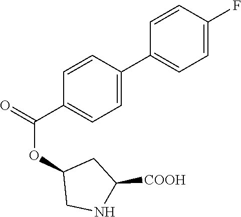 [SMILES: O=C(O[C@@H]1CN[C@H](C(=O)O)C1)c1ccc(-c2ccc(F)cc2)cc1] |
| SN 09 | 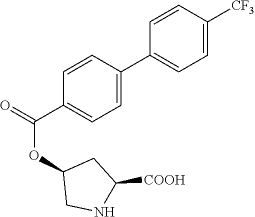 [SMILES: O=C(O[C@@H]1CN[C@H](C(=O)O)C1)c1ccc(-c2ccc(C(F)(F)F)cc2)cc1] |
| SN 10 | 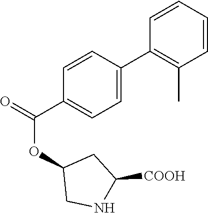 [SMILES: Cc1ccccc1-c1ccc(C(=O)O[C@@H]2CN[C@H](C(=O)O)C2)cc1] |
| SN 11 | 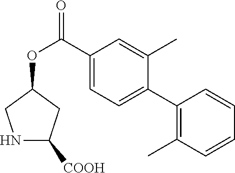 [SMILES: Cc1ccccc1-c1ccc(C(=O)O[C@@H]2CN[C@H](C(=O)O)C2)cc1C] |
| SN 12 | 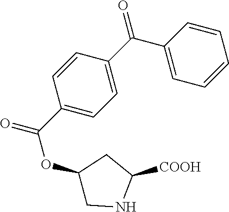 [SMILES: O=C(O[C@@H]1CN[C@H](C(=O)O)C1)c1ccc(C(=O)c2ccccc2)cc1] |
| SN 13 | 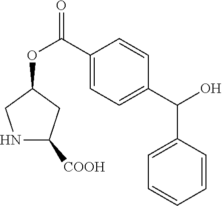 [SMILES: O=C(O[C@@H]1CN[C@H](C(=O)O)C1)c1ccc(C(O)c2ccccc2)cc1] |
| SN 14 | 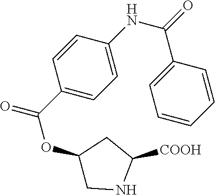 [SMILES: O=C(Nc1ccc(C(=O)O[C@@H]2CN[C@H](C(=O)O)C2)cc1)c1ccccc1] |
| SN 15 | 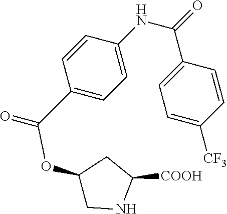 [SMILES: O=C(Nc1ccc(C(=O)O[C@@H]2CN[C@H](C(=O)O)C2)cc1)c1ccc(C(F)(F)F)cc1] |
| SN 16 | 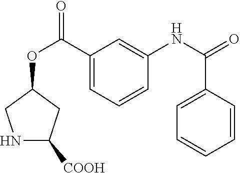 [SMILES: O=C(Nc1cccc(C(=O)O[C@@H]2CN[C@H](C(=O)O)C2)c1)c1ccccc1] |
| SN 17 | 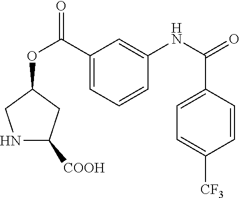 [SMILES: O=C(Nc1cccc(C(=O)O[C@@H]2CN[C@H](C(=O)O)C2)c1)c1ccc(C(F)(F)F)cc1] |
| SN 18 | 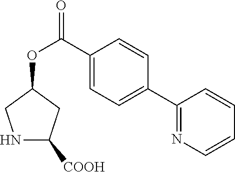 [SMILES: O=C(O[C@@H]1CN[C@H](C(=O)O)C1)c1ccc(-c2ccccn2)cc1] |
| SN 19 | 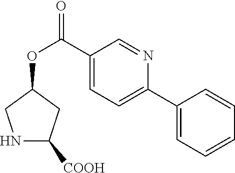 [SMILES: O=C(O[C@@H]1CN[C@H](C(=O)O)C1)c1ccc(-c2ccccc2)nc1] |
| SN 20 | 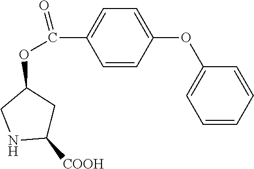 [SMILES: O=C(O[C@@H]1CN[C@H](C(=O)O)C1)c1ccc(Oc2ccccc2)cc1] |
| SN 21 | 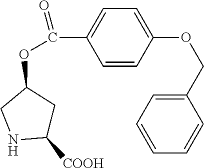 [SMILES: O=C(O[C@@H]1CN[C@H](C(=O)O)C1)c1ccc(OCc2ccccc2)cc1] |
| SN 22 | 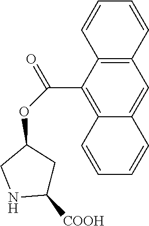 [SMILES: O=C(O[C@@H]1CN[C@H](C(=O)O)C1)c1c2ccccc2cc2ccccc12] |
| SN 23 | 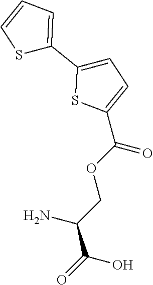 [SMILES: N[C@@H](COC(=O)c1ccc(-c2cccs2)s1)C(=O)O] |
| SN 24 | 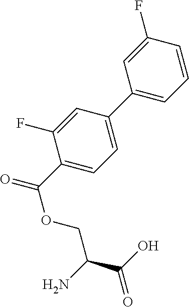 [SMILES: N[C@@H](COC(=O)c1ccc(-c2cccc(F)c2)cc1F)C(=O)O] |
| SN 25 | 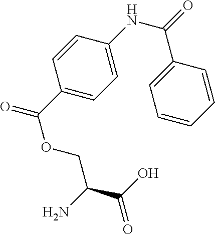 [SMILES: N[C@@H](COC(=O)c1ccc(NC(=O)c2ccccc2)cc1)C(=O)O] |
| SN 26 | 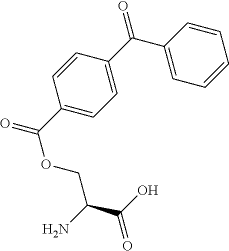 [SMILES: N[C@@H](COC(=O)c1ccc(C(=O)c2ccccc2)cc1)C(=O)O] |
| SN 27 | 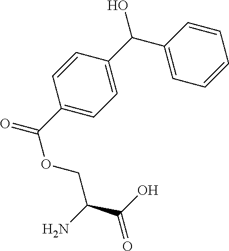 [SMILES: N[C@@H](COC(=O)c1ccc(C(O)c2ccccc2)cc1)C(=O)O] |
| SN 33 | 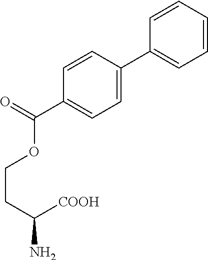 [SMILES: N[C@@H](CCOC(=O)c1ccc(-c2ccccc2)cc1)C(=O)O] |
| SN 34 | 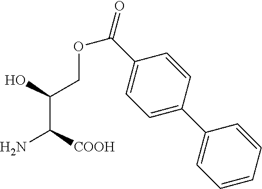 [SMILES: N[C@H](C(=O)O)[C@@H](O)COC(=O)c1ccc(-c2ccccc2)cc1] |
| SN 35 | 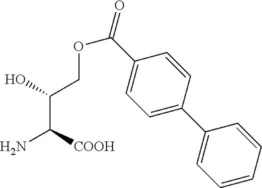 [SMILES: N[C@H](C(=O)O)[C@H](O)COC(=O)c1ccc(-c2ccccc2)cc1] |
| SN 36 | 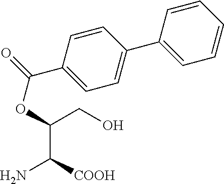 [SMILES: N[C@H](C(=O)O)[C@H](CO)OC(=O)c1ccc(-c2ccccc2)cc1] |
| SN 37 | 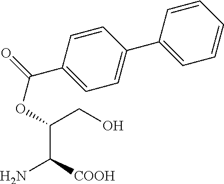 [SMILES: N[C@H](C(=O)O)[C@@H](CO)OC(=O)c1ccc(-c2ccccc2)cc1] |
| SN 38 | 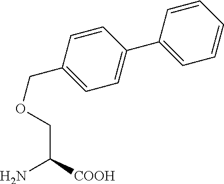 [SMILES: N[C@@H](COCc1ccc(-c2ccccc2)cc1)C(=O)O] |
| SN 39 | 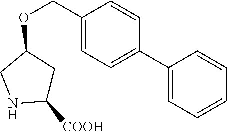 [SMILES: O=C(O)[C@@H]1C[C@H](OCc2ccc(-c3ccccc3)cc2)CN1] |
| SN 40 | 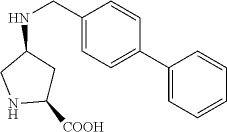 [SMILES: O=C(O)[C@@H]1C[C@H](NCc2ccc(-c3ccccc3)cc2)CN1] |
| SN 41 | 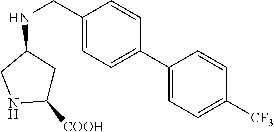 [SMILES: O=C(O)[C@@H]1C[C@H](NCc2ccc(-c3ccc(C(F)(F)F)cc3)cc2)CN1] |
| SN 42 | 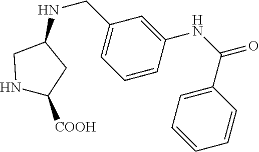 [SMILES: O=C(Nc1cccc(CN[C@@H]2CN[C@H](C(=O)O)C2)c1)c1ccccc1] |
| SN 43 | 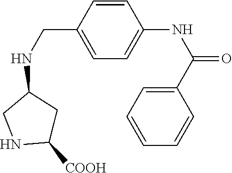 [SMILES: O=C(Nc1ccc(CN[C@@H]2CN[C@H](C(=O)O)C2)cc1)c1ccccc1] |
| SN 45 | 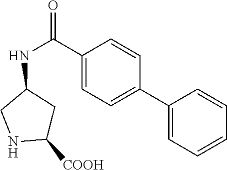 [SMILES: O=C(N[C@@H]1CN[C@H](C(=O)O)C1)c1ccc(-c2ccccc2)cc1] |
| SN 46 | 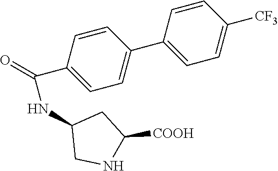 [SMILES: O=C(N[C@@H]1CN[C@H](C(=O)O)C1)c1ccc(-c2ccc(C(F)(F)F)cc2)cc1] |
| SN 47 | 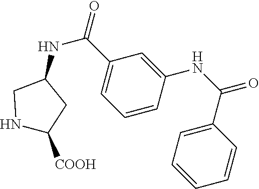 [SMILES: O=C(Nc1cccc(C(=O)N[C@@H]2CN[C@H](C(=O)O)C2)c1)c1ccccc1] |
| SN 48 | 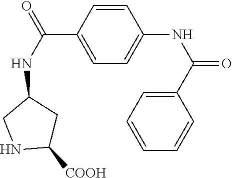 [SMILES: O=C(Nc1ccc(C(=O)N[C@@H]2CN[C@H](C(=O)O)C2)cc1)c1ccccc1] |
| SN 50 | 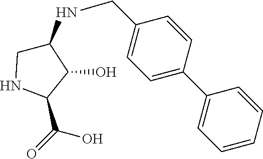 [SMILES: O=C(O)[C@H]1NC[C@@H](NCc2ccc(-c3ccccc3)cc2)[C@@H]1O] |
| SN 55 | 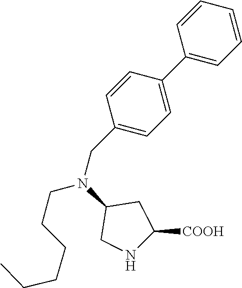 [SMILES: CCCCCCN(Cc1ccc(-c2ccccc2)cc1)[C@@H]1CN[C@H](C(=O)O)C1] |
| SN 56 | 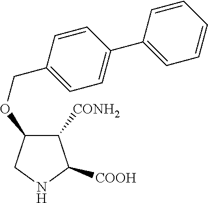 [SMILES: NC(=O)[C@@H]1[C@@H](C(=O)O)NC[C@H]1OCc1ccc(-c2ccccc2)cc1] |

In some embodiments, Example Number SN 05 is excluded from the claims.

### Experimental Section

In general, the production methods for the compounds of the present invention are explained with the following:

The starting materials and reagents used for each step in the following production methods, and the obtained compounds may each form a salt. Examples of the salts include those similar to the aforementioned salts of the compound of the present invention and the like.

When the compound obtained in each step is a free compound, it may be converted to a desired salt by methods well-known in the art. Conversely, when the compound obtained in each step is a salt, it may be converted to a free form or a desired other kind of salt by methods well-known in the art.

The compound obtained in each step may also be used for the next reaction as a reaction mixture thereof or after obtaining a crude product thereof. Alternatively, the compound obtained in each step may be isolated and/or purified from the reaction mixture by a separation means such as concentration, crystallization, recrystallization, distillation, solvent extraction, fractionation, chromatography and the like according to a conventional method.

When the starting materials and reagent compounds of each step are commercially available, the commercially available products are often used as is.

In each step, protection or deprotection of a functional group is performed by the method known per se, for example, the methods described in “Protective Groups in Organic Synthesis, 4th Ed.” (Theodora W. Greene, Peter G. M. Wuts) Wiley-Interscience, 2007; “Protecting Groups 3rd Ed.” (P. J. Kocienski) Thieme, 2004 and the like, or the methods described in the Examples.

The synthesis of exemplary compounds of the invention are shown below. The examples do not limit the present invention and the present invention can be modified within the scope of the present invention.

Synthesis

Chemicals were purchased from VWR or Sigma-Aldrich. Except for one isomer, all other compounds were synthesized following the same general procedure as shown in the reaction scheme below.

General Procedures

### Scheme 1 & 2: Synthesis of Serine, cis and trans-hydroxyproline derivatives

Scheme 1 step (i): (2S,4S)-(tert-butoxycarbonyl)-4-hydroxypyrrolidine-2-carboxylic acid (500 mg, 2.16 mmol) and imidazole (744 mg, 10.80 mmol) were weighed into an oven-dried round bottomed flask (RBF) and dissolved in dry DMF (6 mL). The reaction mixture was cooled to 0° C. and TBSCI in dry DMF (652 mg, 4.32 mmol) was added dropwise under N₂ gas. The reaction mixture was then left to warm up to room temperature and stirred for 24 hours.

After removal of excess DMF using N₂ gas at 50° C., the residue was suspended in ethyl acetate and washed twice with water, thrice with chilled 1 M HCl and once with brine. The organic layer was dried over anhydrous Na₂SO₄ and filtered. The filtrate was concentrated under reduced pressure to a colorless oil. The oil was dissolved in methanol (3 mL) and THE (4 mL) and the solution cooled to 0° C. LiOH H₂O (227 mg, 5.40 mmol) in water (3 mL) was added dropwise and the mixture was allowed to warm to room temperature and stirred for 2 hours. The pH of the solution was adjusted to 2-3 using chilled 1 M HCl and the product 2 (i) was collected as a pure, white precipitate after suction filtration.

Scheme 1 step (ii): (2S,4S)-1-(tert-butoxycarbonyl)-4-((tert-butyldimethylsilyl)oxy)pyrrolidine-2-carboxylic acid (661 mg, 1.91 mmol) was dissolved in dry DCM and cooled to 0° C. 1.03 mL of tert-Butyl 2,2,2-trichloroacetimidate (1,252 mg, 5.73 mmol) was added dropwise under N₂ and the mixture was stirred for 36 hours at room temperature. Excess solvent was removed in vacuo and the residue purified using flash silica gel chromatography (0-15% ethyl acetate in hexanes) to obtain 3 (i) as a pure colorless oil.

Scheme 1 step (iii): di-tert-butyl (2S,4S)-4-((tert-butyldimethylsilyl)oxy)pyrrolidine-1,2-dicarboxylate (635 mg, 1.58 mmol) was dissolved in dry THE and cooled to 0° C. 1M TBAF in THE (2.05 mL, 2.05 mmol) was added dropwise under N₂ atmosphere and reaction left to stir for 2 hours. Contents were diluted 25% ethyl acetate in hexanes and washed saturated NH₄Cl (1×), chilled 0.5M HCl (1×), NaHCO₃ (1×) and 1:1 mixture of H₂O and brine (1×). The organic layer was dried over Na₂SO₄ and filtered. The filtrate was concentrated in vacuo and the colorless oil formed was used in the next step without further purification. For 3 (i) L—cis isomer, the product was purified using flash silica gel chromatography (25-60% ethyl acetate in hexanes) to obtain a colorless oil which was precipitated from DCM with hexanes to obtain 4 (iii) as a pure white solid.

Scheme 1 step (iv): di-tert-butyl (2S,4S)-4-hydroxypyrrolidine-1,2-dicarboxylate (52 mg, 0.18 mmol), DMAP (4 mg, 0.04 mmol), biphenyl]-4-carboxylic acid (144 mg, 0.72 mmol) were dissolved in dry DCM and cooled to 0° C. DCC in dry DCM (75, 0.36 mmol) was added dropwise under N₂ gas and the reaction mixture was allowed to warm up to room temperature and was stirred for 48 hours. The contents were filtered off and the filtrate concentrated in vacuo. The residue was suspended in 50% ethyl acetate in hexanes and washed with NaHCO₃ (3×), chilled 0.5M HCl (3×), brine (3×) and H₂O (1×). The organic phase was collected and dried over Na₂SO₄ and filtered. The filtrate was concentrated in vacuo and the product purified using flash silica gel chromatography (10-50% ethyl acetate in hexanes) to obtain a pure 7 (a) as a white solid. The same procedure was followed in Scheme 2 step (vi).

Scheme 1 step (v): di-tert-butyl (2S,4S)-4-(([1,1′-biphenyl]-4-carbonyl)oxy)pyrrolidine-1,2-dicarboxylate (74 mg, 0.16 mmol) and DTT (49 mg, 0.32 mmol) were weighed into a 10 mL long-necked RBF and dissolved in dry DCM and cooled to 0° C. Trifluoroacetic acid (TFA) (0.39 mL, 5.12 mmol) was added dropwise under N₂ gas and the reaction mixture was stirred at room temperature for 36 hours. TFA was completely removed under reduced pressure and the product was collected as a white solid after trituration in methanol and diethyl ether. The product was confirmed by TLC (20-40% methanol in DCM), rf 0.2) and was used in all experiments without further purification. The same procedure was followed in Scheme 2 step (v).

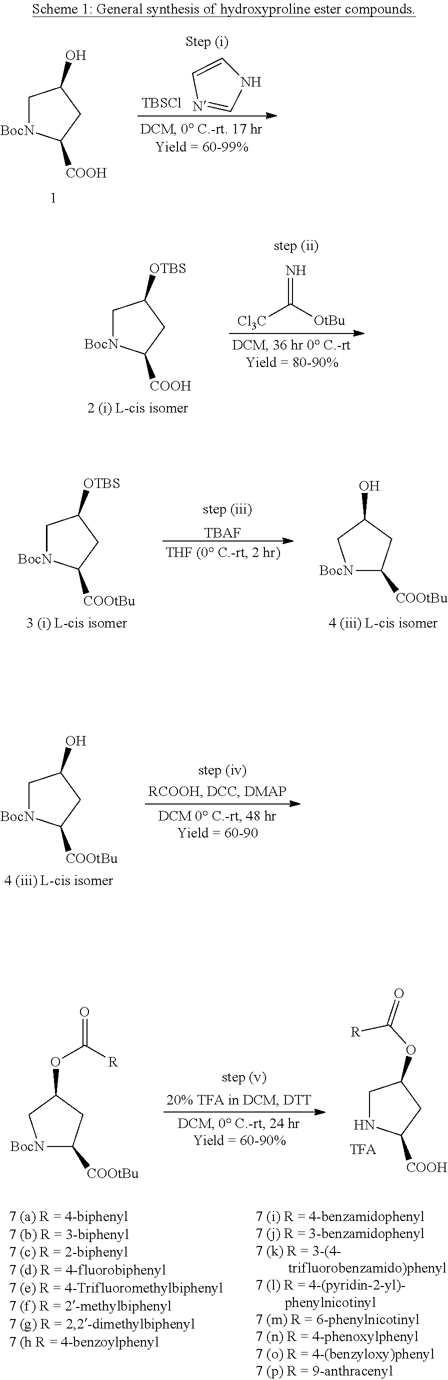

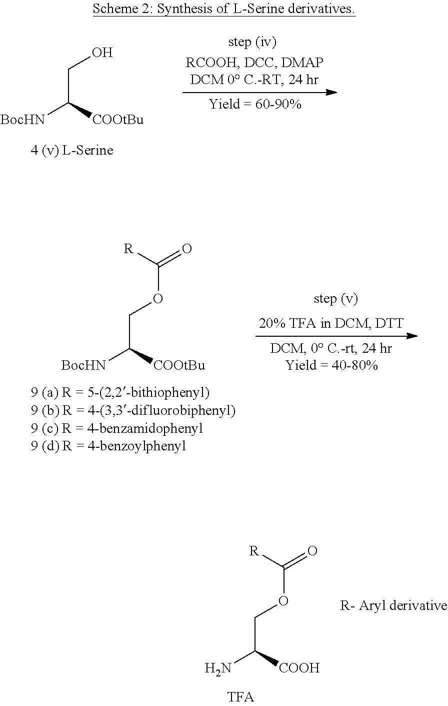

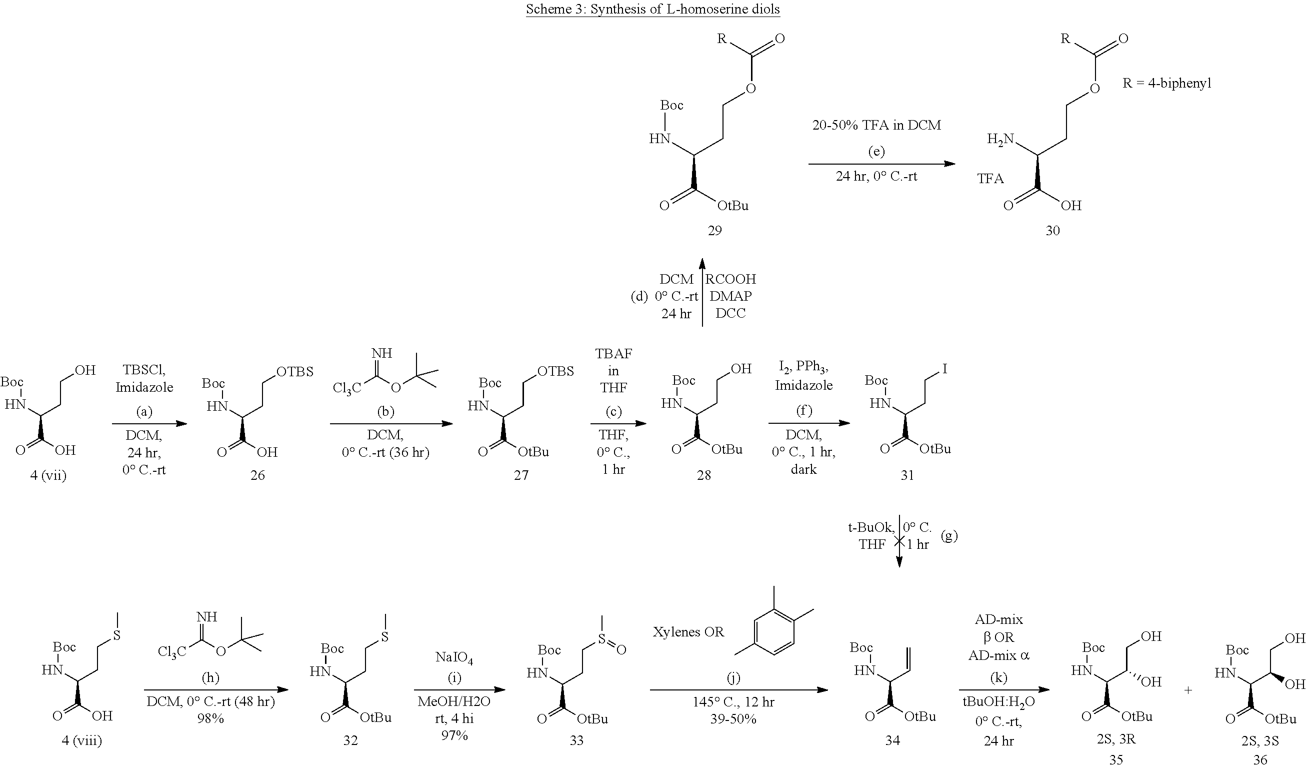

### Procedure for Synthesis of L-Homoserine Diols (Scheme 3)

Scheme 3: step (a) (tert-butoxycarbonyl)-L-homoserine, 4 (vii) (2,000 mg, 9.12 mmol) and imidazole (1,863 mg, 27.37 mmol) were dissolved in dry DCM (12 mL) and catalytic DMF (1 mL) then cooled to 0° C. TBSCI (1,787 mg, 11.86 mmol) in DCM was added dropwise under N₂ gas. The reaction mixture was then left to warm up to room temperature and stirred for 24 hours. 10 mL of chilled 1M HCl was added and reaction stirred for 30 minutes at 0° C. The aqueous layer was extracted with DCM (3×30 mL). The combined organic layers were dried over anhydrous Na₂SO₄, filtered off and the filtrate concentrated under reduced pressure to a colorless oil residue. The residue was dissolved in methanol (6 mL) and water (10 mL) and pH of the solution was adjusted to 2-3 using chilled 1 M HCl. The product, 26 was collected as a pure solid after suction filtration. Product details; 2, 890 mg, 95% yield as a white powder.

Scheme 3: Step (b)N-(tert-butoxycarbonyl)-O-(tert-butyldimethylsilyl)-L-homoserine, 26 (1,400 mg, 4.21 mmol) was dissolved in dry DCM and cooled to 0° C. 2.3 mL of tert-Butyl 2,2,2-trichloroacetimidate (2,757 mg, 12.62 mmol) was added dropwise under N₂ and the mixture was stirred for 36 hours at room temperature. Precipitates were filtered out and filtrate concentrated in vacuo. The residue was purified using flash silica gel chromatography (0-15% ethyl acetate in hexanes) to obtain a pure colorless oil. Product details; 1,476 mg, 90% yield as a colorless oil.

Scheme 3: Step (c) tert-butyl N-(tert-butoxycarbonyl)-O-(tert-butyldimethylsilyl)-L-homoserinate, 27 (1,680 mg, 4.31 mmol) was dissolved in dry THE (10 mL) and cooled to 0° C. 1M TBAF in THE (5.6 mL, 5.60 mmol) was added dropwise under N₂ atmosphere and reaction left to stir for 2 hours. Contents were diluted 10 mL ethyl acetate and washed with saturated NH₄Cl (2×15 mL), NaHCO₃ (2×15 mL), brine (1×15 mL) and water (1×15 mL). The organic layer was dried over Na₂SO₄ and filtered. The filtrate was concentrated in vacuo and the colorless oil. The product was purified using flash silica gel chromatography (25%-60% ethyl acetate in hexanes). Product details; 1,020 mg, 86% yield as a colorless oil.

Scheme 3: Step (d) tert-butyl (tert-butoxycarbonyl)-L-homoserinate, 28 (76 mg, 0.28 mmol), DMAP (6.7 mg, 0.06 mmol), biphenyl]-4-carboxylic acid (219 mg, 1.10 mmol) were dissolved in dry DCM (5 mL) and DMF (1 mL) then cooled to 0° C. DCC (68, 0.33 mmol) in DCM was added dropwise under N₂ gas and the reaction mixture left to warm up to room temperature and stirred for 24 hours. The contents were filtered off and the filtrate concentrated in vacuo. The residue was suspended in 50% ethyl acetate in hexanes and washed with NaHCO₃ (3×10 mL), chilled 0.5M HCl (3×10 mL), brine (3×10 mL) and H₂O (1×10 mL). The organic phase was collected and dried over Na₂SO₄ and filtered off. The filtrate was concentrated in vacuo and the product purified using flash silica gel chromatography (10-40% ethyl acetate in hexanes). Product details; 61 mg, 48% yield as a colorless oil.

Scheme 3: Step (e) (S)-4-(tert-butoxy)-3-((tert-butoxycarbonyl)amino)-4-oxobutyl [1,1′-biphenyl]-4-carboxylate, 29 (60 mg, 0.13 mmol) and DTT (41 mg, 0.26 mmol) were dissolved in dry DCM and cooled to 0° C. Trifluoroacetic acid (TFA) (0.32 mL, 4.22 mmol) was added dropwise under N₂ and reaction mixture stirred at room temperature for 36 hours. TFA was completely removed under reduced pressure and product 30 was collected as a white solid after trituration in methanol and diethyl and used in all experiments without any further purification.

Scheme 3: Step (f) tert-butyl (tert-butoxycarbonyl)-L-homoserinate, 28 (58 mg, 0.21 mmol), PPh₃ (72 mg, 0.27 mmol), imidazole (43 mg, 0.63 mmol) and I2 (80 mg, 0.32 mmol) were dissolved in DCM (2 mL) and cooled to 0° C. then stirred in the dark for 1 hour at 0° C. then 2 hours at room temperature. Reaction mixture was diluted with 10 mL DCM and transferred into a separating funnel. Organic layer was washed with saturated cold Na₂S₂O₃ (2×5 mL) and brine (2×5 mL). Organic layer was dried over Na₂SO₄ and filtered off. The filtrate was concentrated in vacuo and product purified using flash silica gel chromatography (5-15% acetone in DCM). Product details; 75 mg, 92% yield as a brown oil.

Scheme 3: Step (h) (tert-butoxycarbonyl)-L-methionine, 4 (viii) (500 mg, 2.01 mmol), was dissolved in dry DCM and cooled to 0° C. 1.1 mL of tert-Butyl 2,2,2-trichloroacetimidate (1.31 mg, 6.02 mmol) was added dropwise under N₂ and the mixture stirred for 36 hours at room temperature. Precipitates were filtered out and the filtrate concentrated in vacuo. The residue was purified using flash silica gel chromatography (0-15% ethyl acetate in hexanes) to obtain a pure colorless oil. Product details; 615 mg, 100% yield as a colorless oil.

Scheme 3: Step (i) tert-butyl (tert-butoxycarbonyl)-L-methioninate, 32 (270 mg, 0.88 mmol) was dissolved in methanol (3 mL) and cooled to 0° C. NaIO₄ (212 mg, 0.99 mmol) in 3.5 mL water was added at 0° C. Reaction was let to warm up to room temperature and stirred for 4 hours. TLC showed complete conversion. Reaction mixture was diluted with water (10 mL) and transferred into separating funnel. Product was extracted with EtOAc (3×7 mL). Combined organic layers were dried over Na₂SO₄ and filtered off. The filtrate was concentrated in vacuo and product purified using flash silica gel chromatography (5%-80% acetone in DCM). Product details; 632 mg, 97% yield as a white powder.

Scheme 3: Step (j) tert-butyl (2S)-2-((tert-butoxycarbonyl)amino)-4-(methylsulfinyl)butanoate, 33 (94 mg, 0.29 mmol) was dissolved in xylenes or 1,2,4-trimethylbenzene and the mixture heated in a pressure tight glass vessel (or refluxed at 150° C.) at 150° C. over an oil bath for 12 hours. Excess solvent was removed by passing a stream of N₂ or by high vacuum. Product was purified by flash silica gel chromatography 0%-10% EtOAc in hexanes to obtain 34 as a colorless oil, 39%-60% yield.

Scheme 3: Step (k) AD-mix α (1,219 mg) was dissolved in t-BuOH (3 mL) and water (5 mL) at room temperature until all contents dissolved. The solution was then cooled to 0° C. and 34, tert-butyl (S)-2-((tert-butoxycarbonyl)amino)but-3-enoate, (224 mg, 0.87 mmol) in t-BuOH (5 mL) added. The mixture was stirred at 0° C. for 24-36 hours until complete conversion. The reaction was quenched by the addition of Na₂SO₃ (1,300 mg, 10.32 mmol) at 0° C. The mixture was let to stir at room temperature for 1 hour then transferred into separating funnel and diluted with DCM (15 mL) and brine (5 mL). Aqueous layer was extracted with DCM (3×10 mL). Combined DCM layers were washed with brine (3×15 mL) then dried over Na₂SO₄ and filtered off. The filtrate was concentrated in vacuo and product purified using flash silica gel chromatography (0%-90% EtOAc in hexanes). Products details; Top spot (2S, 3R) 35, 100 mg, Bottom spot (2S, 3S) 36, 80 mg and mixture (35, 36) 30 mg with combined yield of 83% as a colorless oil that turns solid on freezing. AD-mix-β follows same protocol and generates same products.

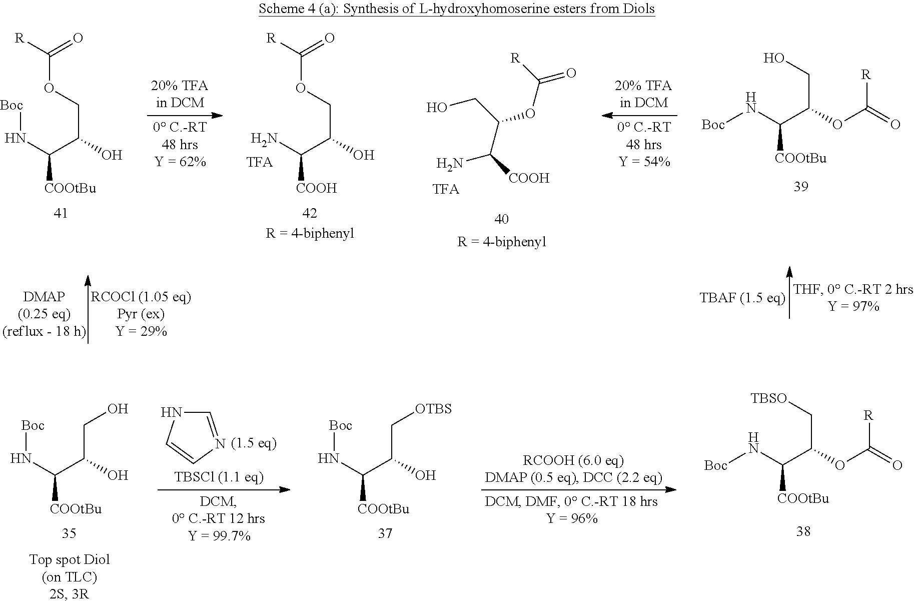

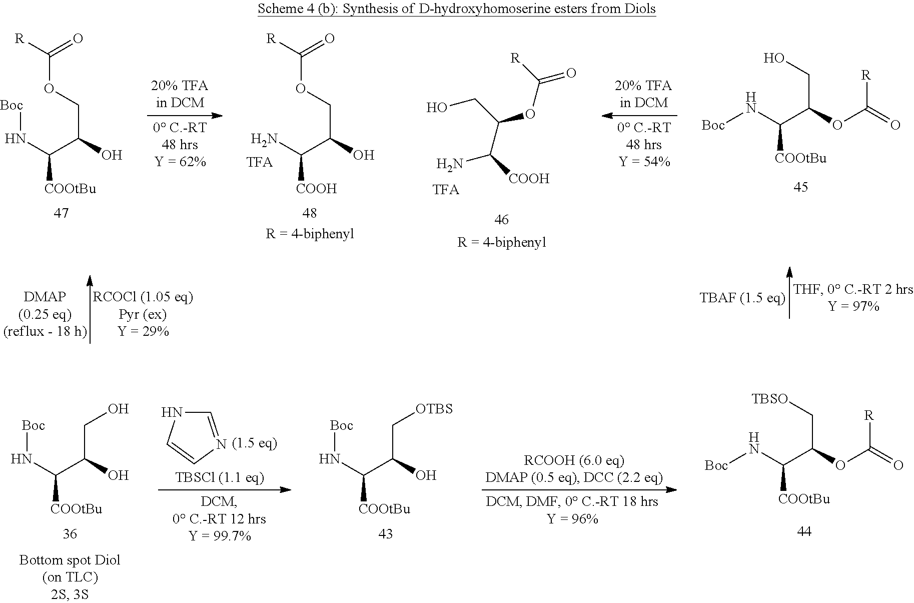

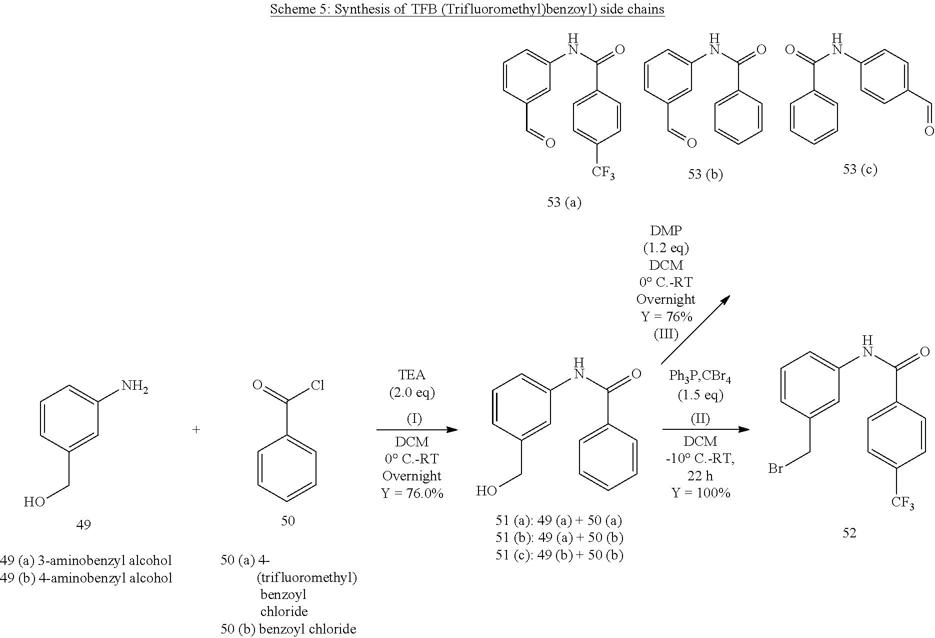

### General Procedure for Scheme 5: Synthesis of TFB (Trifluoromethyl)Benzoyl) Side Chains

Scheme 5: step (I): 3-aminobenzyl alcohol, 49 (a) (2.07 g, 16.78 mmol) was dissolved in DCM (20 mL). TEA (4.7 mL, 3.40 mmol) was added and solution cooled to 0° C. 4-(trifluoromethyl)benzoyl chloride, 50 (a) was added in three portions (each after 4 hours) and reaction stirred overnight at room temperature. TLC showed complete conversion. Reaction mixture was transferred into separating funnel and washed with 2:1 mixture of water and brine (2×30 mL) and NaHCO₃ (2×25 mL), chilled 1M HCl (1×25 mL) and water (1×25 mL). Organic layer was dried over Na₂SO₄ and filtered off. The filtrate was concentrated in vacuo and product purified using flash silica gel chromatography (0%-5% MeOH in DCM). Products details; 51 (a), 3.76 g, 76% yield as a white solid.

Scheme 5: step (II): 51 (a) (50 mg, 0.17 mmol), PPh₃ (67 mg, 0.25 mmol), CBr₄ (84 mg, 0.25 mmol) were dissolved in dry DCM and cooled to ˜10° C. The reaction was left to warm up to room temperature and stirred for 22 hours. The volatiles were removed under reduced pressure and residue purified using flash silica gel chromatography (5%-20% EtOAc in hexanes).

Products details; 52, 60 mg, 99% yield as a spongy white solid.

Scheme 5: step (III): A solution of 51 (a) (500 mg, 1.69 mmol) in DCM was cooled to 0° C. 8-12% DMP (862 mg, 2.03 mmol) was added dropwise and reaction left to stir at room temperature overnight. Reaction mixture was filtered out and DCM evaporated under reduced pressure. The residue was taken up in EtOAc and washed with Na₂S₂O₃ (3×15 mL) and mixture of NaHCO₃ and brine 5:1 (3×15 mL). Organic layer was dried over Na₂SO₄, filtered off and filtrate concentrated in vacuo to yield 53 (a), 375 mg, 76% yield as an orange-brown solid.

Synthesis of 51 (b): Same procedure as Scheme 5: step (I); 49 (a) (1.5 g, 12.18 mmol), TEA (3.4 mL, 24.36 mmol), benzoyl chloride, 50 (b) (1.88 g, 13.40 mmol). Products details; 51 (b), 2.71 g, 98% yield as a pale-yellow powder.

Synthesis of 51 (c): Same procedure as Scheme 5: step (I); 49 (b) (1.5 g, 12.18 mmol), TEA (3.4 mL, 24.36 mmol), benzoyl chloride, 50 (b) (1.88 g, 13.40 mmol). Products details; 51 (c), 1.66 g, 56% yield as a yellow solid.

Synthesis of 53 (b): Same procedure as Scheme 5: step (III); 51 (b) (1.00 g, 4.40 mmol), 8-12% DMP (2.24 g, 5.28 mmol) in 12 mL DCM. Product details; 0.99 g, 100% yield as solid used in the next step without further purification.

Synthesis of 53 (c): Same procedure as Scheme 5: step (III); 51 (c) (1.00 g, 4.40 mmol), 8-12% DMP (2.24 g, 5.28 mmol) in 12 mL DCM. Product details; 1.00 g, 100% yield as solid used in the next step without further purification.

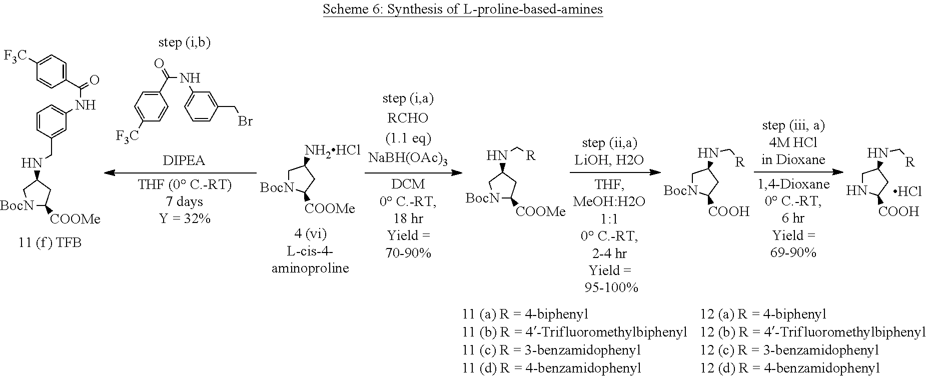

### General Procedure for the Synthesis of Amine; General Scheme 6

Scheme 6: step (i, a): 4 (vi), (200 mg, 0.71 mmol) and biphenyl-4-carbaldehyde (196 mg, 0.78 mmol, 1.1 eq) were dissolved in 6 mL of dry DCM in a 25 mL flask and cooled to 0° C. NaBH(OAc)₄ (313 mg, 1.42 mmol, 2.0 eq) in a single portion and reaction was let to stir at room temperature for 24 hours. TLC showed complete conversion. Reaction was quenched with 1 mL of water and stirred for an additional 1 hour. Contents were transferred into separating funnel and 10 mL water added. Aqueous layer was extracted with DCM 3×10 mL. Organic layer was dried over Na₂SO₄, filtered off and filtrate concentrated in vacuo. Residue was purified using flash silica gel column chromatography 25%-60% EtOAC in hexanes with eluent containing 3% TEA to yield colorless oil that turns to white solid on cooling.

Scheme 6: step (i, b): 4 (vi), (100 mg, 0.36 mmol, 1 eq) and corresponding benzyl bromide (127 mg, 0.36 mmol) were dissolved in THE and cooled to 0° C. DIPEA was added dropwise at 0° C. under N₂ and reaction stirred at room temperature and reaction stirred for 7 days. The reaction mixture was concentrated in vacuo and residue purified using flash silica gel column chromatography using 50%-100% EtOAc in hexanes to yield 59 mg, 32% yield, colorless oil.

Scheme 6: step (i, c): Similar to Scheme 6 step (i, a) procedure. Briefly, 4 (vi), (80 mg, 0.29 mmol) and respective aldehyde (166 mg, 0.71 mmol, 2.5 eq) were dissolved in 4 mL of dry DCM in a 10 mL flask and cooled to 0° C. NaBH(OAc)₄ (219 mg, 1.00 mmol, 3.5 eq) was added in three portions and reaction was let to stir at room temperature for 24 hours. TLC showed complete conversion. Reaction was quenched with 1 mL of water and stirred for an additional 1 hour. Contents were transferred into separating funnel and 10 mL water added. Aqueous layer was extracted with DCM (10 mL×3). Organic layer was dried over Na₂SO₄, filtered off and filtrate concentrated in vacuo. Residue was purified using flash silica gel column chromatography 10%-60% EtOAC in hexanes with eluent containing 1-3% TEA.

Scheme 6: step (i, d): Single coupled amine 11 (b), (60 mg, 0.13 mmol) and respective aldehyde (43 mg, 0.19 mmol, 2.0 eq) were dissolved in 4 mL of dry DCM in a 10 mL flask and cooled to 0° C. NaBH(OAc)₄ (55 mg, 0.25 mmol, 2.0 eq) in one portion and reaction was let to stir at room temperature for 24 hours. TLC showed complete conversion. Reaction was quenched with 1 mL of water and stirred for an additional 1 hour. Contents were transferred into separating funnel and 10 mL water added. Aqueous layer was extracted with DCM (10 mL×3). Organic layer was dried over Na₂SO₄, filtered off and filtrate concentrated in vacuo. Residue was purified using flash silica gel column chromatography 8%-40% EtOAC in hexanes with eluent containing 2.5% TEA.

Scheme 6: step (ii, a): Respective amine (100 mg, 0.21 mmol) was dissolved in THF and methanol and cooled to 0° C. LiOH·H₂O (61 mg, 1.46 mmol, 6.0 eq) in 1 mL water and reaction was let to warm up to room temperature and stirred for 4 hours. Excess THF was removed in vacuo and contents transferred into separating funnel using 10 mL ether. 5 mL water was then added and pH of aqueous phase adjusted to 8.0 (all precipitates dissolves) and extracted with ether and hexane (1:1) (10 mL×3). 20 mL of DCM was added and pH of aqueous later readjusted to 3.0-4.0 and extracted with DCM (20 mL×3) and EtOAc (20 mL×3). The combined organic layers were dried over Na₂SO₄, filtered off and filtrated concentrated in vacuo and residue further dried under line vacuum to yield analytically pure white solids.

Scheme 6: step (ii, b) and step (ii, c): Respective amine (100 mg, 0.21 mmol) was dissolved in THF and methanol and cooled to 0° C. LiOH·H₂O (61 mg, 1.46 mmol, 6.0 eq) in 1 mL water and reaction was let to warm up to room temperature and stirred for 4 hours. Excess THF was removed in vacuo and contents transferred into separating funnel using 10 mL ether. 5 mL water was then added and pH of aqueous phase adjusted to ˜ 3.0-4.0 using chilled 3M HCl and extracted with 50% EtOAc in hexanes (15 mL×3). The combined organic layers were dried over Na₂SO₄, filtered off and filtrated concentrated in vacuo and the residue purified using flash silica gel column chromatography 5%-8% MeOH in DCM with eluent containing 1% TEA.

Scheme 6: step (iii, a), step (iii, b) and step (iii, c): Amine bearing Boc group (₄₀ mg, 0.09 mmol) was dissolved in 1.5 mL of dry 1,4-dioxane and cooled to 0° C. 4M HCl (1.5 mL, 5.85 mmol, 65.0 eq) in 1,4 dioxane was added dropwise under N₂. The reaction was left to warm up to room temperature and stirred overnight. TLC showed complete conversion. Excess Dioxane was removed in vacuo by repeatedly adding DCM (3 mL×3) and evaporating under reduced pressure until a white powder formed. The powder was suspended in 3 mL of EtOAc and sonicated for 10 minutes and filtered off to yield analytically pure solids as confirmed by TLC (20%-40% MeOH in DCM).

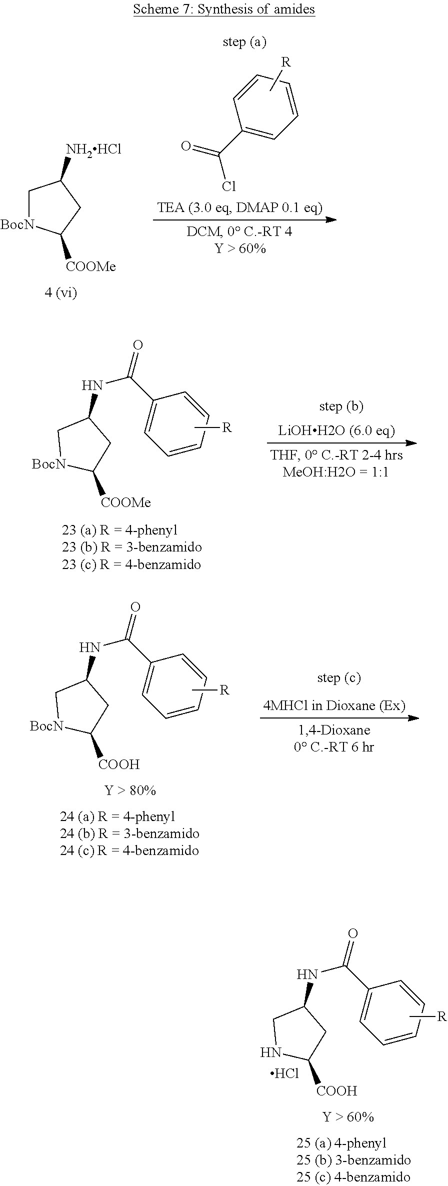

### General Protocol for Scheme 7: Synthesis of Amides

Scheme 7: step (a): 4 (vi) (100 mg, 0.36 mmol), TEA (0.15 mL, 1.07 mmol, 3.0 eq) and DMAP (4.4 mg, 0.04 mmol, 0.1 eq) were dissolved in DCM and solution cooled to 0° C. Biphenyl-4-carbonyl chloride (77 mg, 0.37 mmol, 1.05 eq) in DCM was added slowly and reaction was let to warm up to room temperature and stirred for 3 hours. Reaction was quenched with 1 mL water and stirred for 30 minutes. Excess DCM was removed in vacuo and residue taken up in 15 mL of 50% EtOAc in hexanes and transferred into separating funnel. Organic layer was washed with chilled 1M HCl (3×10 mL), NaHCO₃ (3×10 mL) and water (1×10 mL). The organic layer was dried over Na₂SO₄, filtered off and filtrated concentrated in vacuo and the residue purified using flash silica gel column chromatography 15%-40% EtOAc in hexanes.

Scheme 7: step (b): Respective amide 23 (90 mg, 0.21 mmol) was dissolved in THE and methanol and cooled to 0° C. LiOH·H₂O (53 mg, 1.27 mmol, 6.0 eq) in 1 mL water and reaction was let to warm up to room temperature and stirred for 4 hours. Excess THF was removed in vacuo and contents transferred into separating funnel. 7 mL water, 10 mL ether and 4 mL hexanes were added. Aqueous layer, at pH ˜12-14 was washed with 5:2 ether and hexanes mixture (3×14 mL) and organic layers discarded. 10 mL DCM was then added and pH of aqueous phase adjusted to ˜ 2.0-3.0 using chilled 3M HCl then extracted with DCM (10 mL×3). The combined organic layers were dried over Na₂SO₄, filtered off and filtrated concentrated in vacuo to yield analytically pure white solid used in the next without further purification.

Scheme 7: step (c): Boc protected amide 24 (40 mg, 0.10 mmol) was dissolved in 2.0 mL of dry 1,4-dioxane and cooled to 0° C. 4M HCl (1.6 mL, 6.33 mmol, 65.0 eq) in 1,4 dioxane was added dropwise under N₂. The reaction was left to warm up to room temperature and stirred for 3 hours then sonicated for an additional 2 hours until TLC showed complete conversion. Excess Dioxane was removed in vacuo by repeatedly adding DCM (2×3 mL) and evaporating under reduced pressure until a white powder formed. The powder was suspended in 1 mL of DCM and 3 mL of EtOAc and sonicated for 30 minutes and filtered off to yield analytically pure solid.

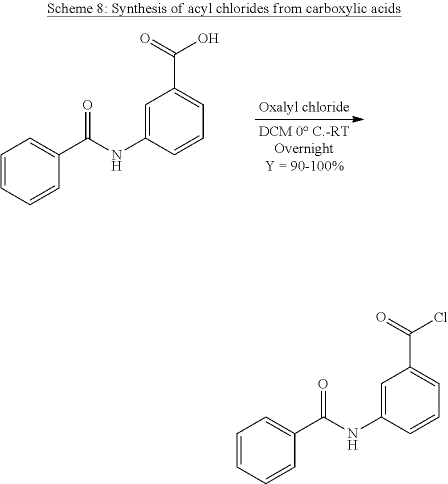 [SMILES: O=C(O)c1cccc(NC(=O)c2ccccc2)c1 ; O=C(Cl)c1cccc(NC(=O)c2ccccc2)c1]

### General Protocol for Synthesis of Acyl Chlorides from Carboxylic Acids; Scheme 8

Carboxylic acid (1.0 eq) was dissolved in DCM under N₂ gas and flask cooled to 0° C. DMF (catalytic, 0.1 mL) was added followed by slow addition of oxalyl chloride (1.1 eq). The reaction was let to stir overnight at room temperature. Excess solvent was removed under reduced pressure by addition of DCM (4×3 mL) and product was further dried in line vacuum. Dried acyl chlorides were used in the next step without any further purifications.

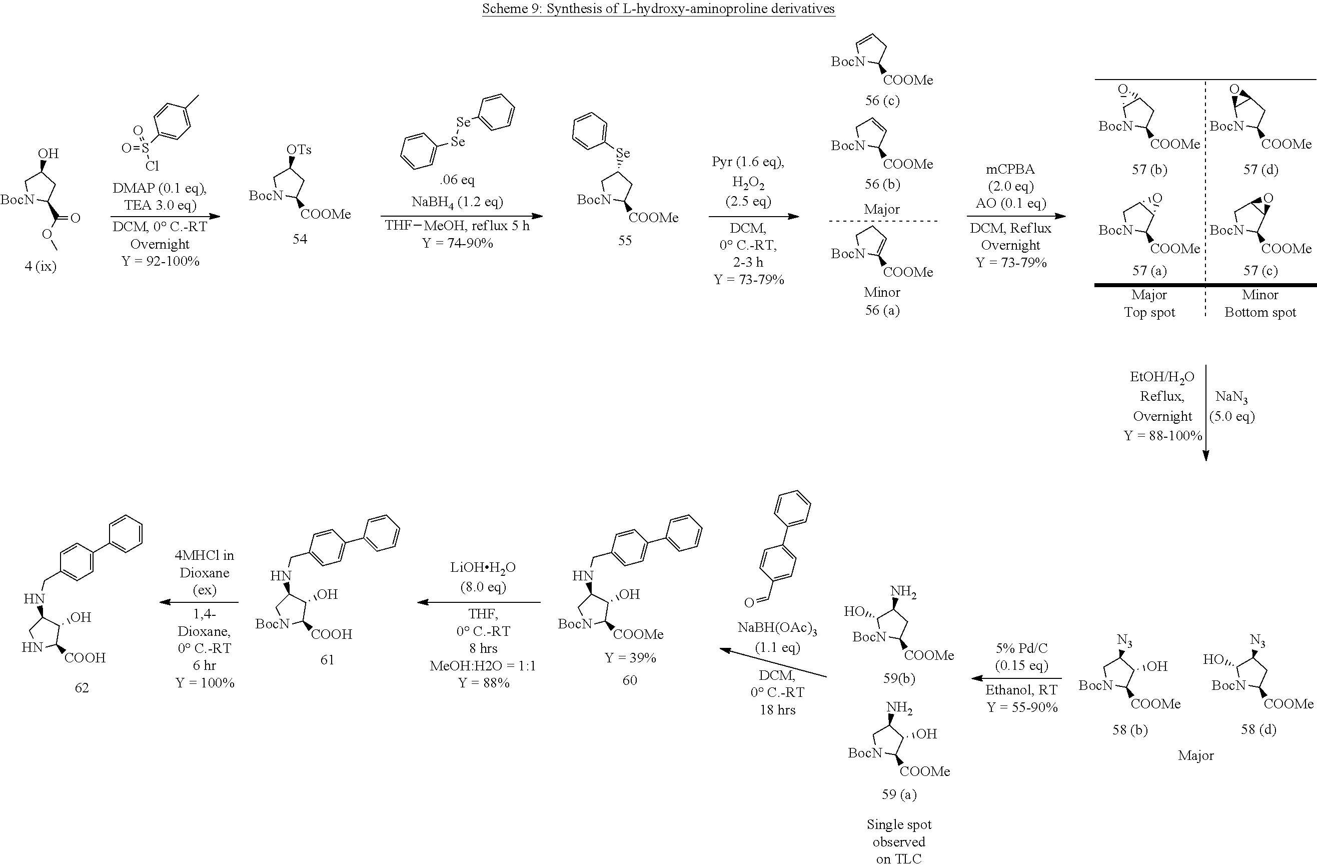

### Procedures for Scheme 9 Intermediates

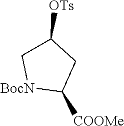

1-(tert-butyl) 2-methyl (2S,4S)-4-(tosyloxy)pyrrolidine-1,2-dicarboxylate, 54. Scheme 9 step (i); 4 (ix) (1000 mg, 3.48 mmol), TEA (1.5 mL, 10.44 mmol) and DMAP (43 mg, 0.35 mmol) were dissolved in DCM (10.0 mL) and solution cooled to 0° C. Toluene sulfonyl chloride (855 mg, 4.49 mmol) in DCM (3.0 mL) was added slowly and reaction was let to warm up to room temperature and stirred overnight. Excess DCM was removed in vacuo and residue taken up in 20 mL of 50% EtOAc in hexanes and transferred into separating funnel. Organic layer was washed with NaHCO₃ (2×10 mL), chilled 1M HCl (2×10 mL), and 4:1 mixture of brine and water (2×10 mL). The organic layer was dried over Na₂SO₄, filtered off and filtrated concentrated in vacuo and the residue purified using flash silica gel column chromatography 15%-40% EtOAc in hexanes. Product; 1.421 g, 92% as a colorless oil.

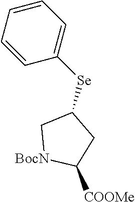 [SMILES: COC(=O)[C@@H]1C[C@@H]([Se]c2ccccc2)CN1C(=O)OC(C)(C)C]

1-(tert-butyl) 2-methyl (2S,4R)-4-(phenylselanyl)pyrrolidine-1,2-dicarboxylate, 55. Scheme 9 step (ii); Diphenyl diselenide (0.803 g, 2.57 mmol) and NaBH4 (0.131 g, 3.49 mmol) were charged into a three-necked-flask and 1:1 mixture of THF and MeOH added (16 mL) added under N₂. The solution was then refluxed at 80° C. for 20 minutes. 54 (1.714 g, 4.29 mmol) in THE (7 mL) was added dropwise at room temperature Reaction was brought to reflux again for 7 hours. 0.500 g of NaBH4 was added in two portions and reaction stirred for an additional 12 hours. Reaction was quenched with water and contents diluted with 50 mL 1:1 mixture of EtOAc and hexanes and transferred into separating funnel. Aqueous layer was washed with water (3×50 mL) and the organic layer dried over Na₂SO₄, filtered off and filtrated concentrated in vacuo and the residue purified using flash silica gel column chromatography 0%-25% EtOAc in hexanes. Product; 1.483 g, 90% as a colorless oil.

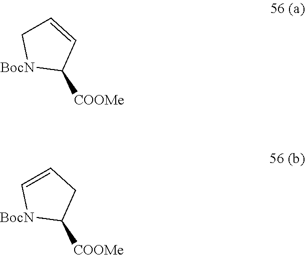 [SMILES: COC(=O)[C@@H]1C=CCN1C(=O)OC(C)(C)C ; COC(=O)[C@@H]1CC=CN1C(=O)OC(C)(C)C]

1-(tert-butyl) 2-methyl (S)-2,5-dihydro-1H-pyrrole-1,2-dicarboxylate, 56. Scheme 9 step (iii); 55 (750 mg, 1.95 mmol) and pyridine (0.25 mL, 3.12 mmol) were dissolved in DCM and solution cooled to 0° C. 30% H₂O₂ was added dropwise and reaction stirred for 2 hours at room temperature until 100% conversion on TLC. Reaction mixture was transferred into separating funnel and washed with chilled 1M HCl (3×10 mL), saturated Na₂SO₃ (3×10 mL) and 1:1 mixture of brine and water (2×10 mL). Organic layer dried over Na₂SO₄, filtered off and filtrated concentrated in vacuo and the residue purified using flash silica gel column chromatography 0%-40% EtOAc in hexanes to give three major possible isomers, 56 (a), 56 (b) and 56 (c). 56 (c) was separated out. Product, 56 (a) and 56 (b); 350 g, 79% as a clear pale-yellow oil.

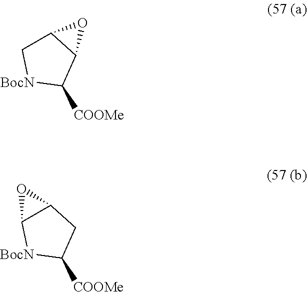 [SMILES: COC(=O)[C@@H]1[C@H]2O[C@H]2CN1C(=O)OC(C)(C)C ; COC(=O)[C@@H]1C[C@H]2O[C@H]2N1C(=O)OC(C)(C)C]

3-(tert-butyl) 2-methyl (1R,2S,5S)-6-oxa-3-azabicyclo[3.1.0]hexane-2,3-dicarboxylate, 57 (a). Scheme 9 step (iv); 56 (232 mg, 1.02 mmol), anti-oxidant (4,4′-Thiobis(6-tert-butyl-m-cresol), AO (37 mg, 0.10 mmol) and mCPBA (167 mg, 0.97 mmol) were dissolved in 8 mL DCM under N₂ then refluxed (50° C.) for 16 hours. Additional mCPBA (229 mg) was added and refluxed for an additional 15 hours. Reaction mixture was then cooled to room temperature then to 0° C. The white precipitate was filtered off and washed with chilled DCM. The filtrate was evaporated under reduced pressure. The residue was taken up in ether and transferred into separating funnel, washed with NaHCO₃ (5×10 mL) and 1:1 brine water mixture (2×10 mL). Ether layer was dried over Na₂SO₄, filtered off and filtrated concentrated in vacuo and the residue purified using flash silica gel column chromatography 25%-80% EtOAc in hexanes to obtain major trans isomers 57 (a) and 57 (b), 55% indicated as ‘top spot’ on TLC and minor cis isomers 57 (c) and 57 (d), 24%; all clear pale-yellow oils.

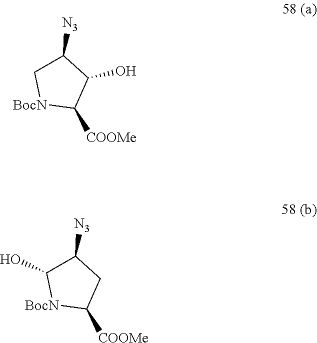 [SMILES: COC(=O)[C@@H]1[C@@H](O)[C@H](N=[N+]=[N-])CN1C(=O)OC(C)(C)C ; COC(=O)[C@@H]1C[C@H](N=[N+]=[N-])[C@@H](O)N1C(=O)OC(C)(C)C]

1-(tert-butyl) 2-methyl (2S,3R,4R)-4-azido-3-hydroxypyrrolidine-1,2-dicarboxylate, 58 (a). Scheme 9 step (v); 57 (100 mg, 0.41 mmol), NH₄Cl (44 mg, 0.82 mmol) and NaN3 (160 mg, 2.47 mmol) were dissolved in 6 mL ethanol then water (2 mL) was added. The mixture was refluxed at 70° C. overnight. Excess solvent was removed in vacuo and residue take up in EtOAc and transferred into separating funnel then washed with water (3×15 mL). Organic layer was dried over Na₂SO₄, filtered off and filtrated concentrated in vacuo and the residue (single spot on TLC) used in the next step without further purification, Product; (mixture of possible products with 58 (a) and 58 (b) as major isomers, 103 mg, 88%; as colorless oil.

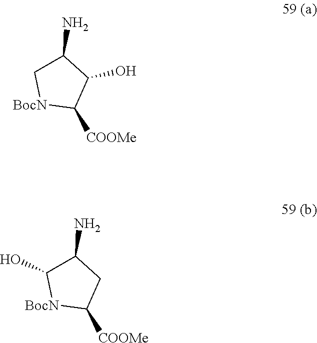 [SMILES: COC(=O)[C@@H]1[C@@H](O)[C@H](N)CN1C(=O)OC(C)(C)C ; COC(=O)[C@@H]1C[C@H](N)[C@@H](O)N1C(=O)OC(C)(C)C]

1-(tert-butyl) 2-methyl (2S,3S,4R)-4-amino-3-hydroxypyrrolidine-1,2-dicarboxylate, 59 (a). Scheme 9 step (vi); 58 (100 mg, 0.41 mmol) and 5% Pd/C (22 mg, 0.01 mmol) were dissolved in 6 mL ethanol under N₂. Hydrogen gas in a balloon was bubbled through the mixture for 2 hours at room temperature. Mixture was filtered through celite and filtrate evaporated under reduced pressure. The residue was passed through silica plug using 50%-100% EtOAc in hexanes with 2% TEA. The residue was used in the next step without further purification; 84 mg, 90% as a colorless oil.

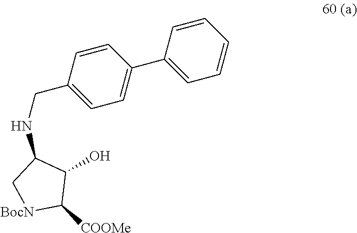 [SMILES: COC(=O)[C@@H]1[C@@H](O)[C@H](NCc2ccc(-c3ccccc3)cc2)CN1C(=O)OC(C)(C)C]

1-(tert-butyl) 2-methyl (2S,3S,4R)-4-(([1,1′-biphenyl]-4-ylmethyl)amino)-3-hydroxypyrrolidine-1,2-dicarboxylate, 60 (a). Scheme 9 step (vii); 59 (a) (84 mg, 0.32 mmol) and biphenyl-4-cabaldehyde (71 mg, 0.39 mmol) were dissolved in 6 mL of dry DCM and cooled to 0° C. NaBH(OAc)₃ (178 mg, 0.81 mmol) added in two portions and reaction was let to stir at room temperature for 24 hours. TLC showed complete conversion. Reaction was quenched with acetone and concentrated in vacuo. Residue was taken up in ETOAc and transferred into separating funnel and washed with water (3×10 mL). Organic layer was dried over Na₂SO₄, filtered off and filtrate concentrated in vacuo. Residue was purified using flash silica gel column chromatography 10%-100% EtOAC in hexanes to yield 60 (a), 54 mg, 39% and 60 (b), 70 mg, 37% both as clear oils.

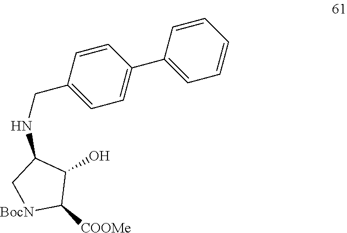 [SMILES: COC(=O)[C@@H]1[C@@H](O)[C@H](NCc2ccc(-c3ccccc3)cc2)CN1C(=O)OC(C)(C)C]

(2S,3S,4R)-4-(([1,1′-biphenyl]-4-ylmethyl)amino)-1-(tert-butoxycarbonyl)-3-hydroxypyrrolidine-2-carboxylic acid, 61. Scheme 9 step (viii, a); 60 (a) (54 mg, 0.13 mmol) was dissolved in THE (3 mL) and methanol (2 mL) and cooled to 0° C. LiOH·H₂O (43 mg, 1.01 mmol) in 2 mL water. Reaction was let to warm up to room temperature and stirred overnight. Excess THF was removed in vacuo and contents transferred into separating funnel using 10 mL ether. 5 mL water was then added and pH of aqueous phase adjusted to ˜ 3.0-4.0 using chilled 3M HCl and extracted with 50% EtOAc in hexanes (15 mL×3). The combined organic layers were dried over Na₂SO₄, filtered off and filtrated concentrated in vacuo and the residue purified using flash silica gel column chromatography 5%-8% MeOH in DCM with eluent containing 1% TEA. Product details; 46 mg, 88% as a white solid of TEA salt.

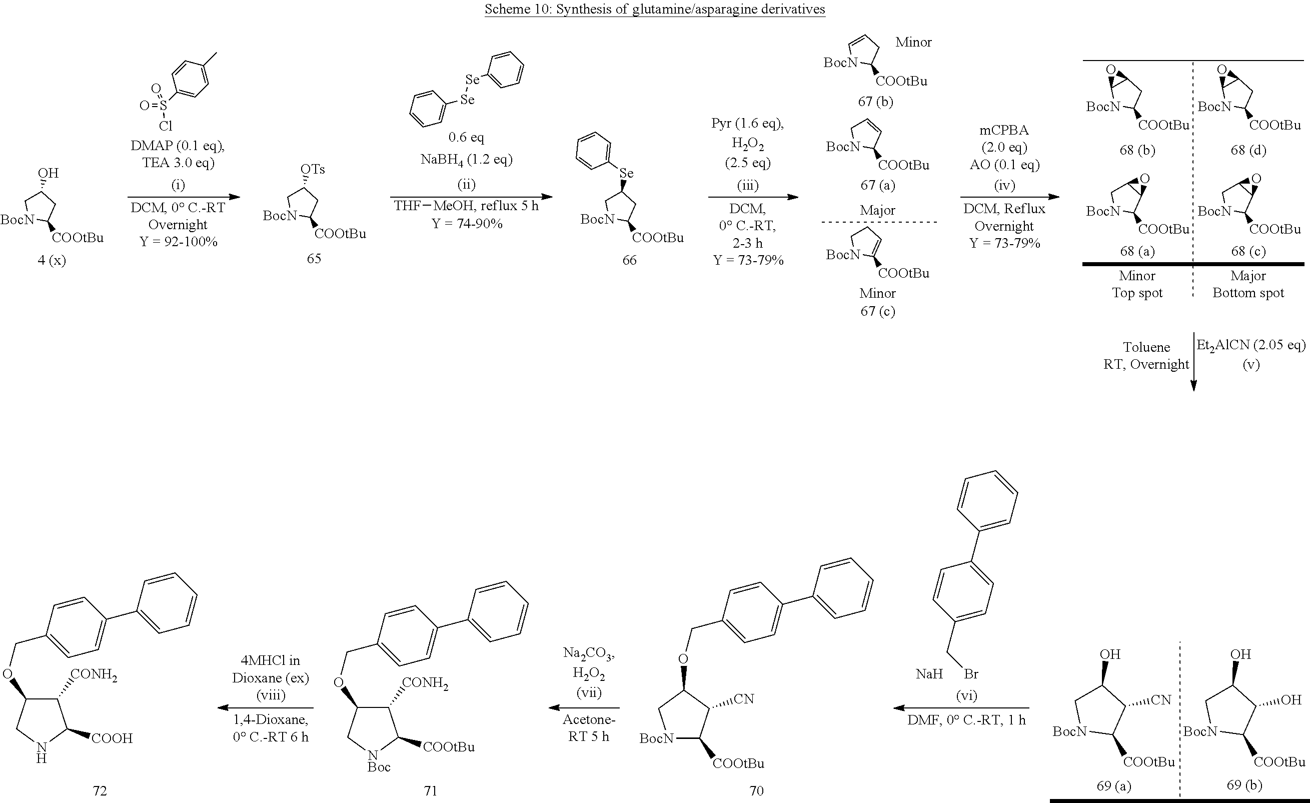

### Procedures for Scheme 10 Intermediates

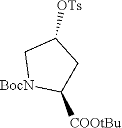

di-tert-butyl (2S,4R)-4-(tosyloxy)pyrrolidine-1,2-dicarboxylate, 65. Scheme 10 step (i); 4 (x) (1000 mg, 3.48 mmol), TEA (1.5 mL, 10.44 mmol) and DMAP (43 mg, 0.35 mmol) were dissolved in DCM (6.0 mL) and solution cooled to 0° C. Toluene sulfonyl chloride (191 mg, 4.18 mmol) in DCM (˜3.0 mL) was added slowly and reaction was let to warm up to room temperature and stirred overnight. Excess DCM was removed in vacuo and residue taken up in 20 mL of 50% EtOAc in hexanes and transferred into separating funnel. Organic layer was washed with NaHCO₃ (2×10 mL), chilled 1M HCl (2×10 mL), and 4:1 mixture of brine and water (2×10 mL). The organic layer was dried over Na₂SO₄, filtered off and filtrated concentrated in vacuo and the residue purified using flash silica gel column chromatography 15%-40% EtOAc in hexanes. Product; 1.4208 g, 92.4% as a colorless oil.

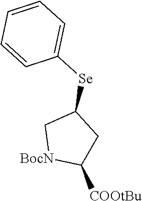

di-tert-butyl (2S,4S)-4-(phenylselanyl)pyrrolidine-1,2-dicarboxylate, 66. Scheme 9 step (ii); Diphenyl diselenide (0.668 g, 2.14 mmol) and NaBH4 (150 mg, 4.00 mmol) were charged into a three-necked-flask and 1:1 mixture of THE and MeOH added (16 mL) added under N₂. The solution was then refluxed at 80° C. for 20 minutes. 65 (1.35 g, 3.06 mmol) in THE (8 mL) was added dropwise at room temperature Reaction was brought to reflux again for 7 hours. 0.200 g of NaBH4 was added in two portions and reaction stirred for an additional 12 hours. Reaction was quenched with water and contents diluted with 50 mL 1:1 mixture of EtOAc and hexanes and transferred into separating funnel. Aqueous layer was washed with water (3×50 mL) and the organic layer dried over Na₂SO₄, filtered off and filtrated concentrated in vacuo and the residue purified using flash silica gel column chromatography 0%-25% EtOAc in hexanes. Product; 1.17 g, 90% as a colorless oil.

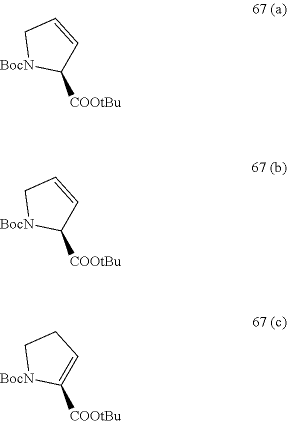

di-tert-butyl (S)-2,5-dihydro-1H-pyrrole-1,2-dicarboxylate, 67. Scheme 10 step (iii); 55 (750 mg, 1.95 mmol) and pyridine (0.25 mL, 3.12 mmol) were dissolved in DCM and solution cooled to 0° C. 30% H₂O₂ was added dropwise and reaction stirred for 2 hours at room temperature until 100% conversion on TLC. Reaction mixture was transferred into separating funnel and washed with chilled 1M HCl (3×10 mL), saturated Na₂SO₃ (3×10 mL) and 1:1 mixture of brine and water (2×10 mL). Organic layer dried over Na₂SO₄, filtered off and filtrated concentrated in vacuo and the residue purified using flash silica gel column chromatography 0%-40% EtOAc in hexanes to give three major possible isomers, 67 (a) as major isomer, 67 (b) and 67 (c) were also detected. 67 (c) was separated out. Product, mixture of 67 (a) and 67 (b); 530 mg, 73% as a clear pale-yellow oil.

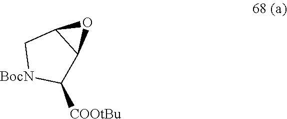

di-tert-butyl (1R,2S,5S)-6-oxa-3-azabicyclo[3.1.0]hexane-2,3-dicarboxylate, 68 (a). Scheme 10 step (iv); 67 (a) (453 mg, 1.68 mmol), anti-oxidant (4,4′-Thiobis(6-tert-butyl-m-cresol), AO (60 mg, 0.17 mmol) and mCPBA (581 mg, 3.36 mmol) were dissolved in 10 mL DCM under N₂ then refluxed (50° C.) for 16 hours. Additional mCPBA (377 mg) and anti-oxidant (60 mg) were added and refluxed for an additional 24 hours. Reaction mixture was then cooled to room temperature then to 0° C. The white precipitate was filtered off and washed with chilled DCM. The filtrate was evaporated under reduced pressure. The residue was taken up in ether and transferred into separating funnel, washed with NaHCO₃ (5×10 mL) and 1:1 brine water mixture (2×10 mL). Ether layer was dried over Na₂SO₄, filtered off and filtrated concentrated in vacuo (purification is pending).

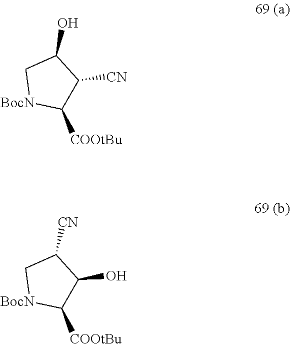

di-tert-butyl (2S,3S,4S)-4-cyano-3-hydroxypyrrolidine-1,2-dicarboxylate, 69 (a). (Protocol to be followed) According to Scheme 10 step (v); Et₂AlCN (2.0 eq) in toluene is slowly added to a solution of 68 (a) (1.0 eq) in toluene at room temperature and stirred overnight. Reaction is quenched with 1. ON NaOH and diluted with 10 mL water. Aqueous layer is extracted twice with ethyl acetate (2×60 mL). Combined organic layers are washed with water and brine (2×60 mL) and dried over Na₂SO₄ then filtered and concentrated under reduced pressure to yield 69 (a) plus an isomer 69 (b) to be used in the next step after purification.

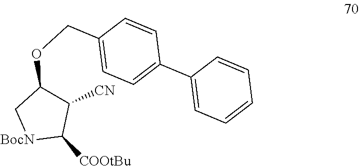

di-tert-butyl (2S,3S,4S)-3-([1,1′-biphenyl]-4-ylmethoxy)-4-cyanopyrrolidine-1,2-dicarboxylate, 70. To be synthesized according to general procedure for Williamson ether synthesis (to be synthesized same as SN 39).

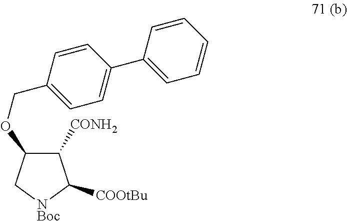

1-(tert-butyl) 2-methyl (2S,3S,4R)-3-([1,1′-biphenyl]-4-ylmethoxy)-4-carbamoylpyrrolidine-1,2-dicarboxylate, 71. Typical procedure; 71 (1.0 eq) is dissolved in acetone and 1N aqueous of Na₂CO₃ (3.0) added. 30% of H₂O₂ (27 eq) is added is added and mixture stirred at room temperature until completion. Excess solvent is removed under reduced pressure. The residue is taken up in EtOAc and washed with water. The water layer is washed three times with EtOAc. Combined organic layers are dried over Na₂SO₄ then filtered, filtrate concentrated under reduced pressure to give 71 as pure solid.

Compound #SN 56

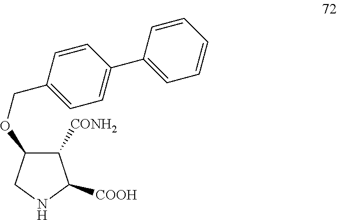 [SMILES: NC(=O)[C@@H]1[C@@H](C(=O)O)NC[C@H]1OCc1ccc(-c2ccccc2)cc1]

(2S,3S,4R)-3-([1,1′-biphenyl]-4-ylmethoxy)-4-carbamoylpyrrolidine-2-carboxylic acid, 72. Typical procedure for deprotection. Protected amide 71 (1.0 equivalent) is dissolved in 2.0 mL of dry 1,4-dioxane and cooled to 0° C. 4M HCl (excess) in 1,4 dioxane is added dropwise under N₂. The reaction is left to warm up to room temperature and stirred for 24 hours then sonicated for an additional 2 hours. Excess Dioxane is removed in vacuo by repeatedly adding DCM (2×3 mL) and evaporating under reduced pressure until a white powder forms. The powder is suspended in 1 mL of DCM and 3 mL of EtOAc and sonicated for 30 minutes and filtered off to yield analytically pure solid 72.

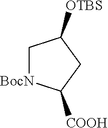 [SMILES: CC(C)(C)OC(=O)N1C[C@@H](O[Si](C)(C)C(C)(C)C)C[C@H]1C(=O)O]

(2S,4S)-1-(tert-butoxycarbonyl)-4-((tert-butyldimethylsilyl)oxy)pyrrolidine-2-carboxylic acid 2 (i). Prepared according to general procedure for step (i). In brief, (2S,4S)-1-(tert-butoxycarbonyl)-4-hydroxypyrrolidine-2-carboxylic acid (1) (500 mg, 2.16 mmol) and imidazole (744 mg, 10.80 mmol) were weighed into an oven-dried round bottomed flask (RBF) and dissolved in dry DMF (6 mL). The reaction mixture was cooled to 0° C. and TBSCI in dry DMF (652 mg, 4.32 mmol) was added dropwise under N₂ gas. See full protocol (general procedure step (i). Product details; 597 mg, 1.73 mmol, 80% yield as white powder.

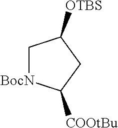

di-tert-butyl (2S,4S)-4-((tert-butyldimethylsilyl)oxy)pyrrolidine-1,2-dicarboxylate 3 (i). Prepared according to general procedure step (ii). 2 (i) (661 mg, 1.91 mmol) was dissolved in dry DCM and cooled to 0° C. 1.03 mL of tert-Butyl 2,2,2-trichloroacetimidate (1.252 mg, 5.73 mmol) was added dropwise under N₂ and the mixture was stirred for 36 hours at ambient temperatures. Product details; 690 mg, 90% yield as a colorless oil.

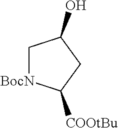

di-tert-butyl (2S,4S)-4-hydroxypyrrolidine-1,2-dicarboxylate 4 (iii). Prepared according to general procedure step (iii). 3 (i) (635 mg, 1.58 mmol) dry THE (6 mL) and 1M TBAF in THE (2.05 mL, 2.05 mmol). Product details; 449 mg, 1.56 mmol, 99% yield as colorless oil, turns solid on freezing.

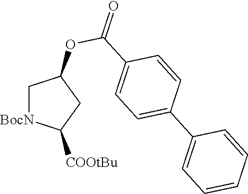

di-tert-butyl (2S,4R)-4-(([1,1′-biphenyl]-4-carbonyl)oxy)pyrrolidine-1,2-dicarboxylate 7 (a). Synthesized according to the general Steglich protocol for esters, scheme 1 step (iv). 4 (iii) (135 mg, 0.47 mmol), DMAP (5.7 mg, 0.05 mmol), [1,1′-biphenyl]-4-carboxylic acid (279 mg, 1.41 mmol) and DCC (116, 0.56 mmol). Product details; 205 mg, 94% as a colorless oil, turns to white solid on freezing.

Compound #SN 05

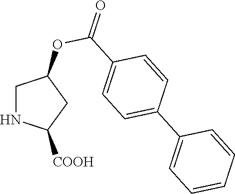 [SMILES: O=C(O[C@@H]1CN[C@H](C(=O)O)C1)c1ccc(-c2ccccc2)cc1]

Prepared according to general TFA deprotection procedure scheme 1 step (v). 7 (a) (140 mg, 0.30 mmol), DTT (94 mg, 0.60 mmol) and TFA (0.73 mL, 9.60 mmol). Product details; 60 mg, 47% as a light-pinkish solid. In some embodiments, SN 05 is not claimed.

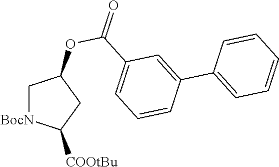

di-tert-butyl (2S,4S)-4-(([1,1′-biphenyl]-3-carbonyl)oxy)pyrrolidine-1,2-dicarboxylate 7 (b). Synthesized according to the general Steglich protocol for esters, scheme 1 step (iv). 4 (iii) (53 mg, 0.18 mmol), DMAP (4.5 mg, 0.04 mmol), biphenyl-3-carboxylic acid (110 mg, 0.55 mmol) and DCC (42, 0.20 mmol). Product details; 29 mg, 34% as a colorless oil, turns to white solid after freezing overnight.

Compound #SN 06

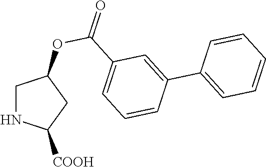 [SMILES: O=C(O[C@@H]1CN[C@H](C(=O)O)C1)c1cccc(-c2ccccc2)c1]

Prepared according to general TFA deprotection procedure scheme 1 step (v). 7 (b) (29 mg, 0.06 mmol), DTT (19 mg, 0.12 mmol) and TFA (0.20 mL, 2.53 mmol). Product details; 8 mg, 31% as a white precipitate.

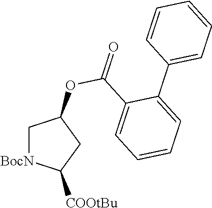

di-tert-butyl (2S,4S)-4-(([1,1′-biphenyl]-2-carbonyl)oxy)pyrrolidine-1,2-dicarboxylate 7 (c). Synthesized according to Mitsunobu esterification protocol. 4 (iv) (100 mg, 0.35 mmol), biphenyl-2-carboxylic acid (138 mg, 0.70 mmol) and diphenyl(4-pyridyl)phosphine (183 mg, 0.70 mmol) were dissolved in dry THF (1.0 mL) and cooled to 0° C. TEA (0.1 mL, 0.70 mmol) and DIAD (0.15 mL, 0.70 mmol) added under N₂ atmosphere. The reaction was left to warm up to room temperature and stirred for 24 hours monitored by TLC. Reaction mixture was transferred into separating funnel and diluted with EtOAc and washed with 0.5M HCl (10 mL×3), NaHCO₃ (10 mL×3) and brine (5 mL×3). The organic layer was dried over Na₂SO₄ and concentrated in vacuo. The residue was purified using silica gel flash chromatography using 0-25% EtOAc in hexanes to obtain a pure colorless oil (37 mg, 23%) and impure fraction 120 mg (not used). Pure fraction was used in the next step.

Compound #SN 07

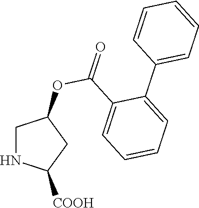 [SMILES: O=C(O[C@@H]1CN[C@H](C(=O)O)C1)c1ccccc1-c1ccccc1]

Prepared according to general TFA deprotection procedure scheme 1 step (v). 7 (b) (37 mg, 0.08 mmol), DTT (25 mg, 0.16 mmol) and TFA (0.2 mL, 0.19 mmol). Product details; 20 mg, 60% as a white precipitate.

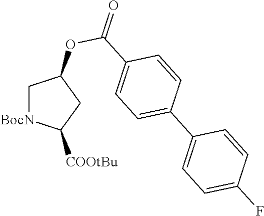

di-tert-butyl (2S,4S)-4-((4′-fluoro-[1,1′-biphenyl]-4-carbonyl)oxy)pyrrolidine-1,2-dicarboxylate 7 (d). Synthesized according to the general Steglich protocol for esters, scheme 1 step (iv). 4 (iii) (40 mg, 0.14 mmol), DMAP (2 mg, 0.01 mmol), 4′-fluoro-[1,1′-biphenyl]-4-carboxylic acid (90 mg, 0.42 mmol) and DCC (43, 0.21 mmol). Product details; 60 mg, 89% as a white solid.

Compound #SN 08

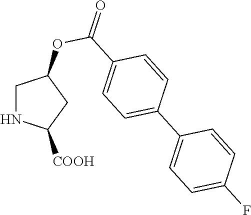 [SMILES: O=C(O[C@@H]1CN[C@H](C(=O)O)C1)c1ccc(-c2ccc(F)cc2)cc1]

Prepared according to general TFA deprotection procedure scheme 1 step (v). 7 (d) (52 mg, 0.11 mmol), DTT (33 mg, 0.21 mmol) and TFA (1.0 mL, excess). Product details; 35 mg, 74% as an off-white powder.

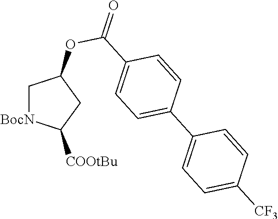

di-tert-butyl (2S,4S)-4-((4′-(trifluoromethyl)-[1,1′-biphenyl]-4-carbonyl)oxy)pyrrolidine-1,2-dicarboxylate 7 (e). Synthesized according to the general Steglich protocol for esters, scheme 1 step (iv). 4 (iii) (35 mg, 0.12 mmol), DMAP (3 mg, 0.02 mmol), 4′-(trifluoromethyl)-[1,1′-biphenyl]-4-carboxylic acid (130 mg, 0.49 mmol) and DCC (50, 0.24 mmol). Product details; 58 mg, 88% as a white solid.

Compound #SN 09

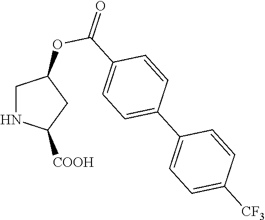 [SMILES: O=C(O[C@@H]1CN[C@H](C(=O)O)C1)c1ccc(-c2ccc(C(F)(F)F)cc2)cc1]

Prepared according to general TFA deprotection procedure scheme 1 step (v). 7 (d) (58 mg, 0.11 mmol), DTT (33 mg, 0.22 mmol) and TFA (1.0 mL, excess). Product details; 40 mg, 75% as an off-white powder.

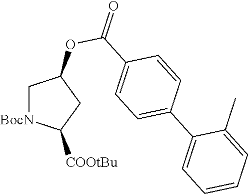

di-tert-butyl (2S,4S)-4-((2′-methyl-[1,1′-biphenyl]-4-carbonyl)oxy)pyrrolidine-1,2-dicarboxylate 7 (f). Synthesized according to the general Steglich protocol for esters, scheme 1 step (iv). 4 (iii) (48 mg, 0.17 mmol), DMAP (2 mg, 0.02 mmol), 2′-methyl-[1,1′-biphenyl]-4-carboxylic acid (106 mg, 0.50 mmol) and DCC (52, 0.25 mmol). Product details; 80 mg, 99% as a white solid.

Compound #SN 10

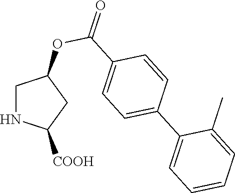 [SMILES: Cc1ccccc1-c1ccc(C(=O)O[C@@H]2CN[C@H](C(=O)O)C2)cc1]

Prepared according to general TFA deprotection procedure scheme 1 step (v). 7 (f) (80 mg, 0.17 mmol), DTT (51 mg, 0.33 mmol) and TFA (1.0 mL, excess). Product details; 45 mg, 62% as an off-white powder.

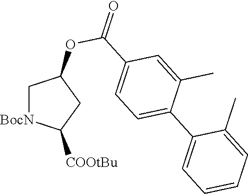

di-tert-butyl (2S,4S)-4-((2,2′-dimethyl-[1,1′-biphenyl]-4-carbonyl)oxy)pyrrolidine-1,2-dicarboxylate 7 (g). Synthesized according to the general Steglich protocol for esters, scheme 1 step (iv). 4 (iii) (75 mg, 0.26 mmol), DMAP (6.4 mg, 0.05 mmol), 2,2′-dimethyl-[1,1′-biphenyl]-4-carboxylic acid (177 mg, 0.78 mmol) and DCC (64, 0.31 mmol). Product details; 101 mg, 78% as a colorless oil, turns to white solid after freezing.

Compound #SN 11

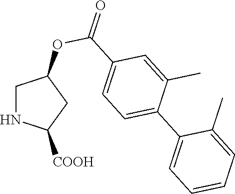 [SMILES: Cc1ccccc1-c1ccc(C(=O)O[C@@H]2CN[C@H](C(=O)O)C2)cc1C]

Prepared according to general TFA deprotection procedure scheme 1 step (v). 7 (g) (71 mg, 0.14 mmol), DTT (44 mg, 0.28 mmol) and TFA (0.45 mL, 4.58 mmol). Product details; 32 mg, 50% as an off-white powder.

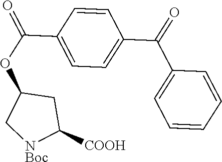 [SMILES: CC(C)(C)OC(=O)N1C[C@@H](OC(=O)c2ccc(C(=O)c3ccccc3)cc2)C[C@H]1C(=O)O]

di-tert-butyl (2S,4S)-4-((2,2′-dimethyl-[1,1′-biphenyl]-4-carbonyl)oxy)pyrrolidine-1,2-dicarboxylate 7 (h). Synthesized according to the general Steglich protocol for esters, scheme 1 step (iv). 4 (iii) (80 mg, 0.28 mmol), DMAP (6.8 mg, 0.06 mmol), 4-benzoylbenzoic acid (189 mg, 0.84 mmol) and DCC (63, 0.31 mmol). Product details; 120 mg, 87% as a white solid.

Compound #SN 12

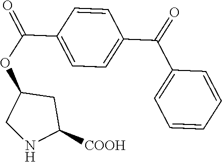 [SMILES: O=C(O[C@@H]1CN[C@H](C(=O)O)C1)c1ccc(C(=O)c2ccccc2)cc1]

Prepared according to general TFA deprotection procedure scheme 1 step (v). 7 (h) (116 mg, 0.23 mmol), DTT (72 mg, 0.46 mmol) and TFA (0.60 mL, 7.36 mmol). Product details; 73 mg, 69% as an off-white powder.

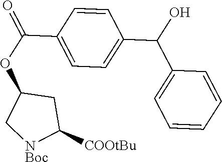

di-tert-butyl (2S,4S)-4-((4-(hydroxy(phenyl)methyl)benzoyl)oxy)pyrrolidine-1,2-dicarboxylate, 7 (h-i). 7 (h) (47 mg, 0.09 mmol) was dissolved in DCM (0.5 mL)/methanol 1.0 mL mixture and cooled to 0° C. NaBH4 (7.1 mg, 0.19 mmol) was added in one portion. The reaction was let to warm up to room temperature and stirred for 2 hours until complete conversion as monitored by TLC. Reaction was quenched by addition of acetone and transferred into separating funnel and 5.0 mL of water added then product extracted with DCM (10 mL×3). Organic layer was dried over Na₂SO₄ and filtered off. Filtrate was concentrated in vacuo and residue purified using silica gel flash chromatography to yield 35 mg, 75% as a white solid.

Compound #SN 13

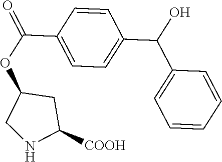 [SMILES: O=C(O[C@@H]1CN[C@H](C(=O)O)C1)c1ccc(C(O)c2ccccc2)cc1]

Prepared according to general TFA deprotection procedure scheme 1 step (v). 7 (h-i) (35 mg, 0.07 mmol), DTT (22 mg, 0.14 mmol) and TFA (0.2 mL, 2.25 mmol). Product details; 8 mg, 25% as an off-white powder.

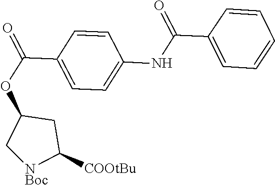

di-tert-butyl (2S,4S)-4-((4-benzamidobenzoyl)oxy)pyrrolidine-1,2-dicarboxylate 7 (i). Synthesized according to the general Steglich protocol for esters, scheme 1 step (iv). 4 (iii) (65 mg, 0.23 mmol), DMAP (14 mg, 0.11 mmol), 4-benzamidobenzoic acid (218 mg, 0.91 mmol) and DCC (103, 0.49 mmol). Product details; 74 mg, 64% white solid.

Compound #SN 14

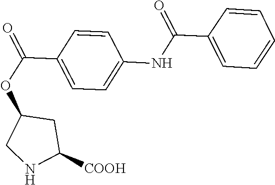 [SMILES: O=C(Nc1ccc(C(=O)O[C@@H]2CN[C@H](C(=O)O)C2)cc1)c1ccccc1]

Prepared according to general TFA deprotection procedure scheme 1 step (v). 7 (i) (70 mg, 0.14 mmol), DTT (42 mg, 0.27 mmol) and TFA (0.40 mL, 4.38 mmol). Product details; 40 mg, 61% off-white powder.

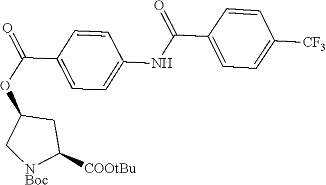

di-tert-butyl (2S,4S)-4-((4-benzamidobenzoyl)oxy)pyrrolidine-1,2-dicarboxylate 7 (j). Synthesized according to the general Steglich protocol for esters, scheme 1 step (iv). 4 (iii) (54 mg, 0.20 mmol), DMAP (2.5 mg, 0.02 mmol), (4′-Trifluoromethyl)-4-benzamidobenzoic acid (187 mg, 0.60 mmol) and DCC (46, 0.22 mmol). Product details; 48 mg, 41% as colorless oil, white solid on freezing.

Compound #SN 15

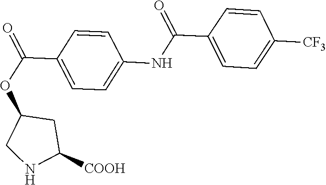 [SMILES: O=C(Nc1ccc(C(=O)O[C@@H]2CN[C@H](C(=O)O)C2)cc1)c1ccc(C(F)(F)F)cc1]

Prepared according to general TFA deprotection procedure scheme 1 step (v). 7 (j) (48 mg, 0.08 mmol), DTT (26 mg, 0.17 mmol) and TFA (0.20 mL, 2.66 mmol). Product details; 17 mg, 38% off-white powder.

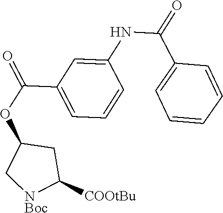

di-tert-butyl (2S,4S)-4-((3-benzamidobenzoyl)oxy)pyrrolidine-1,2-dicarboxylate 7 (k). Synthesized according to the general Steglich protocol for esters, scheme 1 step (iv). 4 (iii) (80 mg, 0.28 mmol), DMAP (17 mg, 0.14 mmol), 3-benzamidobenzoic acid (268 mg, 1.11 mmol) and DCC (86, 0.42 mmol). Product details; 120 mg, 84% white solid.

Compound #SN 16

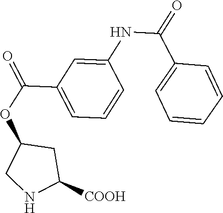 [SMILES: O=C(Nc1cccc(C(=O)O[C@@H]2CN[C@H](C(=O)O)C2)c1)c1ccccc1]

Prepared according to general TFA deprotection procedure scheme 1 step (v). 7 (k) (38 mg, 0.07 mmol), DTT (23 mg, 0.15 mmol) and TFA (0.18 mL, 2.38 mmol). Product details; 20 mg, 58% white powder.

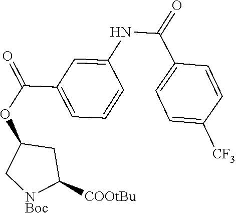

di-tert-butyl (2S,4S)-4-((3-(4-(trifluoromethyl)benzamido)benzoyl)oxy)pyrrolidine-1,2-dicarboxylate 7 (1). Synthesized according to the general Steglich protocol for esters, scheme 1 step (iv). 4 (iii) (80 mg, 0.28 mmol), DMAP (17 mg, 0.14 mmol), (4′-Trifluomethyl)-3-benzamidobenzoic acid (344 mg, 1.11 mmol) and DCC (86, 0.42 mmol). Product details; 130 mg, 80%, white solid.

Compound #SN 17

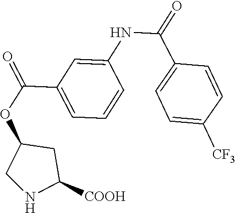 [SMILES: O=C(Nc1cccc(C(=O)O[C@@H]2CN[C@H](C(=O)O)C2)c1)c1ccc(C(F)(F)F)cc1]

Prepared according to general TFA deprotection procedure scheme 1 step (v). 7 (1) (36 mg, 0.06 mmol), DTT (19 mg, 0.12 mmol) and TFA (0.15 mL, 2.00 mmol). Product details; 26 mg, 81% white powder.

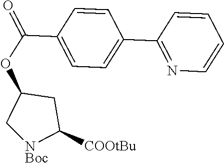

di-tert-butyl (2S,4S)-4-((4-(pyridin-2-yl)benzoyl)oxy)pyrrolidine-1,2-dicarboxylate 7 (m). Synthesized according to the general Steglich protocol for esters, scheme 1 step (iv). 4 (iii) (60 mg, 0.21 mmol), DMAP (13 mg, 0.10 mmol), 4-(pyridin-2-yl)benzoic acid (166 mg, 0.84 mmol) and DCC (65, 0.31 mmol), pyridine (0.5 mL) and DMF (0.5 mL). Procedure modified to enhance solubility of carboxylic acid. Product details; 75 mg, 76%, white solid.

Compound #SN 18

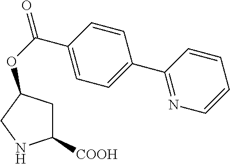 [SMILES: O=C(O[C@@H]1CN[C@H](C(=O)O)C1)c1ccc(-c2ccccn2)cc1]

Prepared according to general TFA deprotection procedure scheme 1 step (v). 7 (m) (68 mg, 0.15 mmol), DTT (45 mg, 0.29 mmol) and TFA (0.36 mL, 4.64 mmol). Product details; 48 mg, 78%, white powder.

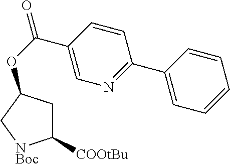

di-tert-butyl (2S,4S)-4-((6-phenylnicotinoyl)oxy)pyrrolidine-1,2-dicarboxylate 7 (n). Synthesized according to the general Steglich protocol for esters, scheme 1 step (iv). 4 (iii) (65 mg, 0.23 mmol), DMAP 14 mg, 0.11 mmol), 6-phenylnicotinic acid (180 mg, 0.91 mmol) and DCC (70, 0.34 mmol) and TEA (1 mL). Procedure modified to enhance solubility of carboxylic acid. Product details; 69 mg, 64%, white solid.

Compound #SN 19

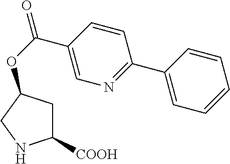 [SMILES: O=C(O[C@@H]1CN[C@H](C(=O)O)C1)c1ccc(-c2ccccc2)nc1]

Prepared according to general TFA deprotection procedure scheme 1 step (v). 7 (n) (71 mg, 0.15 mmol), DTT (47 mg, 0.30 mmol) and TFA (0.37 mL, 4.86 mmol). Product details; 49 mg, 76% as a white powder.

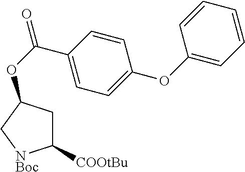

di-tert-butyl (2S,4S)-4-((4-phenoxybenzoyl)oxy)pyrrolidine-1,2-dicarboxylate 7 (o). Synthesized according to the general Steglich protocol for esters, scheme 1 step (iv). 4 (iii) (90 mg, 0.31 mmol), DMAP 19 mg, 0.16 mmol), 4-phenoxybenzoic acid (302 mg, 1.41 mmol) and DCC (97, 0.47 mmol). Product details; 128 mg, 85%, white solid.

Compound #SN 20

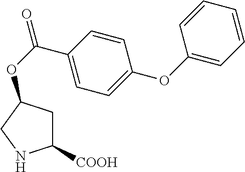 [SMILES: O=C(O[C@@H]1CN[C@H](C(=O)O)C1)c1ccc(Oc2ccccc2)cc1]

Prepared according to general TFA deprotection procedure scheme 1 step (v). 7 (o) (57 mg, 0.12 mmol), DTT (36 mg, 0.24 mmol) and TFA (0.30 mL, 3.77 mmol). Product details; 20 mg, 39% as a white solid.

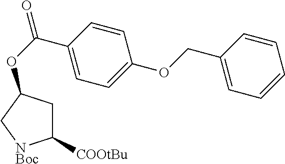

di-tert-butyl (2S,4S)-4-((4-(benzyloxy)benzoyl)oxy)pyrrolidine-1,2-dicarboxylate 7 (p). Synthesized according to the general Steglich protocol for esters, scheme 1 step (iv). 4 (iii) (160 mg, 0.56 mmol), DMAP 34 mg, 0.28 mmol), 4-(benzyloxy)benzoic acid (381 mg, 1.67 mmol) and DCC (126, 0.61 mmol) and TEA (0.16 mL, 2 eq). Procedure modified to enhance solubility of carboxylic acid. Product details; 277 mg, 100%, white solid.

Compound #SN 21

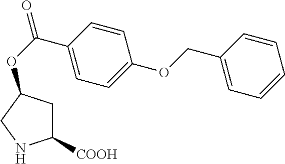 [SMILES: O=C(O[C@@H]1CN[C@H](C(=O)O)C1)c1ccc(OCc2ccccc2)cc1]

Prepared according to general TFA deprotection procedure scheme 1 step (v). 7 (p) (250 mg, 0.50 mmol), No DTT used, TFA (2.75 mL, excess). Product details; 127 mg, 56% as a light brown powder.

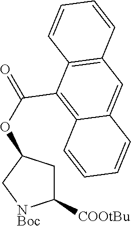

di-tert-butyl (2S,4S)-4-((anthracene-9-carbonyl)oxy)pyrrolidine-1,2-dicarboxylate 7 (q). Synthesized according to the general Steglich protocol for esters, scheme 1 step (iv). 4 (iii) (85 mg, 0.30 mmol), DMAP (7.2 mg, 0.06 mmol), 9-anthracene carboxylic acid (197 mg, 0.89 mmol) and DCC (73, 0.35 mmol). Product details; 34 mg, 23%, yellow solid.

Compound #SN 22

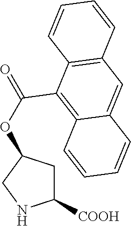 [SMILES: O=C(O[C@@H]1CN[C@H](C(=O)O)C1)c1c2ccccc2cc2ccccc12]

Prepared according to general TFA deprotection procedure scheme 1 step (v). 7 (q) (34 mg, 0.07 mmol), DTT (21 mg, 0.14 mmol) and TFA (0.15 mL, 2.19 mmol). Product details; 5 mg, 16% as a yellow powder.

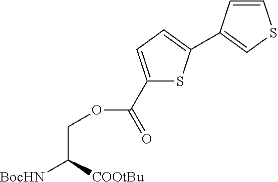

(S)-3-(tert-butoxy)-2-((tert-butoxycarbonyl)amino)-3-oxopropyl [2,3′-bithiophene]-5-carboxylate 9 (a). Synthesized according to the general Steglich protocol for esters, scheme 2 step (iv). 4 (v) (120 mg, 0.46 mmol), DMAP (5.6 mg, 0.05 mmol), [2,2′-bithiophene]-5-carboxylic acid (290 mg, 1.38 mmol) and DCC (104, 0.46 mmol). Product details; 108 mg, 52%, yellow solid.

Compound #SN 23

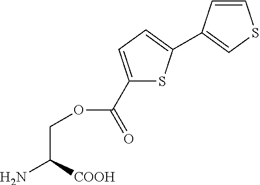 [SMILES: N[C@@H](COC(=O)c1ccc(-c2ccsc2)s1)C(=O)O]

Prepared according to general TFA deprotection procedure scheme 2 step (v). 7 (p) (60 mg, 0.13 mmol), DTT (41 mg, 0.26 mmol) and TFA (0.32 mL, 4.23 mmol). Product details; 20 mg, 37% as a yellow powder.

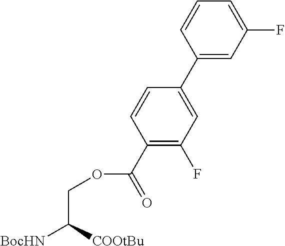

(S)-3-(tert-butoxy)-2-((tert-butoxycarbonyl)amino)-3-oxopropyl 3,3′-difluoro-[1,1′-biphenyl]-4-carboxylate 9 (b). Synthesized according to the general Steglich protocol for esters, scheme 2 step (iv). 4 (v) (109 mg, 0.42 mmol), DMAP (5.1 mg, 0.04 mmol), 3,3′-difluoro-[1,1′-biphenyl]-4-carboxylic acid (295 mg, 1.26 mmol) and DCC (95, 0.46 mmol). Product details; 94 mg, 47%, colorless oil.

Compound #SN 24

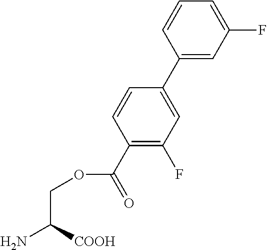 [SMILES: N[C@@H](COC(=O)c1ccc(-c2cccc(F)c2)cc1F)C(=O)O]

Prepared according to general TFA deprotection procedure scheme 2 step (v). 9 (b) (56 mg, 0.12 mmol), DTT (36 mg, 0.24 mmol) and TFA (0.29 mL, 3.71 mmol). Product details; 27 mg, 53%, white powder.

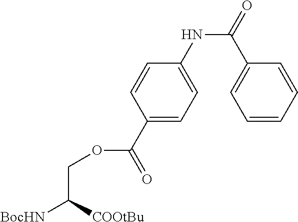

(S)-3-(tert-butoxy)-2-((tert-butoxycarbonyl)amino)-3-oxopropyl 4-benzamidobenzoate 9 (c). Synthesized according to the general Steglich protocol for esters, scheme 2 step (iv). 4 (v) (250 mg, 0.96 mmol), DMAP (58 mg, 0.48 mmol), 4-benzamidobenzoic acid (923 mg, 3.83 mmol), DCC (217, 1.05 mmol), TEA (0.50 mL, 4 eq) and DMF (0.5 mL). Protocol was modified to improve solubility of carboxylic acid. Product details; 250 mg, 54%, off-white powder.

Compound #SN 25

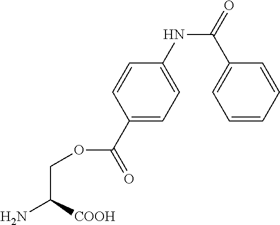 [SMILES: N[C@@H](COC(=O)c1ccc(NC(=O)c2ccccc2)cc1)C(=O)O]

Prepared according to general TFA deprotection procedure scheme 2 step (v). 9 (c) (140 mg, 0.29 mmol), and TFA (2.0 mL, excess). DTT was not used. Product details; 103 mg, 81%, off-white powder.

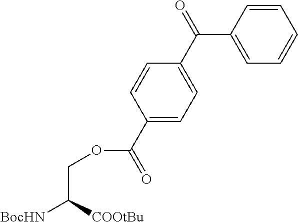

(S)-3-(tert-butoxy)-2-((tert-butoxycarbonyl)amino)-3-oxopropyl 4-benzoylbenzoate 9 (d). Synthesized according to the general Steglich protocol for esters, scheme 2 step (iv). 4 (v) (120 mg, 0.46 mmol), DMAP (5.6 mg, 0.05 mmol), 4-benzoylbenzoic acid (312 mg, 1.38 mmol) and DCC (95, 0.46 mmol). Product details; 214 mg, 99%, white solid.

Compound #SN 26,

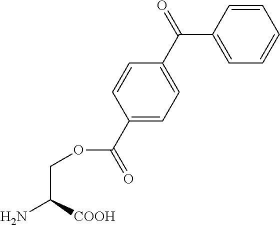 [SMILES: N[C@@H](COC(=O)c1ccc(C(=O)c2ccccc2)cc1)C(=O)O]

Prepared according to general TFA deprotection procedure scheme 2 step (v). 9 (d) (90 mg, 0.19 mmol), DTT (59 mg, 0.38 mmol) and TFA (0.47 mL, 6.08 mmol). Product details; 60 mg, 74%, white solid.

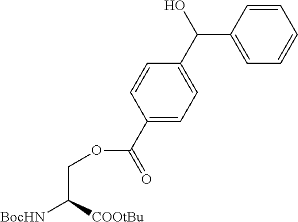

(S)-3-(tert-butoxy)-2-((tert-butoxycarbonyl)amino)-3-oxopropyl 4-(hydroxy(phenyl)methyl)benzoate 9 (d-i). 9 (d) (40 mg, 0.09 mmol) was dissolved in DCM (0.5 mL)/methanol 1.0 mL mixture and cooled to 0° C. NaBH4 (6.4 mg, 0.17 mmol) was added in one portion. The reaction was let to warm up to room temperature and stirred for 2 hours until complete conversion as monitored by TLC. Reaction was quenched by addition of acetone and transferred into separating funnel and 5.0 mL of water added then product extracted with DCM (10 mL×3). Organic layer was dried over Na₂SO₄ and filtered off. Filtrate was concentrated in vacuo and residue purified using silica gel flash chromatography to yield 40 mg, 99% as a white solid.

Compound #SN 27

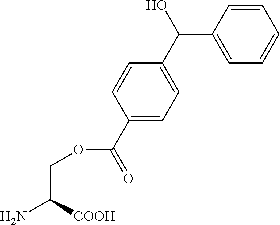 [SMILES: N[C@@H](COC(=O)c1ccc(C(O)c2ccccc2)cc1)C(=O)O]

Prepared according to general TFA deprotection procedure scheme 2 step (v). 9 (d-i) (40 mg, 0.09 mmol), PhS (20 mg, 0.18 mmol) and TFA (0.21 mL, 2.72 mmol). Product details; 23 mg, 60%, off-white solid.

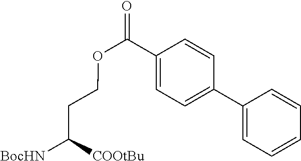

(S)-4-(tert-butoxy)-3-((tert-butoxycarbonyl)amino)-4-oxobutyl [1,1′-biphenyl]-4-carboxylate 29. Synthesized according to the general Steglich protocol for esters scheme 3 step (d). 28 (76 mg, 0.28 mmol), DMAP (6.7 mg, 0.06 mmol), 4-biphenyl carboxylic acid (219 mg, 1.10 mmol) and DCC (68 mg, 0.33 mmol). Product details; 61 mg, 48%, colorless oil which turns to white solid on cooling.

Compound #SN 33

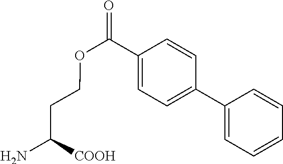 [SMILES: N[C@@H](CCOC(=O)c1ccc(-c2ccccc2)cc1)C(=O)O]

Prepared according to general TFA deprotection procedure scheme 3 step (e). 29 (60 mg, 0.13 mmol), DTT (41 mg, 0.26 mmol) and TFA (0.32 mL, 4.22 mmol). Product details; 36 mg, 66%, collected as a white powder.

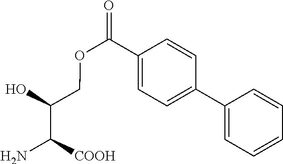 [SMILES: N[C@H](C(=O)O)[C@@H](O)COC(=O)c1ccc(-c2ccccc2)cc1]

(2R,3S)-4-(tert-butoxy)-3-((tert-butoxycarbonyl)amino)-2-hydroxy-4-oxobutyl [1,1′-biphenyl]-4-carboxylate 41. Synthesized according to the general protocol for ester synthesis using acyl chlorides as 9 (h) and 9 (i). 35 Diol (65 mg, 0.22 mmol), pyridine (0.05 mL, 0.67 mmol), DMAP (6.8 mg, 0.06 mmol), and biphenyl-4-carbonyl chloride (51 mg, 0.23 mmol). Product details; 32 mg, 31%, colorless oil which turns to white solid on cooling.

Compound #SN 34

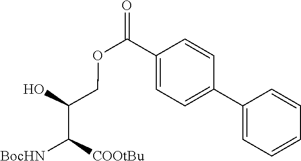

Prepared according to general TFA deprotection procedure. 41 (32 mg, 0.07 mmol), DTT (21 mg, 0.14 mmol) and TFA (0.17 mL, 2.17 mmol). Product details; 18 mg, 62%, collected as a white powder.

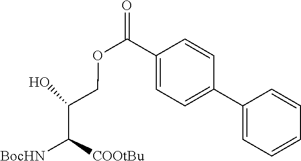

(2S,3S)-4-(tert-butoxy)-3-((tert-butoxycarbonyl)amino)-2-hydroxy-4-oxobutyl [1,1′-biphenyl]-4-carboxylate 47. Synthesized according to the general protocol for ester synthesis using acyl chlorides as 9 (h) and 9 (i). 36 Diol (50 mg, 0.17 mmol), pyridine (5.0 mL, excess, used as a solvent), DMAP (6.3 mg, 0.05 mmol), and biphenyl-4-carbonyl chloride (41 mg, 0.19 mmol). Reaction was refluxed at 75° C.-80° C. for 36 hours. TLC showed about 50% conversion. Pyridine was removed by evaporation under reduced pressure before general work up was done. Product details; 47 mg, 57%, white solid.

Compound #SN 35

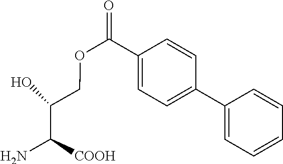 [SMILES: N[C@H](C(=O)O)[C@H](O)COC(=O)c1ccc(-c2ccccc2)cc1]

Prepared according to general TFA deprotection procedure. 47 (47 mg, 0.10 mmol), DTT (32 mg, 0.20 mmol) and TFA (0.25 mL, 3.27 mmol). Product details; 25 mg, 59%, collected as a white powder.

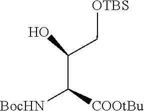

tert-butyl (2S,3R)-2-((tert-butoxycarbonyl)amino)-4-((tert-butyldimethylsilyl)oxy)-3-hydroxybutanoate 37.

35 (2S,3R) 225 mg, 0.77 mmol and imidazole (79 mg, 1.16 mmol) were dissolved in dry DCM (4.0 mL) and cooled to ice temperature under N₂. TBSCI (128 mg, 0.85 mmol) in dry DCM (2 mL) was added dropwise over a period of 10 minutes. Reaction was left to warm up to room temperature and stirred overnight (12 hours). TLC showed complete conversion. Reaction contents were transferred to separation funnel and diluted with DCM and washed once with 10 mL water and once with 10 mL brine. Organic layer was dried over sodium sulfate and filtered. Filtrate was concentrated in vacuo and residue purified using silica gel flash chromatography 0%-25% EtOAc in hexanes to yield a colorless oil, 312 mg, 100%.

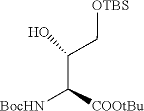

tert-butyl (2S,3S)-2-((tert-butoxycarbonyl)amino)-4-((tert-butyldimethylsilyl)oxy)-3-hydroxybutanoate 43.

Prepared same as 37 above. 36 (2S,3S) 60 mg, 0.21 mmol and imidazole (21 mg, 0.31 mmol) were dissolved in dry DCM (1.0 mL). TBSCI (34 mg, 0.23 mmol) in dry DCM (1 mL). Product details; Colorless oil, 76 mg, 93%.

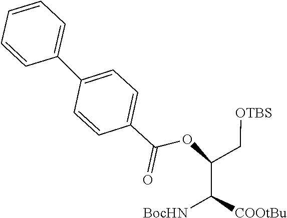

(6R,7S)-7-(tert-butoxycarbonyl)-2,2,3,3,11,11-hexamethyl-9-oxo-4,10-dioxa-8-aza-3-siladodecan-6-yl [1,1′-biphenyl]-4-carboxylate 38. Synthesized according to the general Steglich protocol for esters. 37 (74 mg, 0.18 mmol), DMAP (11 mg, 0.09 mmol), 4-biphenyl carboxylic acid (217 mg, 0.91 mmol (6eq)) and DCC (83, 0.40 mmol (2.2 eq), and DMF (0.5 mL). Procedure modified to enhance solubility of carboxylic acid, equivalents increased to raise reaction rate. Product details; 102 mg, 96%, colorless oil.

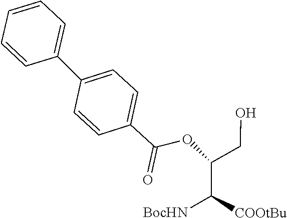

(6S,7S)-7-(tert-butoxycarbonyl)-2,2,3,3,11,11-hexamethyl-9-oxo-4,10-dioxa-8-aza-3-siladodecan-6-yl [1,1′-biphenyl]-4-carboxylate 44. Synthesized according to the general Steglich protocol for esters. 43 (41 mg, 0.10 mmol), DMAP (6.2 mg, 0.05 mmol), 4-biphenyl carboxylic acid (120 mg, 0.61 mmol (6eq)) and DCC (46, 0.22 mmol (2.2 eq)), and DMF (1.0 mL). Procedure modified to enhance solubility of carboxylic acid, equivalents increased to raise reaction rate. Product details; 59 mg, 100%, colorless oil.

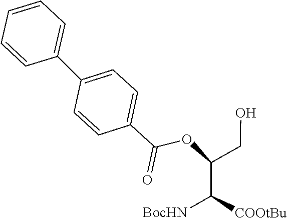

(2R,3S)-4-(tert-butoxy)-3-((tert-butoxycarbonyl)amino)-1-hydroxy-4-oxobutan-2-yl [1,1′-biphenyl]-4-carboxylate 39. Prepared according to general procedure scheme 1 step (iii). 38 (100 mg, 0.17 mmol) dry THE (1.5 mL) and 1M TBAF in THE (0.26 mL, 0.26 mmol). Product details; 78 mg, 97% yield as a colorless oil, turns solid on freezing.

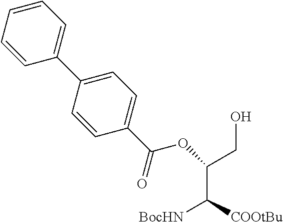

(2S,3S)-4-(tert-butoxy)-3-((tert-butoxycarbonyl)amino)-1-hydroxy-4-oxobutan-2-yl [1,1′-biphenyl]-4-carboxylate 45. Prepared according to general procedure scheme 1 step (iii). 44 (59 mg, 0.10 mmol) dry THE (2.0 mL) and 1M TBAF in THE (0.15 mL, 0.15 mmol). Product details; 48 mg, 100% yield as a colorless oil.

Compound #SN 36

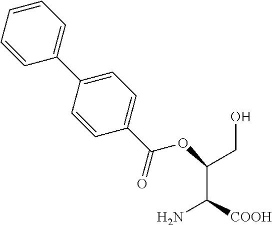 [SMILES: N[C@H](C(=O)O)[C@H](CO)OC(=O)c1ccc(-c2ccccc2)cc1]

Prepared according to general TFA deprotection procedure scheme 1 step (v). 39 (78 mg, 0.17 mmol), DTT (51 mg, 0.33 mmol) and TFA (0.41 mL, 5.30 mmol) to yield 40. Product; details; 38 mg, 54%, collected as a white powder.

Compound #SN 37

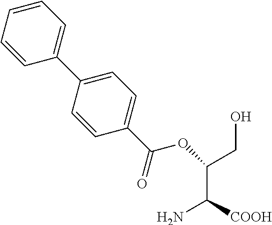 [SMILES: N[C@H](C(=O)O)[C@@H](CO)OC(=O)c1ccc(-c2ccccc2)cc1]

Prepared according to general TFA deprotection procedure step (v). 45 (48 mg, 0.10 mmol), DTT (32 mg, 0.20 mmol) and TFA (0.25 mL, 3.27 mmol) to yield 46. Product details; 25 mg, 57%, collected as a white powder.

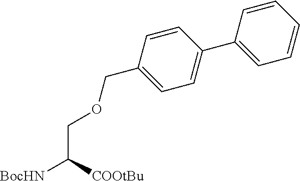

tert-butyl O-([1,1′-biphenyl]-4-ylmethyl)-N-(tert-butoxycarbonyl)-L-serinate, SN 38-a. Prepared according to Williamson ether synthesis. 4 (v) (50 mg, 0.19 mmol) and phenylbenzyl bromide (56 mg, 0.23 mmol) were dissolved dry DMF (2 mL) and cooled to −15° C. using a jacketed flask. NaH (11 mg, 0.27 mmol) was added in portions at −15 0° C. Reaction was then stirred at this for 2 hours. Conversion was very low. Reaction was quenched by addition of 15 mL of water and transferred into separation funnel. Aqueous layer was extracted with 2×15 mL 25% EtOAc in hexanes. 2 mL brine was added to ease separation. Combined organic layers were dried over sodium sulfate and filtered. Filtrate was concentrated in vacuo and residue purified using flash silica gel chromatography 15%-25% EtOAc in hexanes to yield 31 mg, 38% as a colorless oil. (repeated reaction: stirred at room temperature for 2 hours and yield increased to 57%).

Compound #SN 38

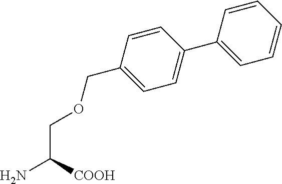 [SMILES: N[C@@H](COCc1ccc(-c2ccccc2)cc1)C(=O)O]

Prepared according to general TFA deprotection procedure scheme 1 step (v). SN 38-a (46 mg, 0.11 mmol), DTT (33 mg, 0.22 mmol) and TFA (0.26 mL, 3.46 mmol). Product details; 30 mg, 71%, collected as a white solid.

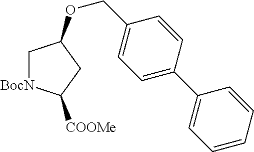 [SMILES: COC(=O)[C@@H]1C[C@H](OCc2ccc(-c3ccccc3)cc2)CN1C(=O)OC(C)(C)C]

1-(tert-butyl) 2-methyl (2S,4S)-4-([1,1′-biphenyl]-4-ylmethoxy)pyrrolidine-1,2-dicarboxylate SN39-a. Prepared according to Williamson ether synthesis. L-Cis-Boc-Hyp-OMe (1.00 g, 4.08 mmol) and phenylbenzyl bromide (1.11 g, 4.49 mmol) were dissolved dry DMF (22.0 mL) and cooled to 0° C. NaH (0.18 g, 4.49 mmol) was added in portions at 0° C. Reaction was then stirred at this for 1 hours. Excess DMF was removed by blowing N₂ over the solution for 2 hours. Residue was taken up in EtOAc and transferred into separating funnel and 20 mL of water added. Aqueous layer was extracted with 3×20 mL 50% EtOAc in hexanes. Combined organic layers were dried over sodium sulfate and filtered. Filtrate was concentrated in vacuo and residue purified using flash silica gel chromatography 5%-30% EtOAc in hexanes to yield 1.30 g, 77% as a colorless oil.

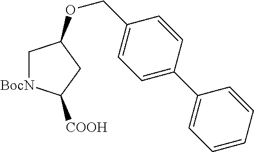 [SMILES: CC(C)(C)OC(=O)N1C[C@@H](OCc2ccc(-c3ccccc3)cc2)C[C@H]1C(=O)O]

(2S,4S)-4-([1,1′-biphenyl]-4-ylmethoxy)-1-(tert-butoxycarbonyl)pyrrolidine-2-carboxylic acid SN39-b. Was synthesized according to general procedure for methyl ester deprotection as shown in scheme 6 step (ii, b). SN39-a (696 mg, 1.69 mmol) was dissolved in THE (2.0 mL) and methanol (1.0 mL) and cooled to 0° C. LiOH·H₂O (355 mg, 8.46 mmol) in 2.0 mL water. Product details; 686 mg, 100%, collected as a white solid.

Compound #SN 39

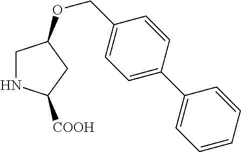 [SMILES: O=C(O)[C@@H]1C[C@H](OCc2ccc(-c3ccccc3)cc2)CN1]

Prepared according to general TFA deprotection procedure scheme 1 step (v). SN39-b (52 mg, 0.15 mmol) in DCM (1.0 mL) and TFA (0.2 mL, 2.40 mmol). Product details; 24 mg, 39%, collected as a white solid.

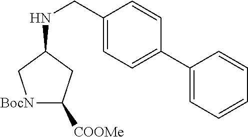 [SMILES: COC(=O)[C@@H]1C[C@H](NCc2ccc(-c3ccccc3)cc2)CN1C(=O)OC(C)(C)C]

1-(tert-butyl) 2-methyl (2S,4S)-4-(([1,1′-biphenyl]-4-ylmethyl)amino)pyrrolidine-1,2-dicarboxylate 11 (a): Prepared according to general procedure for scheme 6 step (i, a). 4 (vi) (150 mg, 0.53 mmol) and biphenyl-4-cabaldehyde (107 mg, 0.58 mmol) and NaBH(OAc)₄ (176 mg, 0.80 mmol). Product details; 200 mg, 92%, collected as a white powder.

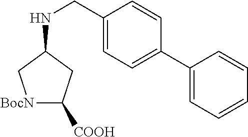 [SMILES: CC(C)(C)OC(=O)N1C[C@@H](NCc2ccc(-c3ccccc3)cc2)C[C@H]1C(=O)O]

(2S,4S)-4-(([1,1′-biphenyl]-4-ylmethyl)amino)-1-(tert-butoxycarbonyl)pyrrolidine-2-carboxylic acid, 12 (a): Prepared according to general procedure for scheme 6 step (ii, a). 11 (b) (100 mg, 0.24 mmol) in THE (3 mL) and MeOH (1 mL) and LiOH·H₂O (61 mg, 1.45 mmol) in water (1 mL). Product details; 82 mg, 84%, collected as a white powder.

Compound #SN40

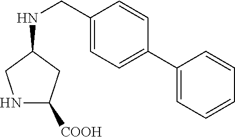 [SMILES: O=C(O)[C@@H]1C[C@H](NCc2ccc(-c3ccccc3)cc2)CN1]

Prepared according to general procedure for scheme 6 step (iii, a). 12 (a) (40 mg, 0.09 mmol) in 1,4-dioxane (1.5 mL) and 4M HCl in 1,4-dioxane (1.2 mL, 5.85 mmol). Product details; 25 mg, 82%, collected as a white powder.

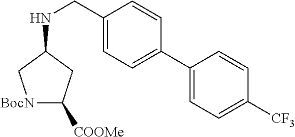 [SMILES: COC(=O)[C@@H]1C[C@H](NCc2ccc(-c3ccc(C(F)(F)F)cc3)cc2)CN1C(=O)OC(C)(C)C]

1-(tert-butyl) 2-methyl (2S,4S)-4-(((4′-(trifluoromethyl)-[1,1′-biphenyl]-4-yl)methyl)amino)pyrrolidine-1,2-dicarboxylate 11 (b): Prepared according to general procedure for scheme 6 step (i, a). 4 (vi) (200 mg, 0.71 mmol) and 4′-Trifluoromethylbiphenyl-4-cabaldehyde (196 mg, 0.78 mmol) and NaBH(OAc)₄ (313 mg, 1.42 mmol). Product details; 245 mg, 72%, collected as a white solid.

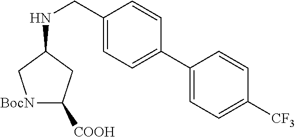 [SMILES: CC(C)(C)OC(=O)N1C[C@@H](NCc2ccc(-c3ccc(C(F)(F)F)cc3)cc2)C[C@H]1C(=O)O]

(2S,4S)-1-(tert-butoxycarbonyl)-4-(((4′-(trifluoromethyl)-[1,1′-biphenyl]-4-yl)methyl)amino)pyrrolidine-2-carboxylic acid, 12 (b): Prepared according to general procedure for scheme 6 step (ii, a). 11 (b) (100 mg, 0.21 mmol) in THE (2 mL) and MeOH (1 mL) and LiOH·H₂O (53 mg, 1.25 mmol) in water (1 mL). Product details; 92 mg, 95%, collected as a white powder.

Compound #SN41

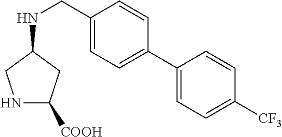 [SMILES: O=C(O)[C@@H]1C[C@H](NCc2ccc(-c3ccc(C(F)(F)F)cc3)cc2)CN1]

Prepared according to general procedure for scheme 6 step (iii, a). 12 (b) (50 mg, 0.10 mmol) in 1,4-dioxane (2.0 mL) and 4M HCl in 1,4-dioxane (1.63 mL, 6.50 mmol). Product details; 33 mg, 69%, collected as a white fine powder.

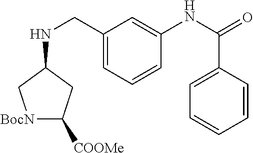 [SMILES: COC(=O)[C@@H]1C[C@H](NCc2cccc(NC(=O)c3ccccc3)c2)CN1C(=O)OC(C)(C)C]

1-(tert-butyl) 2-methyl (2S,4S)-4-((3-benzamidobenzyl)amino)pyrrolidine-1,2-dicarboxylate 11 (c): Prepared according to general procedure for scheme 6 step (i, a). 4 (vi) (100 mg, 0.36 mmol) and 3-(benzylamino)benzaldehyde (80 mg, 0.36 mmol) and NaBH(OAc)₄ (157 mg, 0.71 mmol). Product details; 117 mg, 72%, clear pale-yellow oil.

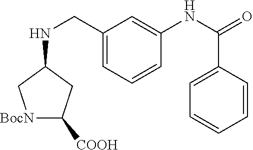 [SMILES: CC(C)(C)OC(=O)N1C[C@@H](NCc2cccc(NC(=O)c3ccccc3)c2)C[C@H]1C(=O)O]

(2S,4S)-4-((3-benzamidobenzyl)amino)-1-(tert-butoxycarbonyl)pyrrolidine-2-carboxylic acid, 12 (c): Prepared according to general procedure for scheme 6 step (ii, a). 11 (c) (117 mg, 0.26 mmol) in THE (3 mL) and MeOH (1.5 mL) and LiOH·H₂O (87 mg, 2.06 mmol) in water (1.5 mL). Product details; 110 mg, 90%, collected as a white powder.

Compound #SN42

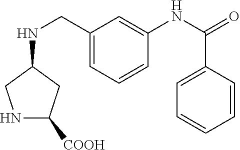 [SMILES: O=C(Nc1cccc(CN[C@@H]2CN[C@H](C(=O)O)C2)c1)c1ccccc1]

Prepared according to general procedure for scheme 6 step (iii, a). 12 (c) (92 mg, 0.19 mmol) in 1,4-dioxane (4.0 mL) and 4M HCl in 1,4-dioxane (3.5 mL, 12.00 mmol). Product details; 83 mg, 96%, collected as a white fine powder.

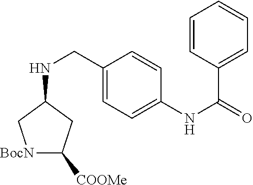 [SMILES: COC(=O)[C@@H]1C[C@H](NCc2ccc(NC(=O)c3ccccc3)cc2)CN1C(=O)OC(C)(C)C]

1-(tert-butyl) 2-methyl (2S,4S)-4-((4-benzamidobenzyl)amino)pyrrolidine-1,2-dicarboxylate 11 (d): Prepared according to general procedure for scheme 6 step (i, a). 4 (vi) (100 mg, 0.36 mmol) and N-(4-formylphenyl)benzamide (80 mg, 0.36 mmol) and NaBH(OAc)₄ (157 mg, 0.71 mmol). Product details; 109 mg, 68%, clear pale-yellow oil

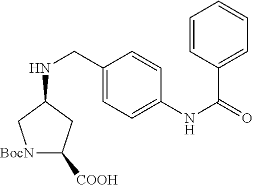 [SMILES: CC(C)(C)OC(=O)N1C[C@@H](NCc2ccc(NC(=O)c3ccccc3)cc2)C[C@H]1C(=O)O]

(2S,4S)-4-((4-benzamidobenzyl)amino)-1-(tert-butoxycarbonyl)pyrrolidine-2-carboxylic acid, 12 (d): Prepared according to general procedure for scheme 6 step (ii, a). 11 (d) (109 mg, 0.24 mmol) in THF (3 mL) and MeOH (1.5 mL) and LiOH·H₂O (87 mg, 2.06 mmol) in water (1.5 mL). Product details; 101 mg, 89%, collected as a white solid.

Compound #SN43

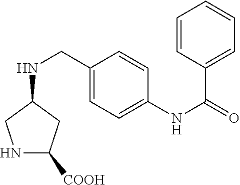 [SMILES: O=C(Nc1ccc(CN[C@@H]2CN[C@H](C(=O)O)C2)cc1)c1ccccc1]

Prepared according to general procedure for scheme 6 step (iii, a). 12 (d) (88 mg, 0.18 mmol) in 1,4-dioxane (3.0 mL) and 4M HCl in 1,4-dioxane (3.5 mL, 12.00 mmol). Product details; 69 mg, 91%, collected as a light-brown powder.

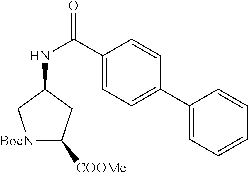 [SMILES: COC(=O)[C@@H]1C[C@H](NC(=O)c2ccc(-c3ccccc3)cc2)CN1C(=O)OC(C)(C)C]

1-(tert-butyl) 2-methyl (2S,4S)-4-([1,1′-biphenyl]-4-carboxamido)pyrrolidine-1,2-dicarboxylate, 23 (a): Prepared according to general procedure for scheme 7 step (a, ii). 4 (vi) (100 mg, 0.36 mmol)), TEA (0.15 mL, 1.07 mmol), DMAP (4.4 mg, 0.04 mmol) dissolved in DCM (3.0 mL) and biphenyl-4-carbonyl chloride (77 mg, 0.37 mmol). Product details; 91 mg, 60%, as a white spongy powder.

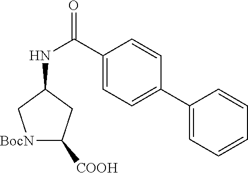 [SMILES: CC(C)(C)OC(=O)N1C[C@@H](NC(=O)c2ccc(-c3ccccc3)cc2)C[C@H]1C(=O)O]

(2S,4S)-4-([1,1′-biphenyl]-4-carboxamido)-1-(tert-butoxycarbonyl)pyrrolidine-2-carboxylic acid, 24 (a): Prepared according to general procedure for scheme 7 step (b,ii). 23 (a) (90 mg, 0.21 mmol) in THE (3 mL) and MeOH (1.0 mL) and LiOH·H₂O (53 mg, 1.27 mmol) in water (1.0 mL). Product details; 87 mg, 100%, collected as a white powder.

Compound #SN45

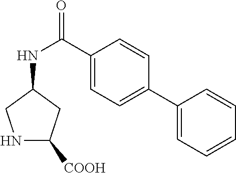 [SMILES: O=C(N[C@@H]1CN[C@H](C(=O)O)C1)c1ccc(-c2ccccc2)cc1]

Prepared according to general procedure for scheme 7 step (c, ii). 24 (a) (40 mg, 0.10 mmol) in 1,4-dioxane (2.0 mL) and 4M HCl in 1,4-dioxane (1.6 mL, 6.33.00 mmol). Product details; 40 mg, 100%, white powder.

Compound #SN46

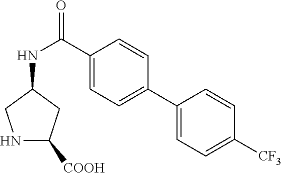 [SMILES: O=C(N[C@@H]1CN[C@H](C(=O)O)C1)c1ccc(-c2ccc(C(F)(F)F)cc2)cc1]

This compound is prepared according to the general procedure for scheme 7 step (c, ii). 24 (a) (40 mg, 0.10 mmol) in 1,4-dioxane (2.0 mL) and 4M HCl in 1,4-dioxane (1.6 mL, 6.33.00 mmol). Product details; 40 mg, 100%, white powder.

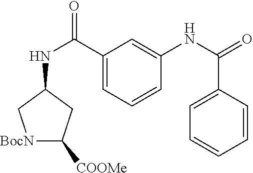 [SMILES: COC(=O)[C@@H]1C[C@H](NC(=O)c2cccc(NC(=O)c3ccccc3)c2)CN1C(=O)OC(C)(C)C]

1-(tert-butyl) 2-methyl (2S,4S)-4-(3-benzamidobenzamido)pyrrolidine-1,2-dicarboxylate, 23 (b): Prepared according to general procedure for scheme 7 step (a, ii). 4 (vi) (200 mg, 0.71 mmol)), TEA (0.35 mL, 2.28 mmol), DMAP (17 mg, 0.14 mmol) dissolved in DCM (5.0 mL) and 3-benzamidobenzoyl chloride (278 mg, 1.07 mmol). Product details; 215 mg, 65%, as a foamy white powder.

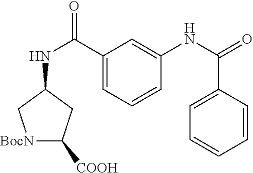 [SMILES: CC(C)(C)OC(=O)N1C[C@@H](NC(=O)c2cccc(NC(=O)c3ccccc3)c2)C[C@H]1C(=O)O]

(2S,4S)-4-(3-benzamidobenzamido)-1-(tert-butoxycarbonyl)pyrrolidine-2-carboxylic acid, 24 (b): Prepared according to general procedure for scheme 7 step (b,ii). 23 (b) (197 mg, 0.42 mmol) in THE (4 mL) and MeOH (2.0 mL) and LiOH·H₂O (141 mg, 3.37 mmol) in water (2.0 mL). Product details; 193 mg, 100%, white solid.

Compound #SN47

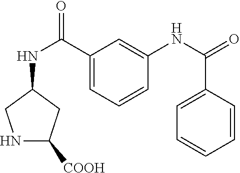 [SMILES: O=C(Nc1cccc(C(=O)N[C@@H]2CN[C@H](C(=O)O)C2)c1)c1ccccc1]

Prepared according to general procedure for scheme 7 step (c, ii). 24 (b) (177 mg, 0.39 mmol) in 1,4-dioxane (4.0 mL) and 4M HCl in 1,4-dioxane (4 mL, 16.00 mmol). Product details; 166 mg, 100%, white powder.

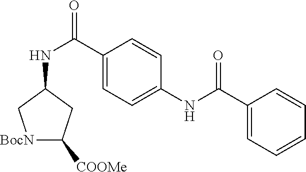 [SMILES: COC(=O)[C@@H]1C[C@H](NC(=O)c2ccc(NC(=O)c3ccccc3)cc2)CN1C(=O)OC(C)(C)C]

1-(tert-butyl) 2-methyl (2S,4S)-4-(4-benzamidobenzamido)pyrrolidine-1,2-dicarboxylate, 23 (c): Prepared according to general procedure for scheme 7 step (a, ii). 4 (vi) (200 mg, 0.71 mmol)), TEA (0.35 mL, 2.28 mmol), DMAP (17 mg, 0.14 mmol) dissolved in DCM (5.0 mL) and 3-benzamidobenzoyl chloride (278 mg, 1.07 mmol). Product details; 143 mg, 43%, as a foamy white powder.

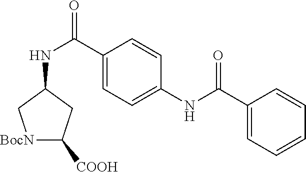 [SMILES: CC(C)(C)OC(=O)N1C[C@@H](NC(=O)c2ccc(NC(=O)c3ccccc3)cc2)C[C@H]1C(=O)O]

(2S,4S)-4-(4-benzamidobenzamido)-1-(tert-butoxycarbonyl)pyrrolidine-2-carboxylic acid, 24 (b): Prepared according to general procedure for scheme 7 step (b,ii). 23 (c) (124 mg, 0.27 mmol) in THE (3 mL) and MeOH (1.5 mL) and LiOH·H₂O (89 mg, 2.12 mmol) in water (1.0 mL). Product details; 106 mg, 88%, white solid.

Compound #SN48

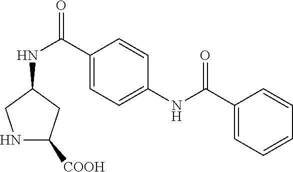 [SMILES: O=C(Nc1ccc(C(=O)N[C@@H]2CN[C@H](C(=O)O)C2)cc1)c1ccccc1]

Prepared according to general procedure for scheme 7 step (c, ii). 24 (c) (177 mg, 0.39 mmol) in 1,4-dioxane (4.0 mL) and 4M HCl in 1,4-dioxane (4 mL, 16.00 mmol). Product details; 166 mg, 100%, white powder.

Compound #SN50

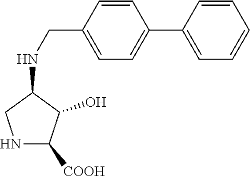 [SMILES: O=C(O)[C@H]1NC[C@@H](NCc2ccc(-c3ccccc3)cc2)[C@@H]1O]

Prepared according to general procedure for scheme 6 step (iii, a). 61 (36 mg, 0.07 mmol) and 4M HCl in 1,4-dioxane (6.0 mL, 24.00 mmol). Product details; 37 mg, 100%, white powder.

### Dihydroxyproline Ester Derivatives

Synthesis: Experimental Procedures

Acyl chlorides used were synthesized according to scheme 8.

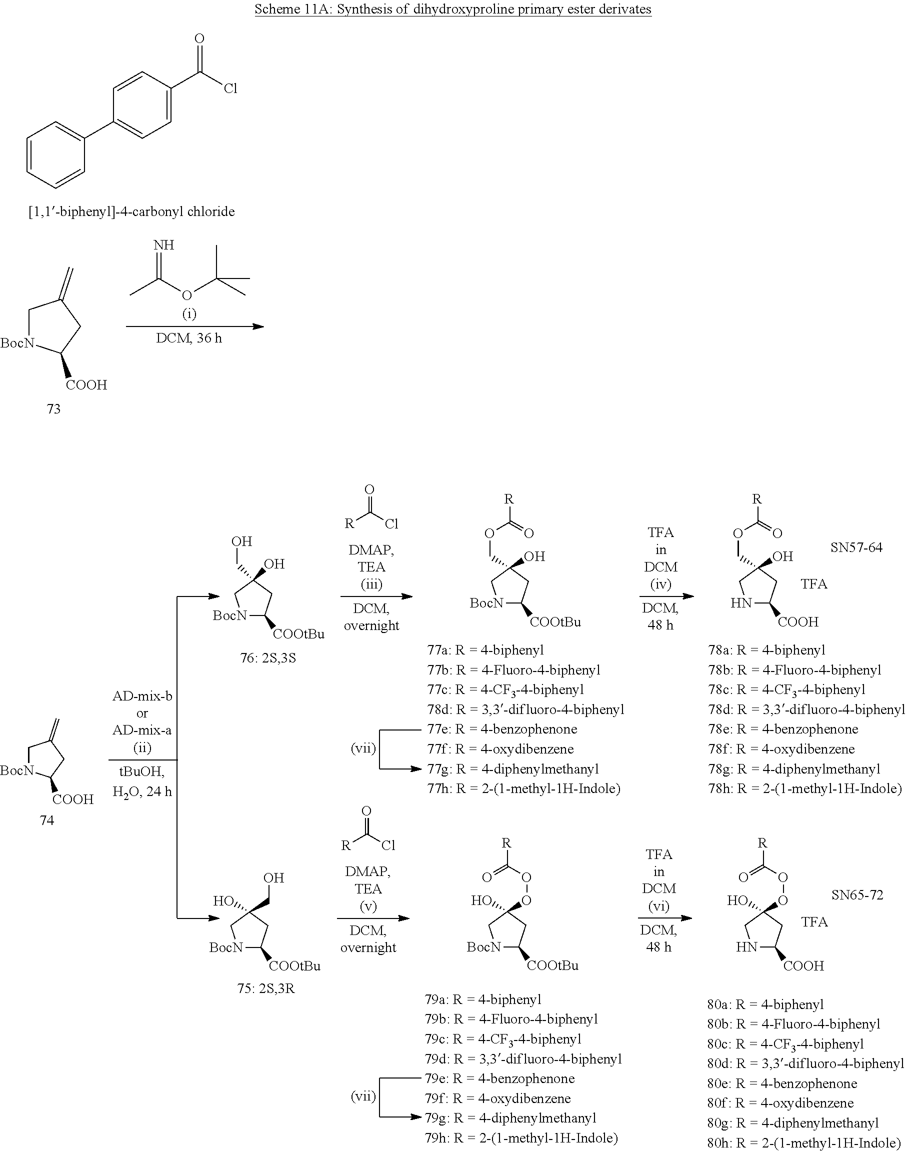

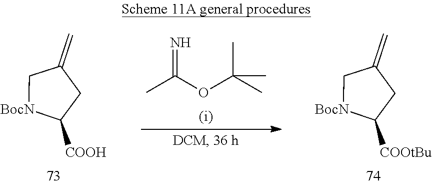

di-tert-butyl (S)-4-methylenepyrrolidine-1,2-dicarboxylate, 74. (S)-1-(tert-butoxycarbonyl)-4-methylenepyrrolidine-2-carboxylic acid, 73 (4.50 g, 19.80 mmol) was dissolved in dry DCM (30.0 mL) and cooled to 0° C. 11.0 mL of tert-Butyl 2,2,2-trichloroacetimidate (12.98 g, 59.40 mmol) was added dropwise under N₂ and the mixture was stirred for 36 hours at room temperature. Precipitates were filtered out and filtrate concentrated in vacuo. The residue was purified using flash silica gel chromatography (0-15% ethyl acetate in hexanes) to obtain a pure colorless oil. Product details; 5.05 g, 90% yield as a colorless oil.

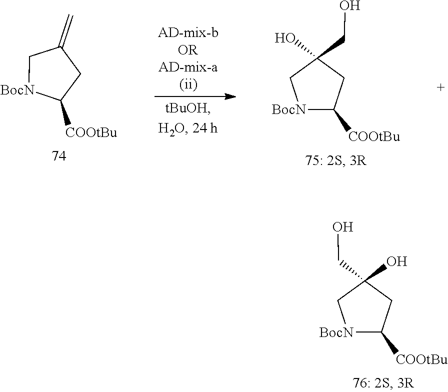

di-tert-butyl (2S,4R)-4-hydroxy-4-(hydroxymethyl)pyrrolidine-1,2-dicarboxylate, 75 and di-tert-butyl (2 S, 4S)-4-hydroxy-4-(hydroxymethyl)pyrrolidine-1,2-dicarboxylate, 76 were prepared as shown below. For AD-mix-α example;

AD-mi-α (14.30 g) was dissolved in t-BuOH (15.0 mL) and water (20.0 mL) at room temperature until all contents dissolved. The solution was then cooled to 0° C. and 74, (2.90 g, 10.21 mmol) in t-BuOH (5 mL) added. The mixture was stirred at 0° C. for 36 hours until complete conversion. The reaction was quenched by the addition of Na₂SO₃ (15.31 g, 1.5 g/mmol of alkene) at 0° C. The mixture was let to stir at room temperature for 1 hour then transferred into separating funnel and diluted with EtOAc (30.0 mL) and brine (20.0 mL). Aqueous layer was extracted with EtOAc (3×30.0 mL). Combined EtOAc layers were then dried over Na₂SO₄ and filtered off. The filtrate was concentrated in vacuo and product purified using flash silica gel chromatography (15%-100% EtOAc in hexanes with 2% TEA). Total yield; (2S, 3R) 75, plus (2S, 3S) 76, 9.53 g, 93% as a white solid (75) or colorless oil (76) that turns solid on freezing. AD-mix-β follows same protocol as AD-Mix-α described above.

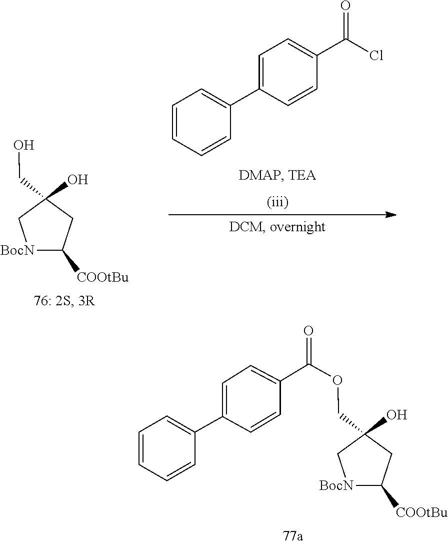

### General Procedure for Scheme 11A Step (iii)

76 (92 mg, 0.29 mmol, 1.0 eqv), DMAP (7 mg, 0.06 mmol, 0.2 eqv), biphenyl]-4-carbonyl chloride (82 mg, 0.38 mmol, 1.3 eqv) were dissolved in dry DCM (7 mL) then cooled to 0° C. TEA (81 μL, 0.58 mmol, 2.0 eqv) was added dropwise under N₂ gas and the reaction mixture left to warm up to room temperature and stirred overnight. The reaction was quenched with saturated NH₄Cl (5 mL) and DCM evaporated. 20 mL of EtOAc was added and the Organic layer washed with saturated NH₄Cl (2×5 mL), saturated NaHCO₃ (3×10 mL) and brine/water mixture 1:1 (1×15 mL) then dried over Na₂SO₄ and filtered off. The filtrate was concentrated in vacuo and the product purified using flash silica gel chromatography (10-40% ethyl acetate in hexanes with 2% TEA). Product details; 123 mg, 85% yield as a white solid

### General Procedure for Scheme 11A Step (iv)

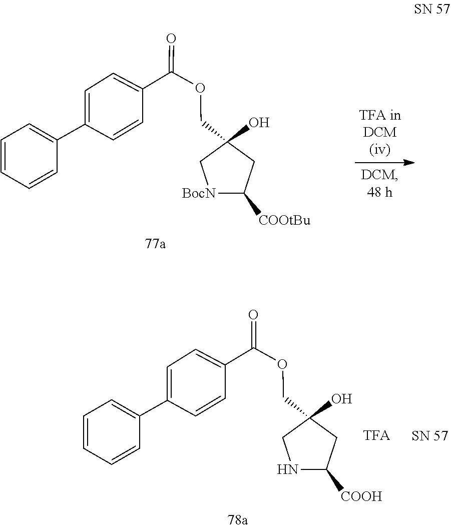

77a (SN 57) (87 mg, 0.17 mmol) was dissolved in dry DCM (2 mL) and cooled to 0° C. Trifluoroacetic acid (TFA) (2 mL, 26.14 mmol, 100-154 eqv) was added dropwise under N₂ at 0° C. and the reaction mixture let to warm up to room temperature and stirred for 48 hours until 100% conversion. TFA was completely removed under reduced pressure using DCM to form a residue was suspended in chilled ether and filtered off. In case of oily residue, the product was purified by trituration in methanol and chilled diethyl ether and filtered off. The precipitated was washed with chilled ether. Product details; 77 mg, 99.5% as a white powder.

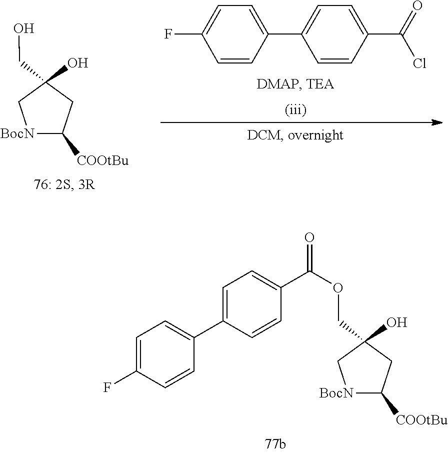

77b was synthesized according to the general procedure for scheme 11A step (iii). 76 (66 mg, 0.21 mmol), DMAP (5.1 mg, 0.04 mmol), 4-fluorobiphenyl]-4-carbonyl chloride (63 mg, 0.27 mmol) dissolved in dry DCM (8 mL). TEA used (˜0.1 mL, 0.42 mmol). Product details; 32 mg, 30% as a colorless oil.

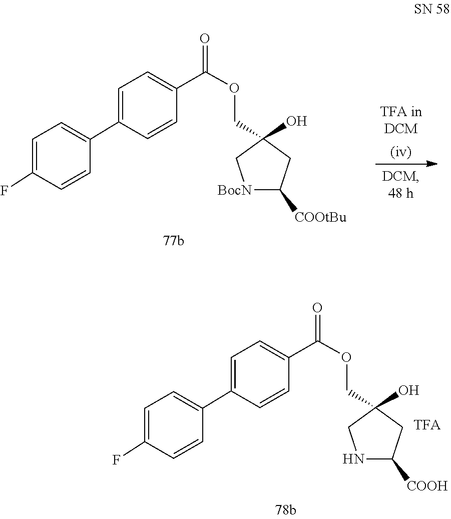

78b (SN 58) was synthesized according to the general procedure for scheme 11A step (iv). 77b (37 mg, 0.07 mmol) was dissolved in dry DCM (2.5 mL) and cooled to 0° C. Trifluoroacetic acid (TFA) (2.0 mL, 26.14 mmol). Product details; 20 mg, 59% light pink powder.

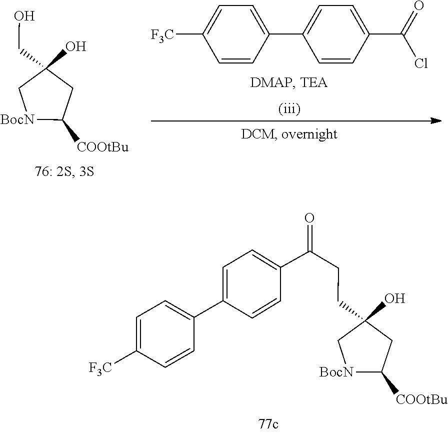

77c was synthesized according to the general procedure for scheme 11A step (iii).

76 (80 mg, 0.26 mmol), DMAP (6.3 mg, 0.05 mmol), 4-trifluoromethylbiphenyl]-4-carbonyl chloride (96 mg, 0.34 mmol) dissolved in dry DCM (8 mL). TEA used (˜0.1 mL, 0.52 mmol). Product details; 109 mg, 74% as a colorless oil which solidifies to a white solid on cooling.

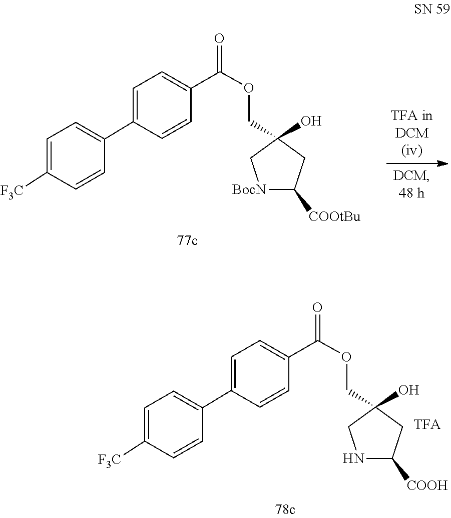

78c (SN 59) was synthesized according to the general procedure for scheme 11A step (iv). 77c (37 mg, 0.07 mmol) was dissolved in dry DCM (2.5 mL) and cooled to 0° C. Trifluoroacetic acid (TFA) (2.0 mL, 26.14 mmol). Product details; 20 mg, 59% light pink powder.

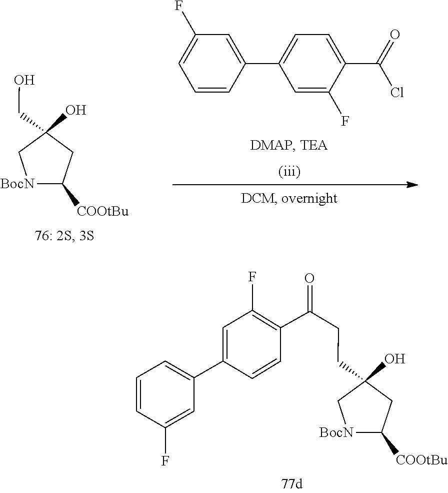

77d was synthesized according to the general procedure for scheme 11A step (iii). 76 (77 mg, 0.24 mmol), DMAP (6.0 mg, 0.05 mmol), 3,3′-difluoro-[1,1′-biphenyl]-4-carbonyl chloride (80 mg, 0.32 mmol) dissolved in dry DCM (8 mL). TEA used (˜0.1 mL, 0.50 mmol). Product details; 103 mg, 79% colorless oil.

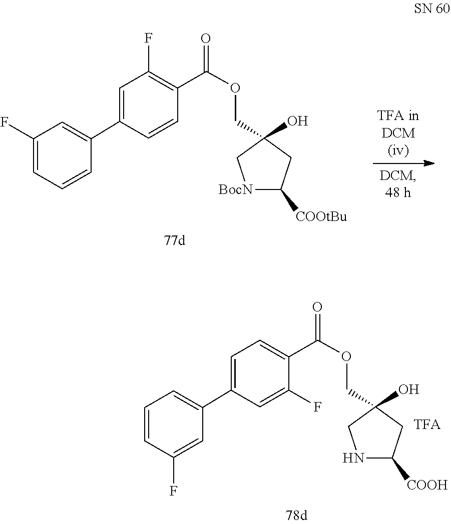

78d (SN 60) was synthesized according to the general procedure for scheme 11A step (iv). 77d (103 mg, 0.19 mmol) was dissolved in dry DCM (1.5 mL) and cooled to 0° C. Trifluoroacetic acid (TFA) (1.5 mL, 19.60 mmol). Product details; 75 mg, 79% white powder.

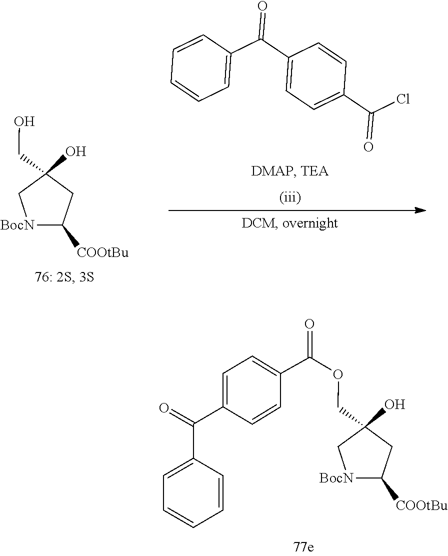

77e was synthesized according to the general procedure for scheme 11A step (iii). 76 (137 mg, 0.43 mmol), DMAP (11 mg, 0.09 mmol), 4-benzoylbenzoyl chloride (137 mg, 0.56 mmol) dissolved in dry DCM (8 mL). TEA used (˜0.15 mL, 0.86 mmol). Product details; 175 mg, 77% colorless oil.

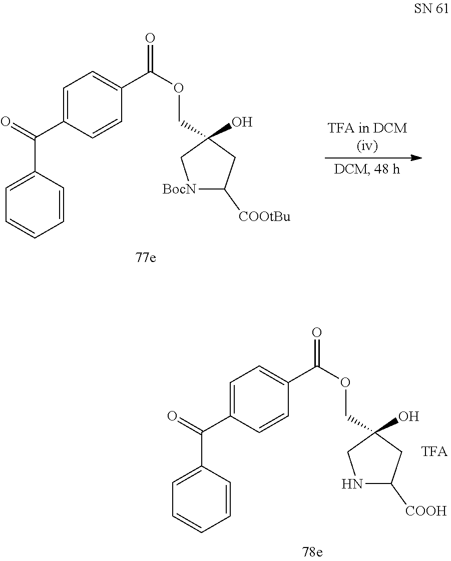

78e (SN 61) was synthesized according to the general procedure for scheme 11A step (iv). 77e (83 mg, 0.16 mmol) was dissolved in dry DCM (1.5 mL) and cooled to 0° C. Trifluoroacetic acid (TFA) (1.5 mL, 19.60 mmol). Product details; 55 mg, 72% light pink powder.

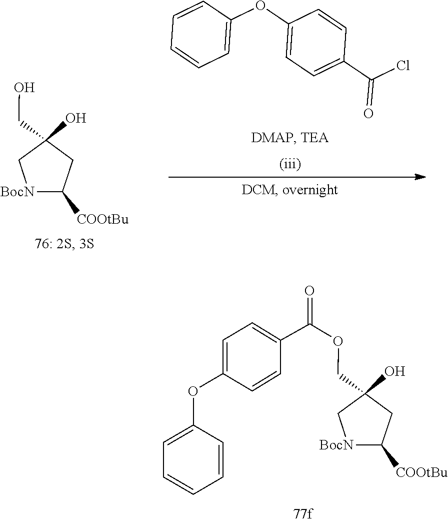

77f was synthesized according to the general procedure for scheme 11A step (iii). 76 (80 mg, 0.25 mmol), DMAP (6 mg, 0.05 mmol), 4-phenoxybenzoyl chloride (76 mg, 0.33 mmol) dissolved in dry DCM (4 mL). TEA used (˜0.10 mL, 0.50 mmol). Product details; 92 mg, 71% colorless oil.

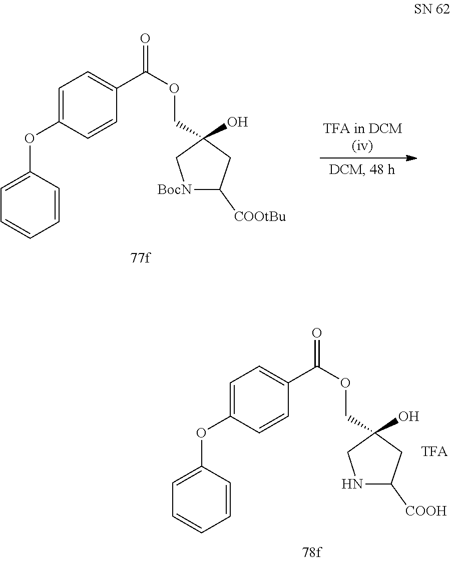

78f (SN 62) was synthesized according to the general procedure for scheme 11A step (iv). 77f (43 mg, 0.08 mmol) was dissolved in dry DCM (3 mL) and cooled to 0° C. Trifluoroacetic acid (TFA) (1.5 mL, 19.60 mmol). Product details; 22 mg, 54% white solid.

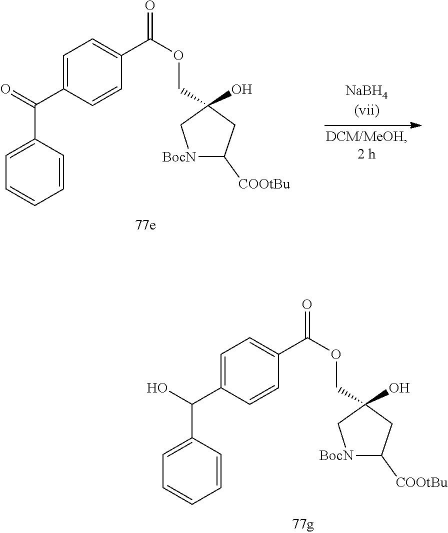

77g was synthesized as follows; 77e (100 mg, 0.19 mmol) was dissolved in DCM (1 mL)/methanol 2.0 mL mixture and cooled to 0° C. NaBH4 (14.4 mg, 0.38 mmol) was added in one portion. The reaction was let to warm up to room temperature and stirred for 2 hours until complete conversion as monitored by TLC. Reaction was quenched by addition of acetone and transferred into separating funnel and 5.0 mL of water added then product extracted with DCM or EtOAc (10 mL×3). Combined organic layers were washed with brine and water 1:1 (2×10 mL) and then dried over Na₂SO₄ and filtered off. Filtrate was concentrated in vacuo and residue purified using silica gel flash chromatography (EtOAc in hexanes 15%-80% with 2% TEA). Product details; 71 mg, 71% spongy white solid.

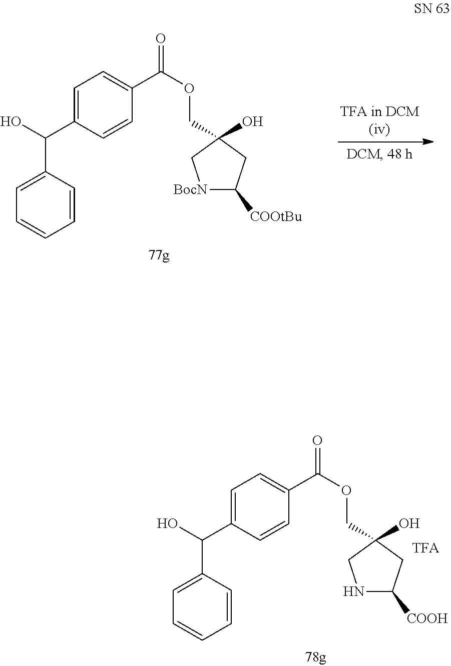

78g (SN 63) was synthesized according to the general procedure for scheme 11A step (iv). 77g (70 mg, 0.13 mmol) was dissolved in dry DCM (1.5 mL) and cooled to 0° C. Trifluoroacetic acid (TFA) (1 mL, 13.07 mmol). Product details; 68 mg, 100% off-white solid.

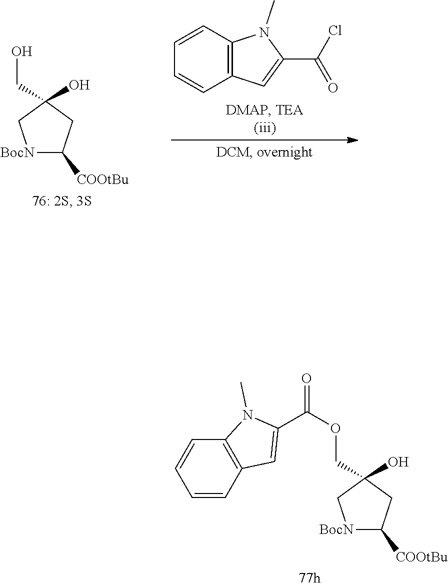

77h was synthesized according to the general procedure for scheme 11A step (iii). 76 (78 mg, 0.24 mmol), DMAP (6 mg, 0.05 mmol), 1-methyl-1H-indole-2-carbonyl chloride (60 mg, 0.31 mmol) dissolved in dry DCM (5 mL). TEA used (˜0.10 mL, 0.50 mmol). Product details; 93 mg, 80% white powder.

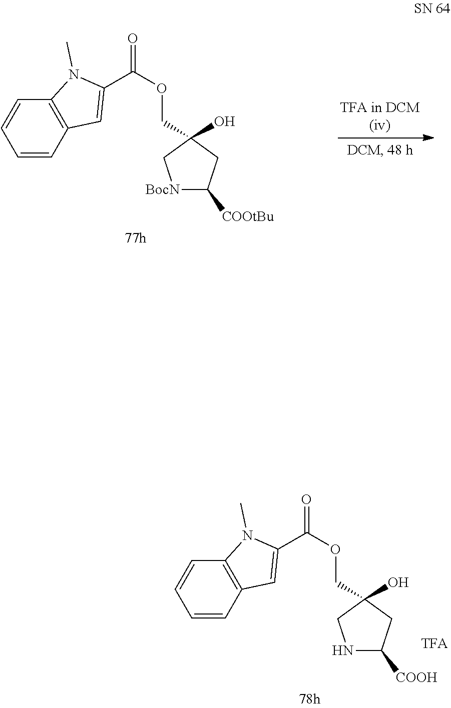

78h (SN 64) was synthesized according to the general procedure for scheme 11A step (iv). 77h (93 mg, 0.20 mmol) was dissolved in dry DCM (2.5 mL) and cooled to 0° C. Trifluoroacetic acid (TFA) (2.0 mL, 26.14 mmol). Product details; 63 mg, 74% light pink powder.

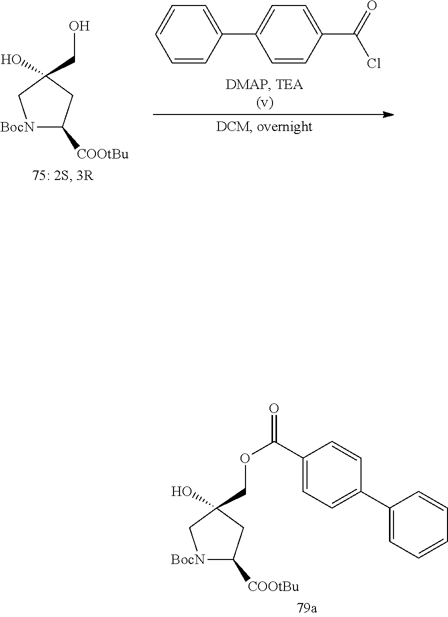

79a was synthesized according to the general procedure for scheme 11A step (iii). 75 (100 mg, 0.32 mmol), DMAP (7.6 mg, 0.06 mmol), biphenyl-4-carbonyl chloride (89 mg, 0.41 mmol) dissolved in dry DCM (10 mL). TEA used (˜0.1 mL, 0.63 mmol). Product details; 156 mg, 99% colorless oil.

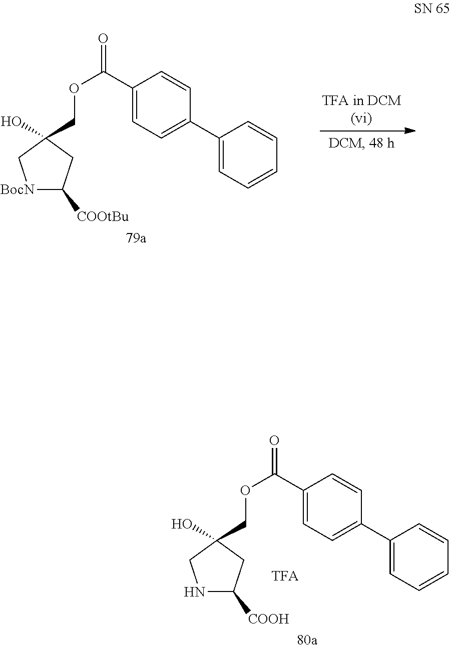

80a (SN 65) was synthesized according to the general procedure for scheme 11A step (iv). 79a (100 mg, 0.20 mmol) was dissolved in dry DCM (3 mL) and cooled to 0° C. Trifluoroacetic acid (TFA) (2.0 mL, 26.14 mmol). Product details; 84 mg, 92% light orange powder.

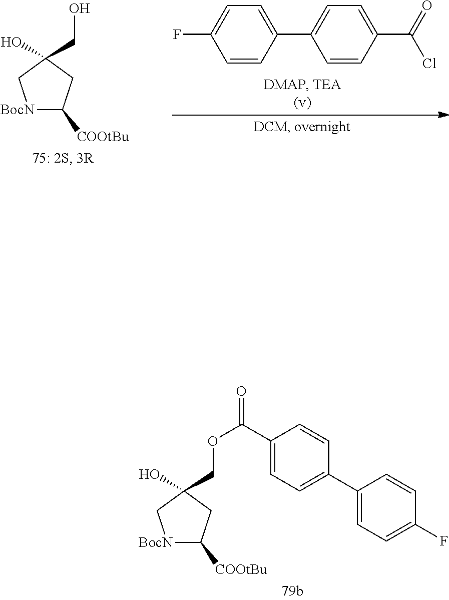

79b was synthesized according to the general procedure for scheme 11A step (iii). 75 (80 mg, 0.25 mmol), DMAP (6.2 mg, 0.05 mmol), 4-fluorobiphenyl]-4-carbonyl chloride (77 mg, 0.33 mmol) dissolved in dry DCM (3 mL). TEA used (˜0.1 mL, 0.50 mmol). Product details; 84 mg, 65% as a colorless oil.

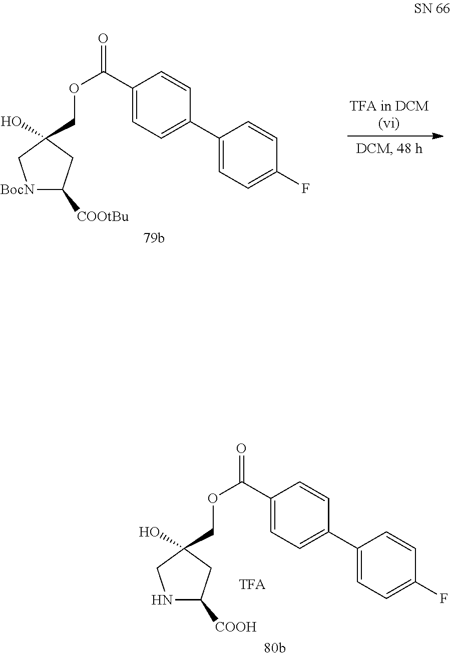

80b (SN 66) was synthesized according to the general procedure for scheme 11A step (iv). 79b (84 mg, 0.16 mmol) was dissolved in dry DCM (2.5 mL) and cooled to 0° C. Trifluoroacetic acid (TFA) (1.5 mL, 19.60 mmol). Product details; 45 mg, 58% off-white powder.

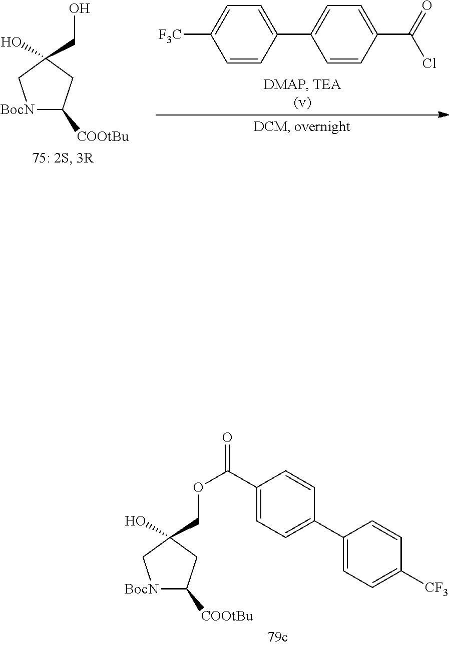

79c was synthesized according to the general procedure for scheme 11A step (iii). 75 (80 mg, 0.25 mmol), DMAP (6.2 mg, 0.05 mmol), 4-trifluoromethylbiphenyl]-4-carbonyl chloride (93 mg, 0.33 mmol) dissolved in dry DCM (3 mL). TEA used (˜0.1 mL, 0.50 mmol). Product details; 70 mg, 50% as a colorless oil.

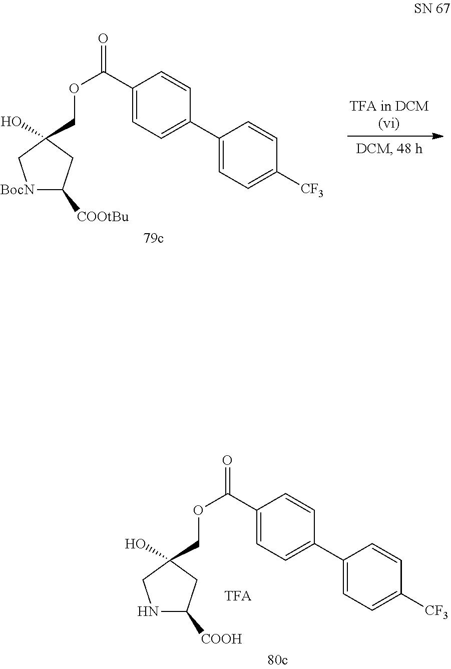

80c (SN 67) was synthesized according to the general procedure for scheme 11A step (iv). 79c (70 mg, 0.12 mmol) was dissolved in dry DCM (2.5 mL) and cooled to 0° C. Trifluoroacetic acid (TFA) (1.5 mL, 19.60 mmol). Product details; 59 mg, 91% light purple powder.

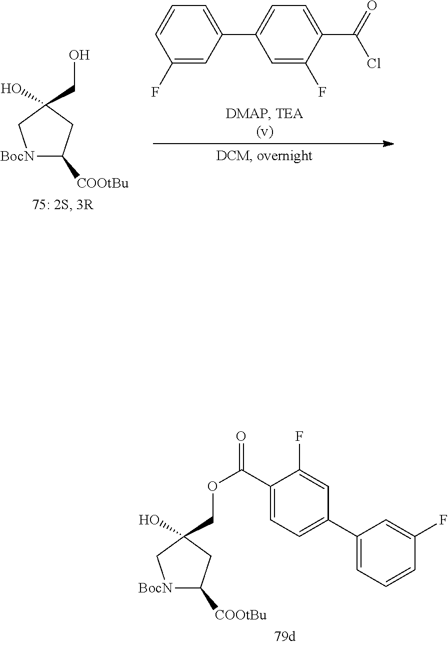

79d was synthesized according to the general procedure for scheme 11A step (iii). 75 (80 mg, 0.25 mmol), DMAP (6.0 mg, 0.05 mmol), 3,3′-difluoro-[1,1′-biphenyl]-4-carbonyl chloride (96 mg, 0.38 mmol) dissolved in dry DCM (3 mL). TEA used (˜0.1 mL, 0.50 mmol). Product details; 115 mg, 86% as a colorless oil.

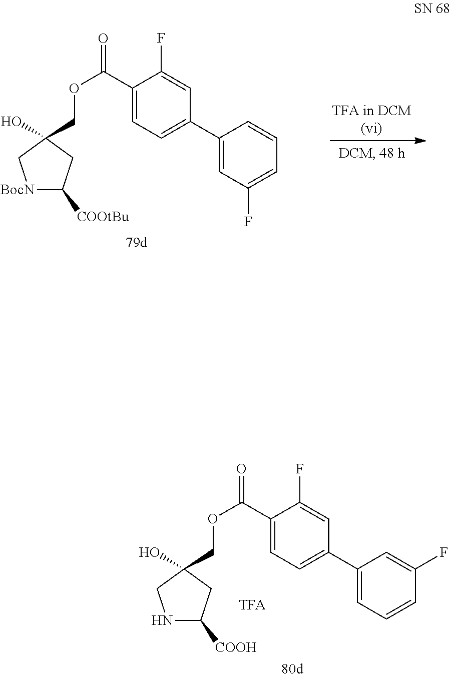

80d (SN 68) was synthesized according to the general procedure for scheme 11A step (iv). 79d (115 mg, 0.22 mmol) was dissolved in dry DCM (2.5 mL) and cooled to 0° C. Trifluoroacetic acid (TFA) (1.5 mL, 19.60 mmol). Product details; 100 mg, 94% off-white powder.

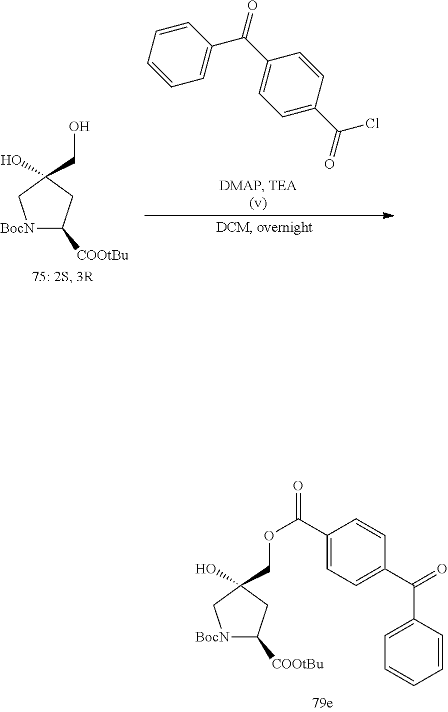

79e was synthesized according to the general procedure for scheme 11A step (iii). 75 (100 mg, 0.32 mmol), DMAP (8.0 mg, 0.06 mmol), 4-benzoylbenzoyl chloride (116 mg, 0.47 mmol) dissolved in dry DCM (3 mL). TEA used (˜0.1 mL, 0.63 mmol). Product details; 135 mg, 82% white solid.

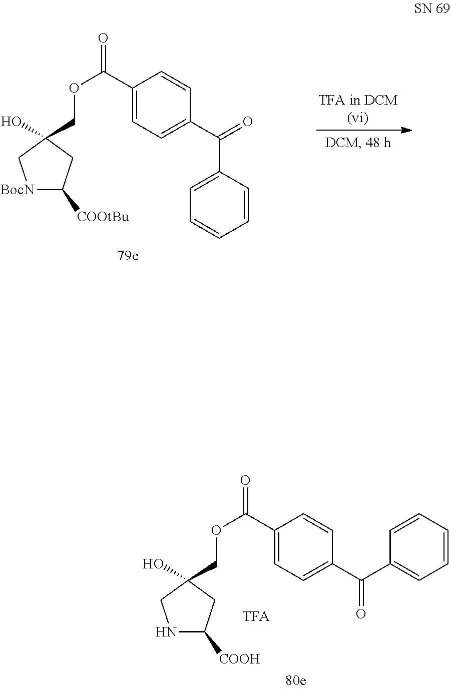

80e (SN 69) was synthesized according to the general procedure for scheme 11A step (iv). 79e (76 mg, 0.15 mmol) was dissolved in dry DCM (2.5 mL) and cooled to 0° C. Trifluoroacetic acid (TFA) (1.5 mL, 19.60 mmol). Product details; 66 mg, 94% light pink powder.

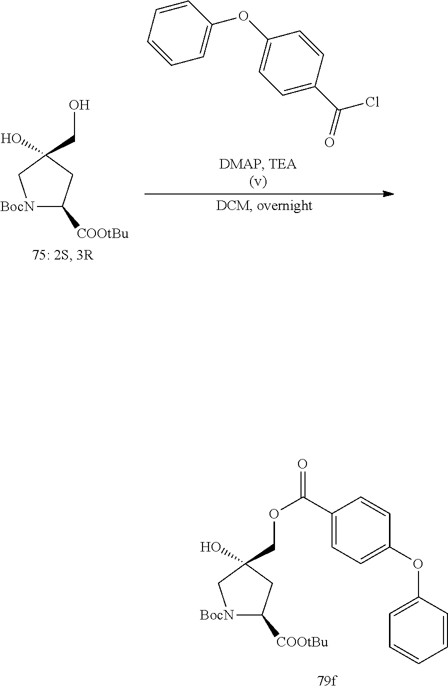

79f was synthesized according to the general procedure for scheme 11A step (iii). 75 (80 mg, 0.25 mmol), DMAP (6 mg, 0.05 mmol), 4-phenoxybenzoyl chloride (76 mg, 0.33 mmol) dissolved in dry DCM (4 mL). TEA used (˜0.1 mL, 0.50 mmol). Product details; 104 mg, 80% white solid.

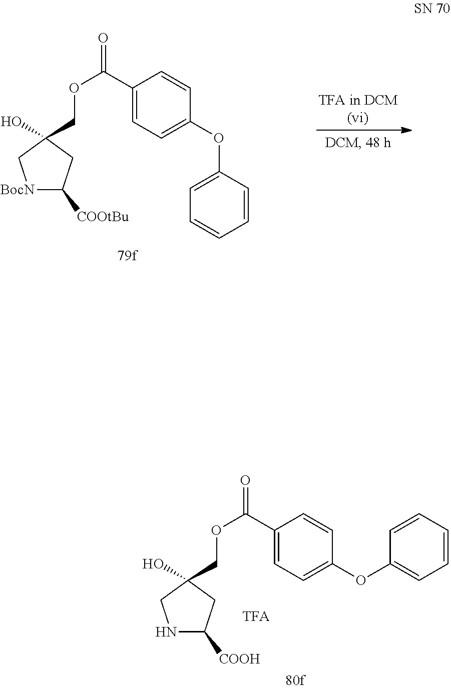

80f (SN 70) was synthesized according to the general procedure for scheme 11A step (iv). 79f (104 mg, 0.20 mmol) was dissolved in dry DCM (3 mL) and cooled to 0° C. Trifluoroacetic acid (TFA) (2.0 mL, 26.14 mmol). Product details; 72 mg, 76% off-white powder.

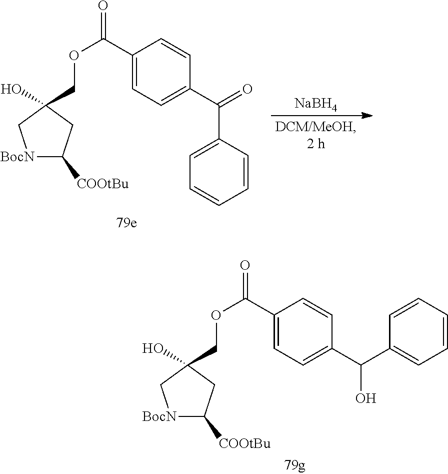

79g was synthesized as compound 77g; 79e (59 mg, 0.11 mmol) was dissolved in DCM (1 mL)/methanol 2.0 mL mixture and cooled to 0° C. NaBH4 used (8.5 mg, 0.23 mmol). Product details; 45 mg, 76% colorless oil.

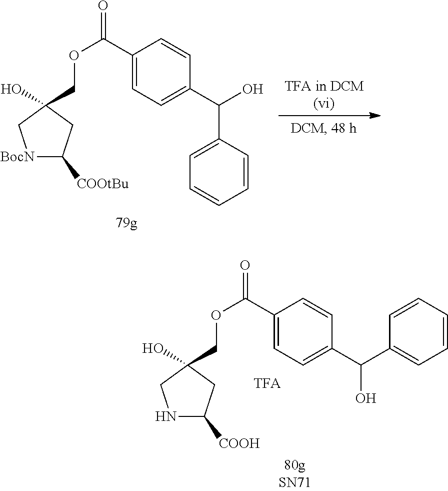

80g (SN 71) was synthesized according to the general procedure for scheme 11A step (iv). 79g (31 mg, 0.06 mmol) was dissolved in dry DCM (1.5 mL) and cooled to 0° C. Trifluoroacetic acid (TFA) (1.5 mL, 13.07 mmol). Product details; 25 mg, 86% off-white powder

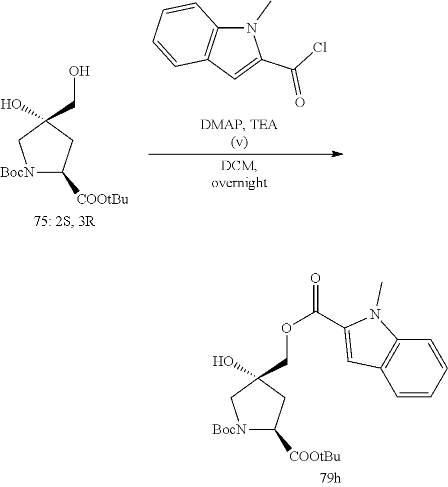

79h was synthesized according to the general procedure for scheme 11A step (iii). 75 (78 mg, 0.24 mmol), DMAP (6 mg, 0.05 mmol), 1-methyl-1H-indole-2-carbonyl chloride (60 mg, 0.31 mmol) dissolved in dry DCM (5 mL). TEA used (˜0.10 mL, 0.50 mmol). Product details; 63 mg, 54% white powder.

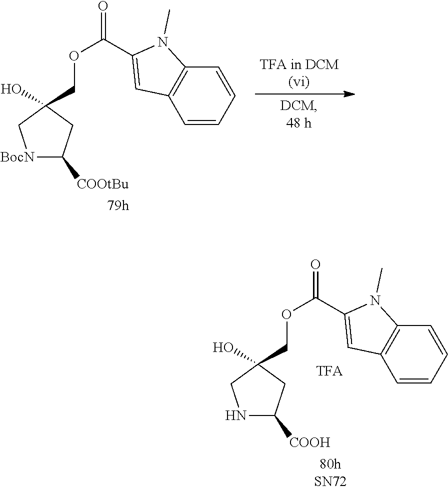

80h (SN 72) was synthesized according to the general procedure for scheme 11A step (iv). 79h (63 mg, 0.13 mmol) was dissolved in dry DCM (2.5 mL) and cooled to 0° C. Trifluoroacetic acid (TFA) (2.0 mL, 26.14 mmol). Product details; 40 mg, 70% off-white powder.

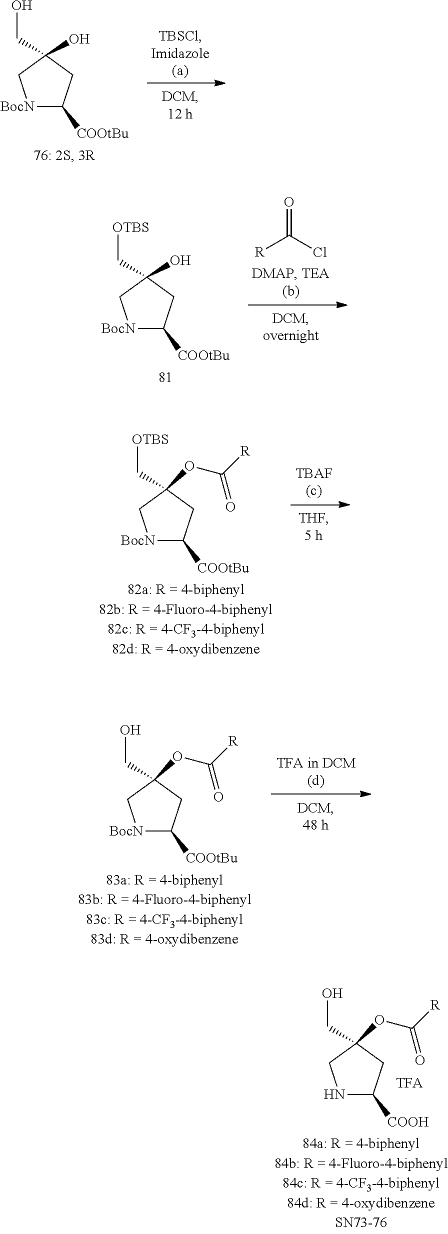

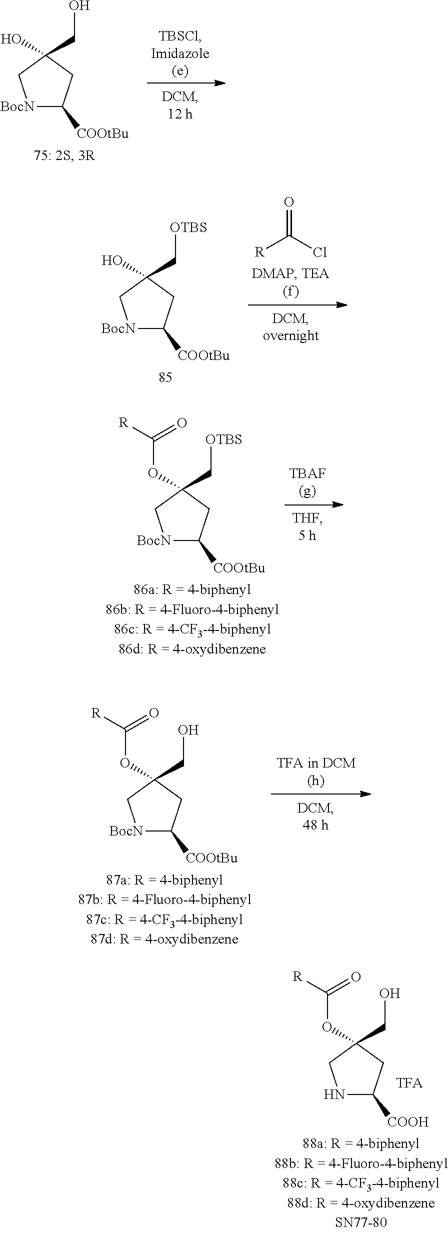

### Scheme 11B General Procedures

### General Synthesis Procedures for Repeated Steps

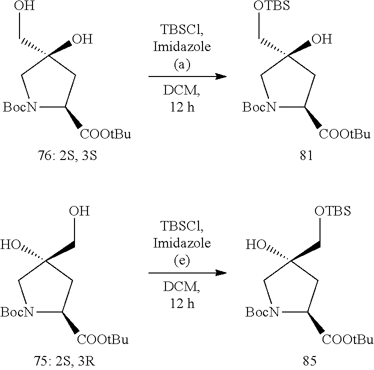

TBS installation (scheme 11B step (a) and step (e): 81 and 85 were prepared as follows; 76 (311 mg, 0.98 mmol, 1.0 eqv) or 75 (404 mg, 1.27 mmol, 1.0 eq) and imidazole (1.5 eq) were dissolved in dry DCM (7 mL) and catalytic amount of DMF (0.1 mL) then cooled to 0° C. TBSCI (1.1 eq) was added dropwise under inert conditions. The mixture was stirred for 24 hours at room temperature and quenched with chilled 1M HCl at 0° C. The mixture was extracted with DCM (3×15 mL) and combined organic layers washed with water (1×20 mL) and brine (1×10 mL) then dried over Na₂SO₄ and filtered off. The filtrate was concentrated under reduced pressure and the residue purified through flash silica gel chromatography (EtOAc in hexanes 0%-50% with 2% TEA). Yields; quantitative, colorless oils that solidifies to a white solid on freezing.

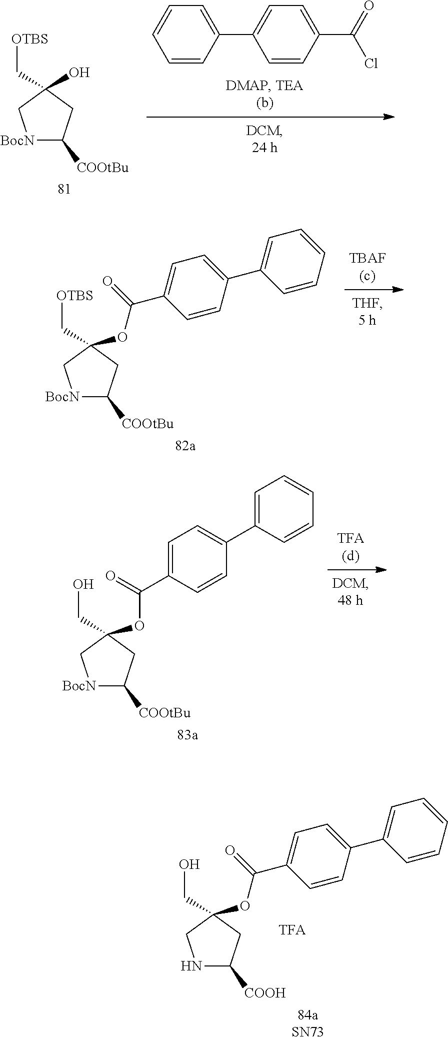

### General Procedure for Scheme 11B Step (b) and Step (c)

Crude 82a was prepared as follows; 81 (85 mg, 0.20 mmol, 1.0 eqv), DMAP (84 mg, 0.69 mmol, 3.5 eqv), biphenyl]-4-carbonyl chloride (107 mg, 0.49 mmol, 2.5 eqv) were dissolved in dry DCM (8 mL) then cooled to 0° C. TEA (1 mL, 5.91 mmol, 15.0 eqv) was added dropwise under N₂ gas and the reaction mixture left to warm up to room temperature and stirred for 24 hours. The reaction was quenched with saturated NH₄Cl (5 mL) and DCM evaporated. 20 mL of 50% EtOAc in hexanes was added and the Organic layer washed with saturated NaHCO₃ (3×10 mL) and brine/water mixture 1:1 (1×10 mL) then dried over Na₂SO₄ and filtered off. The filtrate was concentrated in vacuo and the product purified using flash silica gel chromatography (0-25% ethyl acetate in hexanes with 2% TEA)−(two columns). Product details; 97 mg, not pure and used in the next step without further purification.

83a was prepared as follows; crude 82a (97 mg, 0.16 mmol, 1.0 eq) was dissolved in THE (6 mL) and cooled to 0° C. 1M TBAF in THE (0.4 mL, 0.23 mmol, 1.5 eqv) was added dropwise under inert conditions then stirred for 2 hours. The mixture was diluted with ethyl acetate and washed with saturated NH₄Cl (2×10 mL), NaHCO₃ (3×10 mL) brine and water (1×15 mL). The organic layer was dried over Na₂SO₄, filtered off then concentrated in vacuo. The residue was purified using flash silica gel chromatography (10-80% ethyl acetate in hexanes with 2% TEA). Product details; 61 mg, 62% (2 steps) colorless oil.

84a (SN 73) was prepared according to the general procedure for scheme 11A step (iv). 83a (77 mg, 0. 16 mmol) was dissolved in dry DCM (2.5 mL) and cooled to 0° C. Trifluoroacetic acid (TFA) (2.0 mL, 26.14 mmol). Product details; 71 mg, 100% off-white powder.

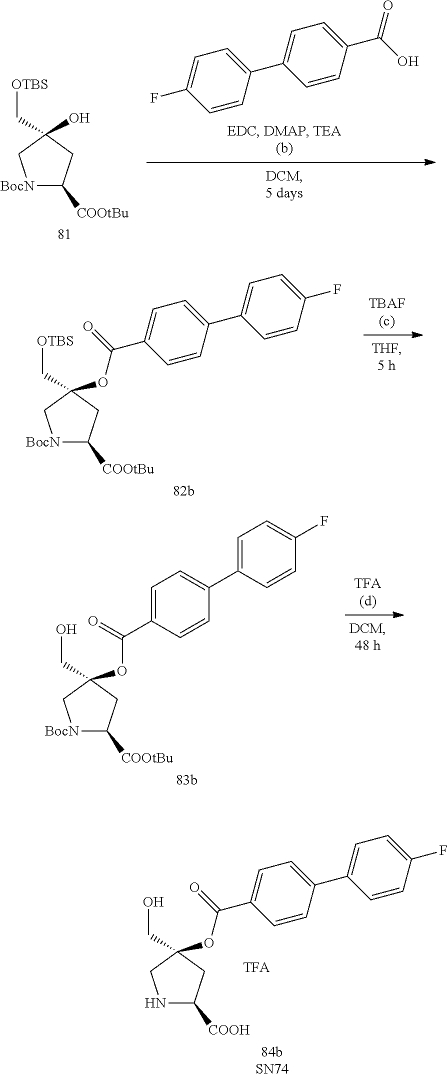

Crude 82b was prepared using Steglich esterification; 81 (38 mg, 0.09 mmol, 1.0 eq), DMAP (22 mg, 0.18 mmol, 2.0 eqv) and 4-fluorobiphenyl-4-carboxylic acid (114 mg, 0.53 mmol, 6.0 eq) were dissolved in DCM (7 mL) then cooled to 0° C. EDC (61.3 mg, 0.32 mmol, 4.0 eqv) and TEA (18 mg, 0.18 mmol) were added at 0° C. under inert conditions and reaction stirred for 5 days at room temperature. The mixture was filtered off and the filtrate concentrated in vacuo. The residue was suspended in 50% ethyl acetate in hexanes and washed with NaHCO₃ (3×15 mL), chilled 0.5M HCl (2×10 mL), brine and H₂O (1×15 mL). The organic layer was dried over anhydrous sodium sulfate and filtered off. The filtrate was concentrated under reduced pressure. The residue was purified using flash silica gel chromatography (0-15% ethyl acetate in hexanes with 2% TEA). Product not 100% pure and was used in the next step without further purification.

83b was prepared same as compound 83a; crude 82b (45 mg, 0.07 mmol) in THF (3 mL). 1M TBAF in THF (0.2 mL, 0.14 mmol). Product details; 31 mg, 69% (2 steps) colorless oil.

84b (SN 74) was prepared according to the general procedure for scheme 11A step (iv). 83b (31 mg, 0.06 mmol) was dissolved in dry DCM (1.5 mL) and cooled to 0° C. Trifluoroacetic acid (TFA) (1.0 mL, 13.07 mmol). Product details; 27 mg, 99% white solid.

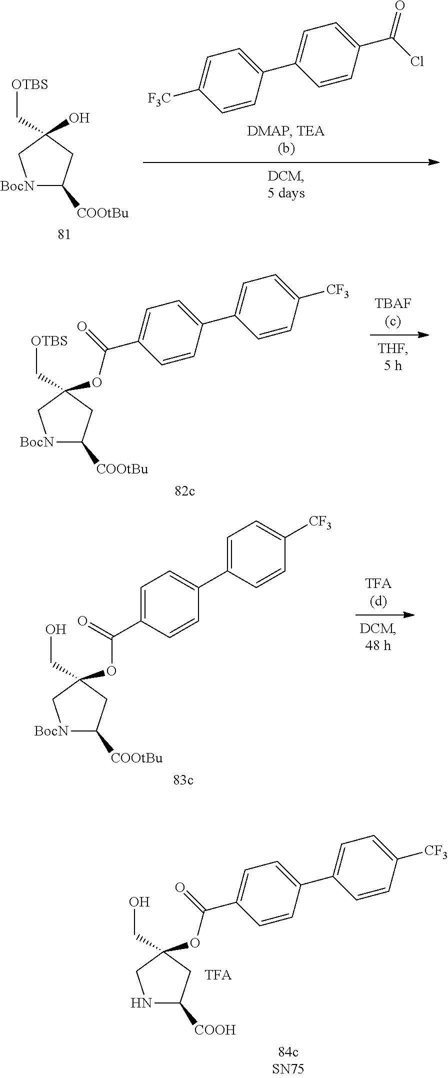

Crude 82c was prepared as crude 82a; 81 (80 mg, 0.19 mmol), DMAP (91 mg, 0.74 mmol), 4-trifluoromethylbiphenyl-4-carbonyl chloride (211 mg, 0.74 mmol) dissolved in dry DCM (8 mL). TEA used (0.4 mL, 2.78 mmol). Product not 100% pure and was used in the next step without further purification.

83c was prepared same as compound 83a; crude 82c (70 mg, 0.10 mmol) in THF (5 mL). 1M TBAF in THF (0.2 mL, 0.15 mmol). Product details; 10 mg, 10% (2 steps) colorless oil.

84c (SN 75) was prepared according to the general procedure for scheme 11A step (iv). 83c (15 mg, 0.03 mmol) was dissolved in dry DCM (1.5 mL) and cooled to 0° C. Trifluoroacetic acid (TFA) (1.0 mL, 13.07 mmol). Product details; 13 mg, 99% white solid.

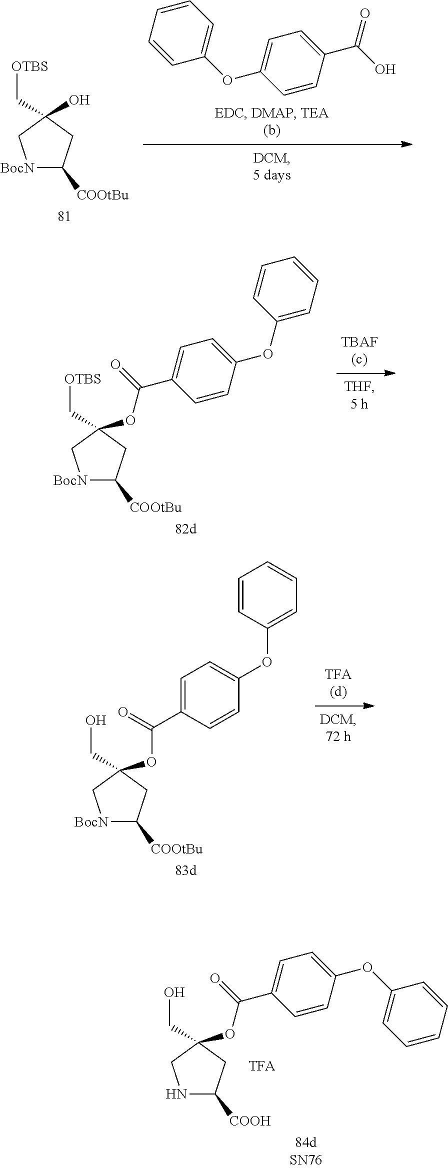

Crude 82d was prepared as crude 82b; 81 (62 mg, 0.14 mmol), DMAP (40 mg, 0.33 mmol) and 4-phenoxybenzoic acid (250 mg, 1.17 mmol) dissolved in DCM (4 mL). EDC (160 mg, 0.84 mmol) and TEA (0.2 mL, 1.44 mmol). Product details; 92 mg after column chromatography, not 100% pure and was used in the next step without further purification.

83d was prepared same as compound 83a; crude 82d (92 mg, 0.15 mmol) in THF (3 mL). 1M TBAF in THF (0.5 mL, excess). Product details; 43 mg, 58% (2 steps) colorless oil.

84d (SN 76) was prepared according to the general procedure for scheme 11A step (iv). 83d (43 mg, 0.08 mmol) was dissolved in dry DCM (3 mL) and cooled to 0° C. Trifluoroacetic acid (TFA) (1.5 mL, 13.07 mmol). Product details; 22 mg, 54% off-white solid.

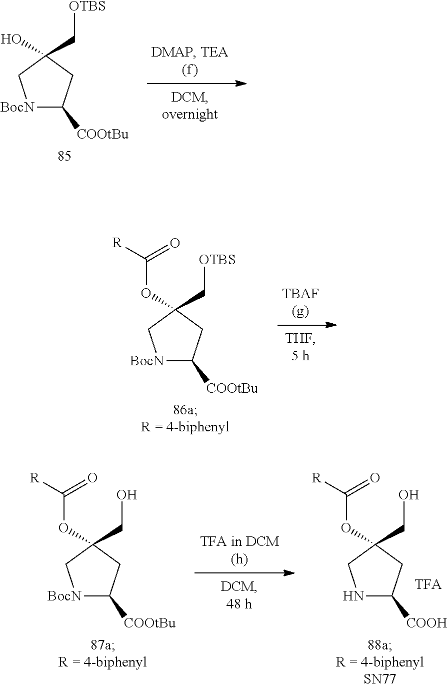

Crude 86a was prepared as crude 82a; 85 (104 mg, 0.24 mmol), DMAP (103 mg, 0.84 mmol), biphenyl-4-carbonyl chloride (131 mg, 0.60 mmol) dissolved in dry DCM (8 mL). TEA used (0.5 mL, 3.6 mmol). After column chromatography, product not 100% pure and was used in the next step without further purification.

87a was prepared same as compound 83a; crude 86a (157 mg, 0.26 mmol) in THF (6 mL). 1M TBAF in THF (0.4 mL, 0.38 mmol). Product details; 94 mg, 78% (2 steps) colorless oil.

88a (SN 77) was prepared according to the general procedure for scheme 11A step (iv). 87a (95 mg, 0.19 mmol) was dissolved in dry DCM (2 mL) and cooled to 0° C. Trifluoroacetic acid (TFA) (2.0 mL, 26.14 mmol). Product details; 81 mg, 93% light-pinkish powder.

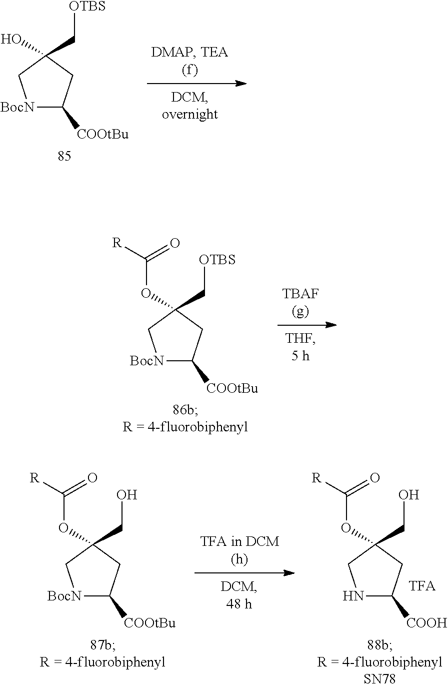

Crude 86b was prepared as crude 82a; 85 (90 mg, 0.21 mmol), DMAP (13 mg, 0.11 mmol), 4-fluorobiphenyl-4-carbonyl chloride (147 mg, 0.63 mmol) dissolved in dry DCM (3 mL). TEA used (0.1 mL, 0.42 mmol). After column chromatography, product not 100% pure and was used in the next step without further purification.

87b was prepared same as compound 83a; crude 86b (37 mg, 0.06 mmol) in THF (3 mL). 1M TBAF in THF (0.4 mL, 0.4 mmol). Product details; Not purified, pending.

88b (SN 78) to be prepared according to the general procedure for scheme 11A step (iv). 87b (1.0 eqv) dissolved in dry DCM (2 mL) and cooled to 0° C. Trifluoroacetic acid (TFA) (excess). Product details; pending.

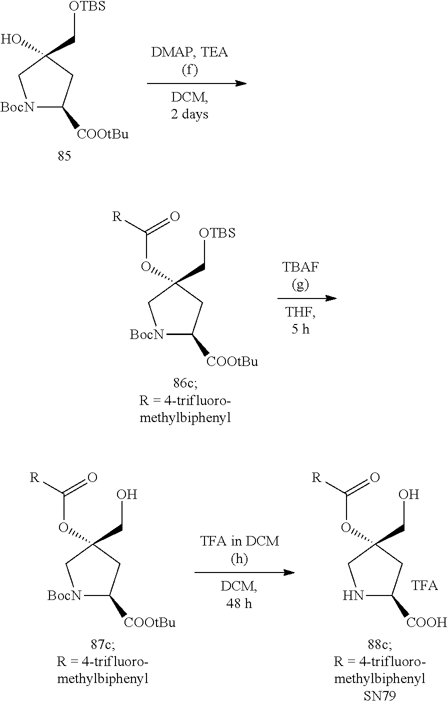

Crude 86c was prepared as crude 82a; 85 (90 mg, 0.21 mmol), DMAP (13 mg, 0.11 mmol), 4-trifluoromethylbiphenyl-4-carbonyl chloride (178 mg, 0.63 mmol) dissolved in dry DCM (3 mL). TEA used (0.1 mL, 0.42 mmol). After column chromatography, product not 100% pure and was used in the next step without further purification.

87c was prepared same as compound 83a; crude 86c (28 mg, 0.04 mmol) in THF (3 mL). 1M TBAF in THF (0.4 mL, 0.4 mmol). Product details; Not purified, pending.

88c (SN 79) to be prepared according to the general procedure for scheme 11A step (iv). 87c (1.0 eqv) dissolved in dry DCM (2 mL) and cooled to 0° C. Trifluoroacetic acid (TFA) (excess). Product details; pending.

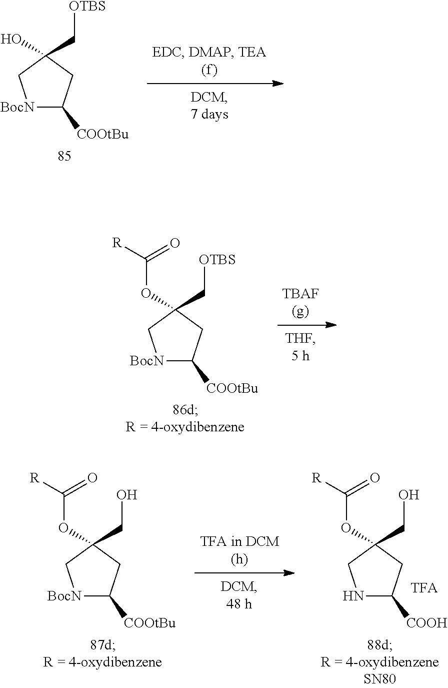

Crude 86d was prepared as crude 82b; 85 (80 mg, 0.19 mmol), DMAP (40 mg, 0.33 mmol) and 4-phenoxybenzoic acid (250 mg, 1.17 mmol) dissolved in DCM (4 mL). EDC (160 mg, 0.84 mmol) and TEA (0.2 mL, 1.44 mmol). Product details; 85 mg after column chromatography, not 100% pure and was used in the next step without further purification.

87d was prepared same as compound 83a; crude 86d (85 mg, 0.14 mmol) in THF (3 mL). 1M TBAF in THF (0.5 mL, excess). Product details; 62 mg, 65% (2 steps) clear-orange oil.

88d (SN 80) was prepared according to the general procedure for scheme 11A step (iv). 87d (62 mg, 0.12 mmol) was dissolved in dry DCM (3 mL) and cooled to 0° C. Trifluoroacetic acid (TFA) (1.5 mL, 13.07 mmol). Product details; 45 mg, 77% light-brown solid.

Cell Culture and Transfection

Human embryonic kidney 293 (HEK293, ATCC CRL-11268) cells were cultured in DMEM media supplemented with 10% (v v) fetal bovine serum (FBS), 2 mM glutamine, 1% penicillin streptomycin solution, 1 mM sodium pyruvate and non-essential amino acids. Cells were maintained at 37° C. in a fully humidified atmosphere containing 5% CO₂. rASCT2, hASCT2, hASCT1, EAAT1, EAAT2, EAAC1, EAAT5 and YFP complementary DNAs were each used to transiently transfect HEK293 using POLYPLUS Jet-prime transfection reagent. Cells were analyzed using electrophysiological techniques 24-40 hours after transfection.

Electrophysiological Techniques

Stock solutions of inhibitor was prepared in dimethyl sulfoxide (DMSO) up to 100 mM. Dilutions to working concentrations were made using external buffer. The highest DMSO concentration used (2%) did not affect electrophysiological results, as shown in control cells (data not shown). For rASCT2, hASCT2 and hASCT1, external buffer contained 140 mM NaCl, 2 mM MgCl₂, 2 mM CaCl₂), and 10 mM HEPES, pH 7.40 while internal pipette solution comprised of 130 mM NaSCN, 2 mM MgCl₂, 10 mM EGTA, 10 mM HEPES and 10 mM alanine, pH 7.40. For specificity experiments with EAAT1, EAAT2, EAAC1 and EAAT5, internal solution contained 10 mM glutamate instead of alanine. Compounds were applied to HEK293 cells expressing DNA of interest suspended from a current recording electrode in whole cell configuration through a rapid solution exchange device. Cells are immersed in external buffer bath used to dissolve the inhibitors. The open pipette resistance was between 3 and 6 MΩ. Series resistance was not compensated in these experiments due to relatively small currents. Currents traces were recorded using an Adams and List EPC7 amplifier and digitized using a Molecular Devices Digidata A/D converter.

Data Analysis

Linear and nonlinear curve fitting of the experimental data were analyzed using MicroCal Origin software. Nonlinear dose-response relationships were fitted with a Michaelis-Menten-like equation to obtain apparent Kᵢ values in the absence of substrate (see TABLE 2 for Kᵢ values). The figures show representative current traces and dose-response curves for hACST2 and rASCT2, used to obtain Kᵢ values in TABLE 2 for compound SN 40. At least four experiments were performed with at least three different cells. Error bars in all graphs, e.g., in FIG. 1B and FIG. 1C and tables (Kᵢ values) represent mean±SD.

Figures: Typical electrophysiological results used to obtain Kᵢ Values in TABLE 2. FIG. 1A Original current traces were obtained when increasing concentrations of SN 40 were applied to ASCT2 expressing HEK293 cells. FIG. 1B hASCT2 dose-response curve fitted from current traces shown in FIG. 1A. FIG. 1C shows rASCT2 dose-response fit obtained from current traces similar to that shown in FIG. 1A. All Kᵢ values shown in TABLE 2 were obtained from such dose-response curves. SN 05 was tested and is featured in TABLE 2, but in some embodiments, it is not claimed.

Results from the testing described above is shown below in TABLE 2:

TABLE 2

| | Ki (μM) | | | | | | |
|---|---|---|---|---|---|---|---|
| Ex. # | hASCT1 | rASCT2 | hASCT2 | EAAT1 | EAAT2 | EAAC1 | EAAT5 |
| SN 05 | 2.77 ± 0.1 | 0.73 ± 0.1 | 0.87 ± 0.1 | 3.70 ± 0.2 | 7.25 ± 1.5 | 7.23 ± 1.4 | 2.22 ± 0.5 |
| SN 06 | | 5.63 ± 1.0 | 5.56 ± 4.7 | | | | |
| SN 07 | X | X | X | X | X | X | X |
| SN 08 | 0.90 ± 0.1 | 0.65 ± 0.04 | 0.74 ± 0.1 | 0.40 ± 0.1 | 1.29 ± 0.3 | 0.69 ± 0.1 | 2.40 ± 0.7 |
| SN 09 | 0.44 ± 0.1 | 0.27 ± 0.05 | 1.05 ± 0.2 | 1.00 ± 0.3 | 0.97 ± 0.1 | 1.25 ± 0.2 | 1.09 ± 0.2 |
| SN 10 | 1.33 ± 0.3 | 0.97 ± 0.1 | 1.12 ± 0.1 | 1.00 ± 0.1 | 1.30 ± 0.1 | 1.99 ± 0.4 | 2.50 ± 0.9 |
| SN 11 | 8.20 ± 0.8 | 0.87 ± 0.1 | 9.13 ± 1.7 | 4.75 ± 0.3 | 3.14 ± 0.5 | 0.95 ± 0.2 | 1.17 ± 0.1 |
| SN 12 | 6.86 ± 1.1 | 6.66 ± 1.2 | 21.39 ± 3.2 | 21.2 ± 4.5 | 15.4 ± 4.9 | X | 13.81 ± 2.7 |
| SN 13 | 43.00 ± 13.4 | 18.80 ± 5.0 | 5.65 ± 1.3 | 39.46 ± 19 | X | X | X |
| SN 14 | | 4.58 ± 1.1 | | | | | |
| SN 15 | 8.51 ± 3.9 | 3.37 ± 0.4 | 2.94 ± 0.9 | 1.29 ± 0.2 | X | X | X |
| SN 16 | 10.07 ± 0.6 | 0.11 ± 0.04 | 0.39 ± 0.1 | 49.4 ± 9.7 | X | X | 566 ± 188 |
| SN 17 | 9.97 ± 0.5 | < 100 nM | < 100 nM | | | | |
| SN 18 | 7.01 ± 2.1 | 2.79 ± 0.7 | 3.01 ± 0.5 | 12.37 ± 1.0 | | | |
| SN 19 | 4.37 ± 0.8 | 0.89 ± 0.2 | 7.93 ± 0.9 | 10.20 ± 2.5 | 7.95 ± 1.1 | 14.2 ± 0.7 | |
| SN 20 | 5.71 ± 0.9 | 3.68 ± 0.7 | 4.17 ± 0.5 | | | | |
| SN 21 | >100 | 8.17 ± 1.1 | X | X | X | X | X |
| SN 22 | | 10.00 ± 5.1 | | | | | |
| SN 23 | | 20.78 ± 4.7 | | | | 14.90 ± 1.8 | |
| SN 24 | | 19.65 ± 3.1 | | | | | |
| SN 25 | 11.64 ± 1.8 | 4.40 ± 1.5 | 22.63 ± 3.7 | 9.57 ± 1.6 | 11.0 ± 1.3 | 12.26 ± 1.9 | 12.26 ± 3.2 |
| SN 26 | | 22.03 ± 4.5 | | | | | |
| SN 27 | | 5.17 ± 1.2 | | | | | |
| SN 28 | | X | | | | | |
| SN 33 | X | 3.51 ± 0.6 | 3.50 ± 0.6 | | | | |
| SN 34 | X | 3.63 ± 1.1 | 10.01 ± 2.8 | X | X | X | X |
| SN 35 | | 4.83 ± 1.0 | 0.84 ± 0.2 | X | X | X | X |
| SN 36 | X | 6.02 ± 1.8 | 6.38 ± 1.2 | X | X | X | X |
| SN 37 | | 7.46 ± 0.9 | 2.71 ± 0.5 | X | X | X | X |
| SN 38 | X | 0.38 ± 0.17 | | | | | |
| SN 39 | 2.82 ± 0.1 | 1.13 ± 0.1 | 0.98 ± 0.1 | 0.93 ± 0.1 | 2.09 ± 0.5 | 1.27 ± 0.2 | 1.41 ± 0.3 |
| SN 40 | | 7.29 ± 1.8 | 2.42 ± 0.1 | 2.94 ± 0.4 | 5.55 ± 1.7 | 24.43 ± 2.5 | 5.55 ± 0.7 |
| SN 41 | | | 1.15 ± 0.9 | | | | |
| SN 42 | 172.3 ± 24.0 | 74.7 ± 18.3 | 103.4 ± 11.2 | | | | |
| SN 43 | 20.0 ± 2.3 | 27.2 ± 1.9 | 33.6 ± 2.5 | | | | |
| SN 45 | | 1.68 ± 0.6 | 4.69 ± 1.1 | | | | |
| SN 46 | | | | | | | |
| SN 47 | 141.0 ± 17.6 | 151.5 ± 13.3 | 226.9 ± 34.2 | | | | |
| SN 48 | 7.9 ± 0.5 | 12.4 ± 1.8 | 17.3 ± 2.1 | | | | |
| SN 50 | | 1.34 ± 0.1 | 1.61 ± 0.2 | 2.71 ± 0.7 | | 20.1 ± 6.2 | |
| SN 51 | | | | | | | |
| SN 55 | | | | | | | |
| SN 56 | | | | | | | |

X indicates that the compound shows no activity.
A blank box indicates that testing has not been completed.

TABLE 3 shows additional compounds and results from the syntheses and assays described above. These examples do not limit the present invention.

TABLE 3

| | | | Kᵢ (μM) | | |
|---|---|---|---|---|---|
| | | | | | Competition Expt |
| | Dihydroxyproline | | | | (h/rASCT2 Ki) |
| | Derivatives | | | | % block (given as a |
| SN | Structure | *Stereochem | All DNA | | decimal) |
| SN65 | 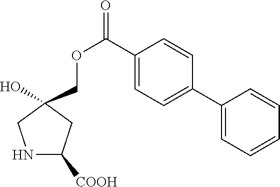 [SMILES: O=C(OC[C@]1(O)CN[C@H](C(=O)O)C1)c1ccc(-c2ccccc2)cc1] | Top Spot 1⁰ 2S, 4R | hASCT2: 30.46 ± 6.01 rASCT2: 25.76 ± 5.49 hASCT1: 59.17 ± 17.1 EAAT1 EAAT2 | | 0.5 mM ala: 52.24, 0.8 0.5 mM ala: 170, 0.4 |
| SN57 | 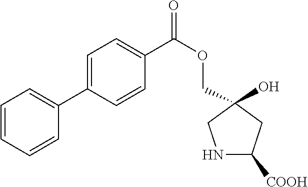 [SMILES: O=C(OC[C@@]1(O)CN[C@H](C(=O)O)C1)c1ccc(-c2ccccc2)cc1] | Bottom Spot 1⁰ 2S, 4S | hASCT2: 4.55 ± 1.22 rASCT2: 3.87 ± 0.55 hASCT1: 6.61 ± 0.47 EAAT1 EAAT2 | | 1 mM ala: 40.05, full |
| SN77 | 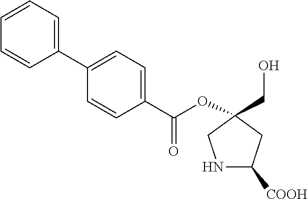 [SMILES: O=C(O[C@@]1(CO)CN[C@H](C(=O)O)C1)c1ccc(-c2ccccc2)cc1] | Top Spot 3⁰ 2S, 4R | hASCT2: NA rASCT2: NA hASCT1 EAAT1 EAAT2 | | 0.5 mM ala: 20.34, 0.5 0.5 mM ala: 12.86, 0.5 |
| SN73 | 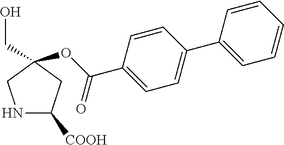 [SMILES: O=C(O[C@]1(CO)CN[C@H](C(=O)O)C1)c1ccc(-c2ccccc2)cc1] | Bottom Spot 3⁰ 2S, 4S | hASCT2: 4.25 ± 0.83 rASCT2: 8.03 ± 0.52 EAAT1: NA EAAT2: 21.45 ± 7.39 EAAC1: NA | | 1 mM ala: 22.33, full |
| SN66 | 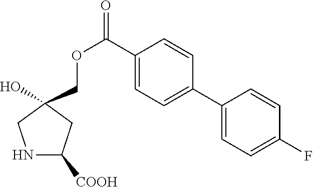 [SMILES: O=C(OC[C@]1(O)CN[C@H](C(=O)O)C1)c1ccc(-c2ccc(F)cc2)cc1] | Top Spot 1⁰ 2S, 4R | hASCT2: 15.46 ± 9.37 rASCT2: 27.63 ± 4.00 hASCT1: NC | | 1 mM ala: 28, NS 1 mM ala: 49, NS |
| SN58 | 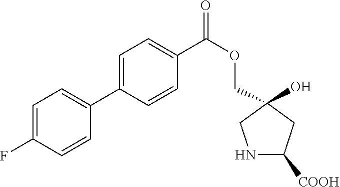 [SMILES: O=C(OC[C@@]1(O)CN[C@H](C(=O)O)C1)c1ccc(-c2ccc(F)cc2)cc1] | Bottom Spot 1⁰ 2S, 4S | hASCT2: Very Low* rASCT2: Very Low* hASCT1: 3.06 ± 0.44 | | 1 mM ala: 19.23, full 1 mM ala: 12.83, full |
| SN78 | 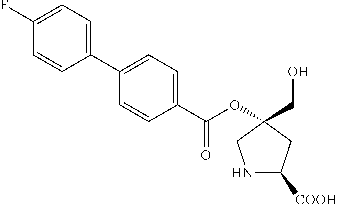 [SMILES: O=C(O[C@@]1(CO)CN[C@H](C(=O)O)C1)c1ccc(-c2ccc(F)cc2)cc1] | Top Spot 3⁰ 2S, 4R | NA | NA | NA |
| SN74 | 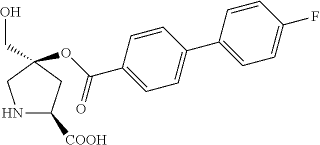 [SMILES: O=C(O[C@]1(CO)CN[C@H](C(=O)O)C1)c1ccc(-c2ccc(F)cc2)cc1] | Bottom Spot 3⁰ 2S, 4S | hASCT2: 3.20 ± 0.21 rASCT2: 2.16 ± 0.45 hASCT1: 2.45 ± 0.21 | | |
| SN67 | 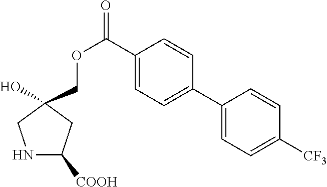 [SMILES: O=C(OC[C@]1(O)CN[C@H](C(=O)O)C1)c1ccc(-c2ccc(C(F)(F)F)cc2)cc1] | Top Spot 1⁰ 2S, 4R | hASCT2: 115 ± 89 rASCT2: NA hASCT1: NA, NC | | 0.5 mM ala: NI, 0 0.5 mM ala: NI, 0 |
| SN59 | 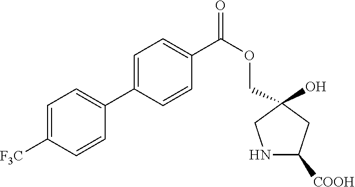 [SMILES: O=C(OC[C@@]1(O)CN[C@H](C(=O)O)C1)c1ccc(-c2ccc(C(F)(F)F)cc2)cc1] | Bottom Spot 1⁰ 2S, 4S | hASCT2: 3.24 ± 0.66 rASCT2: 0.005 hASCT1: 0.002 EAAT1: EAAT2: EAAC1: | | 1 mM ala: 19.4, full 1 mM ala: 0.15, full |
| SN79 | 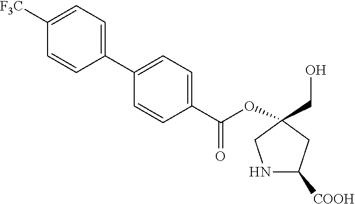 [SMILES: O=C(O[C@@]1(CO)CN[C@H](C(=O)O)C1)c1ccc(-c2ccc(C(F)(F)F)cc2)cc1] | Top Spot 3⁰ 2S, 4R | NA | NA | NA |

| | | | |
|---|---|---|---|
| SN75 | 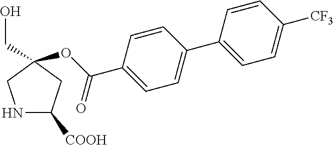 [SMILES: O=C(O[C@]1(CO)CN[C@H](C(=O)O)C1)c1ccc(-c2ccc(C(F)(F)F)cc2)cc1] | Bottom Spot 3⁰ 2S, 4S | hASCT2: 8.07 ± 0.75 rASCT2: 3.31 ± 0.51 hASCT1: 1.17 ± 0.31 |
| SN69 | 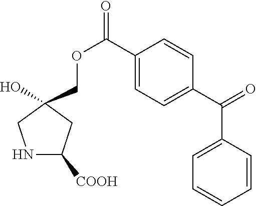 [SMILES: O=C(OC[C@]1(O)CN[C@H](C(=O)O)C1)c1ccc(C(=O)c2ccccc2)cc1] | Top Spot 1⁰ 2S, 4R | |
| SN61 | 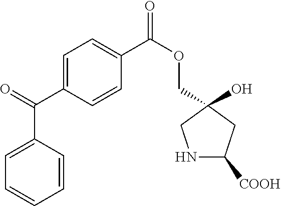 [SMILES: O=C(OC[C@@]1(O)CN[C@H](C(=O)O)C1)c1ccc(C(=O)c2ccccc2)cc1] | Bottom Spot 1⁰ 2S, 4S | hASCT2: 108 ± 12 rASCT2: 88.6 ± 9.3 hASCT1: 18.6 ± 6.55 |
| SN71 | 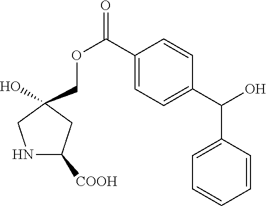 [SMILES: O=C(OC[C@]1(O)CN[C@H](C(=O)O)C1)c1ccc(C(O)c2ccccc2)cc1] | Top Spot 1⁰ 2S, 4R | |
| SN63 | 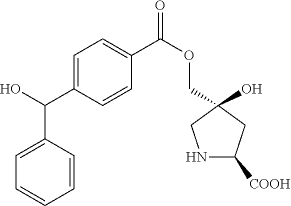 [SMILES: O=C(OC[C@@]1(O)CN[C@H](C(=O)O)C1)c1ccc(C(O)c2ccccc2)cc1] | Bottom Spot 1⁰ 2S, 4S | hASCT2: 405 ± 119 rASCT2: 232 ± 78 hASCT1: 93 ± 35 |
| SN68 | 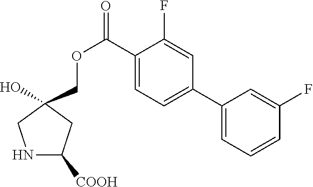 [SMILES: O=C(OC[C@]1(O)CN[C@H](C(=O)O)C1)c1ccc(-c2cccc(F)c2)cc1F] | Top Spot 1⁰ 2S, 4R | |
| SN60 | 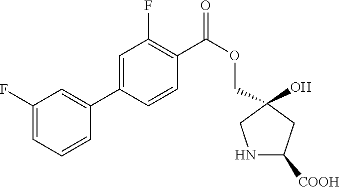 [SMILES: O=C(OC[C@@]1(O)CN[C@H](C(=O)O)C1)c1ccc(-c2cccc(F)c2)cc1F] | Bottom Spot 1⁰ 2S, 4S | hASCT2: 4.61 ± 0.64 rASCT2: 4.87 ± 0.78 hASCT1: 8.15 ± 1.01 |
| SN70 | 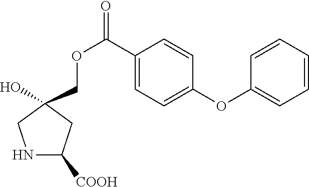 [SMILES: O=C(OC[C@]1(O)CN[C@H](C(=O)O)C1)c1ccc(Oc2ccccc2)cc1] | Top Spot 1⁰ 2S, 4R | |
| SN62 | 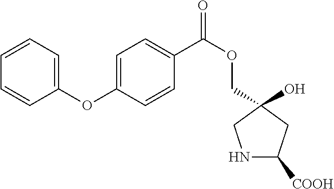 [SMILES: O=C(OC[C@@]1(O)CN[C@H](C(=O)O)C1)c1ccc(Oc2ccccc2)cc1] | Bottom Spot 1⁰ 2S, 4S | |
| SN80 | 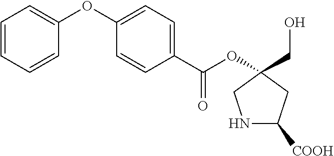 [SMILES: O=C(O[C@@]1(CO)CN[C@H](C(=O)O)C1)c1ccc(Oc2ccccc2)cc1] | Top Spot 3⁰ 2S, 4R | |
| SN76 | 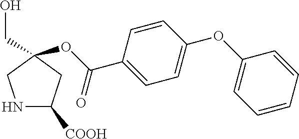 [SMILES: O=C(O[C@]1(CO)CN[C@H](C(=O)O)C1)c1ccc(Oc2ccccc2)cc1] | Bottom Spot 3⁰ 2S, 4S | hASCT2: 31.87 ± 549 rASCT2: 20.01 ± 1.75 hASCT1: 12.20 ± 12 |
| SN72 | 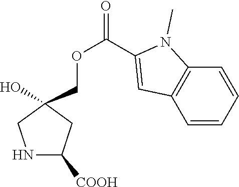 [SMILES: Cn1c(C(=O)OC[C@]2(O)CN[C@H](C(=O)O)C2)cc2ccccc21] | Top Spot 1⁰ 2S, 4R | |
| SN64 | 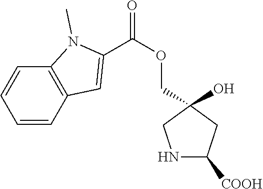 [SMILES: Cn1c(C(=O)OC[C@@]2(O)CN[C@H](C(=O)O)C2)cc2ccccc21] | Bottom Spot 1⁰ 2S, 4S | |

*Top spot and Bottom spot indicates a compound made from a diol whose RF on TLC was 0.50 and 0.46 respectively using a 1:1 EtOAc/hexanes system. Stereochemistry was predicted by Sharpless asymmetric dihydroxylation reaction using regioselective AD-mix-a and AD-mix-b reagents.
NA: Synthesis pending (1 step)

While typical embodiments have been set forth for the purpose of illustration, the foregoing descriptions and examples should not be deemed to be a limitation on the scope of the invention. Accordingly, various modifications, adaptations, and alternatives may occur to one skilled in the art without departing from the spirit and scope of the present invention.

We claim:

1. A compound of formula I: 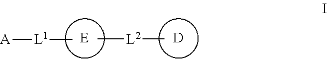
wherein:
A is selected from: 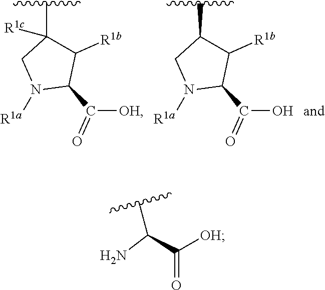
R^{1a} is selected from —H and —(C₁-C₆)alkyl;
R^{1b} is selected from —H, —OH, —CON(H)₂, and —NHC(═O)(H);
R^{1c} is —(CH₂)ₚOH—;
L¹ is selected from —(CHR²)ₙOR³—, —R³O(CHR²)ₙ—, —(CH₂)ₘN(R⁴)R³—, and —R³(R⁴)N(CH₂)ₘ—;
m is selected independently in each instance from 0 and 1;
n is selected from 0, 1, and 2;
p is selected from 0, 1, 2, and 3;
R² is selected independently in each instance from —H, —(CH₂)ₘOH, and —CH_{3;}
R³ is selected from —C(═O)— and —CH₂—;
R⁴ is selected independently in each instance from —H and —(C₁-C₆)alkyl;
L² is selected from a direct bond, —C(═O)—, —CH(OH)—, —NH(C═O)—, —(C═O)NH—, and —(CH₂)ₘO—(CH₂)ₘ—, or is absent when [symbol] is absent; [symbol]
an aryl or heteroaryl moiety, optionally substituted with one or more substituents R⁵; [symbol]
is an aryl or heteroaryl ring, optionally substituted with one or more substituents R⁵, or is absent when [symbol] is a bicyclic or polycyclic moiety; and
R⁵ is selected independently in each instance from hydrogen, (C₁-C₆)alkyl, halogen, halo(C₁-C₆)alkyl, nitro, —OH, (C₁-C₆)alkoxy, halo(C₁-C₆)alkoxy, cyano, (C₁-C₆)alkylthio, halo(C₁-C₆)alkylthio, and amino;
with the proviso that both of R^{1c} and L¹ are not connected to A by an oxygen or a nitrogen atom; and
with the proviso that the following compounds are excluded:

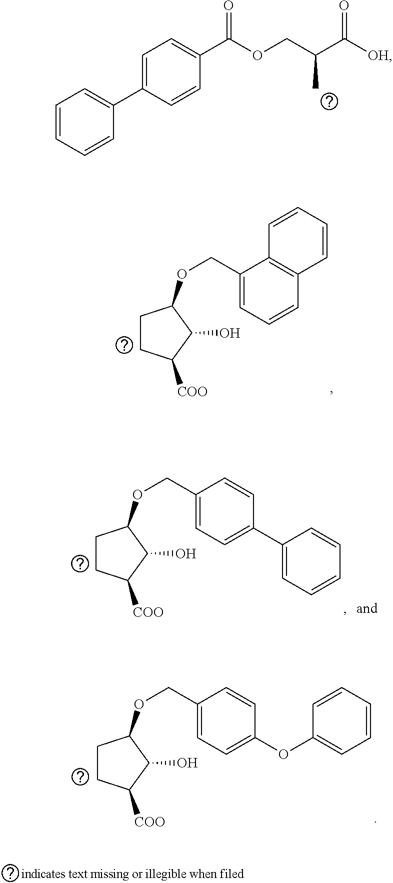

2. A compound of formula I according to claim 1: 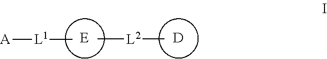
wherein:
A is selected from: 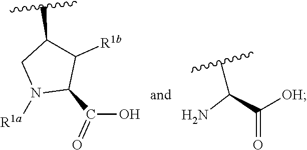
R^{1a} is selected from —H and —(C₁-C₆)alkyl;
R^{1b} is selected from —H, —OH, —CON(H)₂, and —NHC(═O)(H);
L¹ is selected from —(CHR²)ₙOR³—, —R³O(CHR²)ₙ—, —(CH₂)ₘN(R⁴)R³—, and —R³(R⁴)N(CH₂)ₘ—;
m is selected independently in each instance from 0 and 1;
n is selected from 0, 1, and 2;
R² is selected independently in each instance from —H, —(CH₂)ₘOH, and —CH₃;
R³ is selected from —C(═O)— and —CH₂—;
R⁴ is selected independently in each instance from —H and —(C₁-C₆)alkyl;
L² is selected from a direct bond, —C(═O)—, —CH(OH)—, —NH(C═O)—, —(C═O)NH—, and —(CH₂)ₘO—(CH₂)ₘ—, or is absent when [symbol] is absent; [symbol]
is an aryl or heteroaryl moiety, optionally substituted with one or more substituents R⁵; [symbol]
is an aryl or heteroaryl ring, optionally substituted with one or more substituents R⁵, or is absent when [symbol] is a bicyclic or polycyclic moiety; and
R⁵ is selected independently in each instance from hydrogen, (C₁-C₆)alkyl, halogen, halo(C₁-C₆)alkyl, nitro, —OH, (C₁-C₆)alkoxy, halo(C₁-C₆)alkoxy, cyano, (C₁-C₆)alkylthio, halo(C₁-C₆)alkylthio, and amino;
with the proviso that the following compounds are excluded:

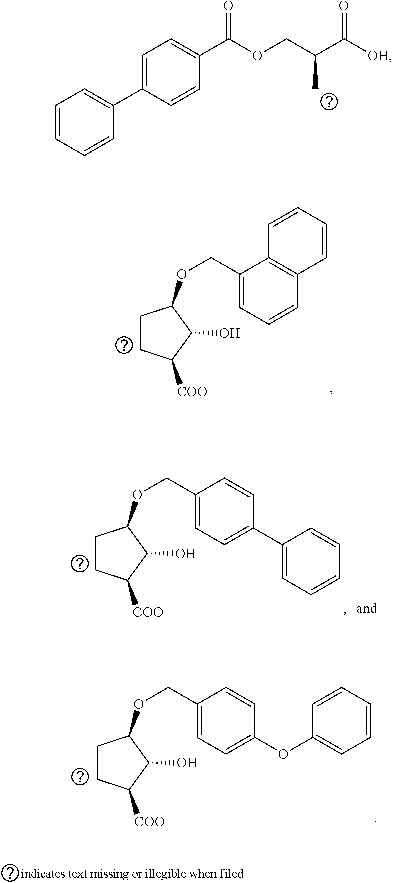

3. The compound according to claim 1, wherein L¹ is selected from —(CHR²)ₙOR³—, —OC(═O)—, —(CH₂)ₘN(R⁴)R³—, —NHC(═O)—, and —NH(CH₂)—; or wherein [symbol] is selected from optionally substituted phenyl, thiophene, pyridine, anthracene, pyrimidine, furan, indole, and naphthalene.

4. (canceled)

5. The compound according to claim 1, wherein [symbol] is selected from optionally substituted phenyl, thiophene, pyridine, pyrimidine and furan, optionally wherein L² is selected from a direct bond and —NH(C═O)—.

6. (canceled)

7. The compound according to claim 1, wherein 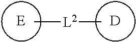
is selected from

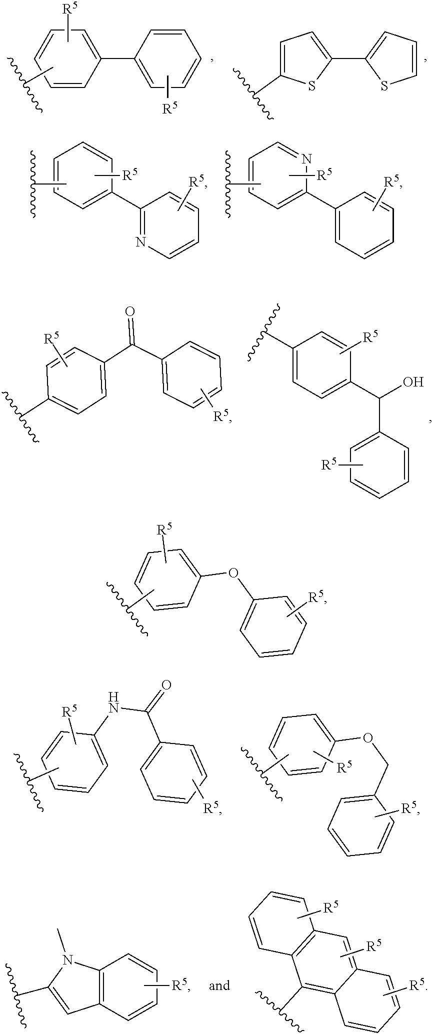

8. The compound according to claim 7, wherein

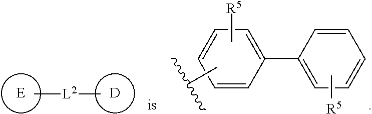

9. The compound according to claim 7, wherein

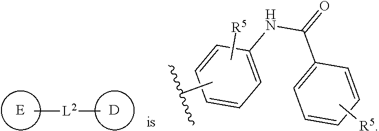

10. The compound according to claim 1, wherein R⁵ is selected independently in each instance from hydrogen, methyl, fluoro, —CF₃, —CH₃Br, nitro, —OH, and methoxy.

11-12. (canceled)

13. The compound according to claim 1, wherein A is

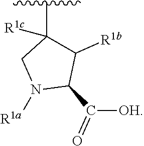

14. The compound according to claim 13, wherein A is

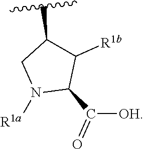

15. The compound according to claim 13, wherein R^{1a} and R^{1b} are both —H, or wherein R^{1c} is —OH or —(CH₂)OH.

16. (canceled)

17. The compound according to claim 1,
wherein A is

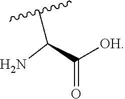 [SMILES: *[C@H](N)C(=O)O]

18. The compound according to claim 1 of formula IIa′, formula IIa or IIb: 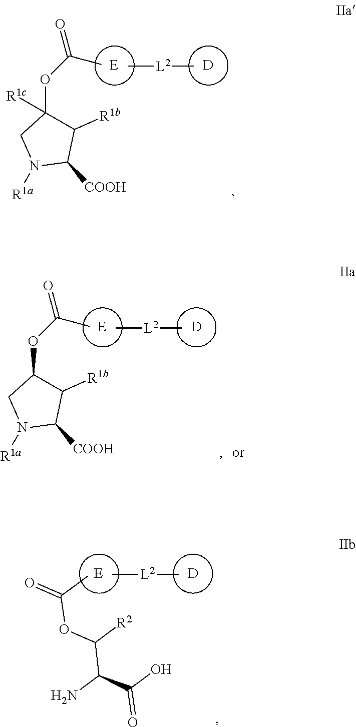
optionally wherein R² is —H or —OH.

19. (canceled)

20. The compound according to claim 1 of formula IIIa′, formula IIIa, formula IIIb, formula IVa′, formula IVa, formula Va, or formula Vb:

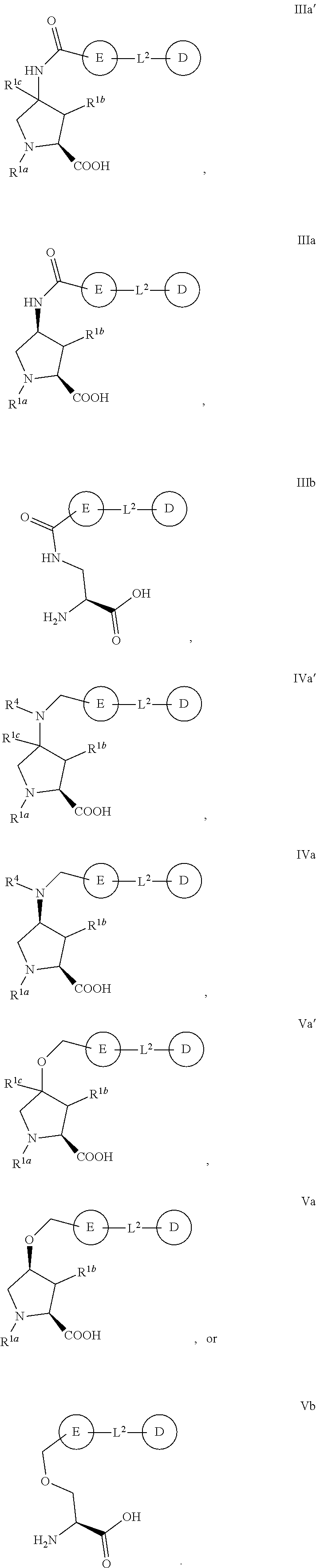

21. The compound according to claim 1, selected from a compound of Table 1.

22. A pharmaceutical composition comprising a pharmaceutically acceptable carrier and a compound according to claim 1.

23. A method for treating cancer in a patient in need thereof comprising administering an effective dose of a compound according to claim 1, preferably wherein the cancer is selected from breast cancer, prostate cancer, and melanoma.

24. A method for treating a disease or disorder in a patient wherein the disease or disorder involves the dysregulation of ASCT2, the method comprising administering to the patient a therapeutically effective amount of a compound according to claim 1.

25. A method for inhibiting ASCT2, said method comprising bringing ASCT2 into contact with a compound according to claim 1, wherein the method is selected from in vitro or in vivo.

26. A compound selective for an ASCT transporter, wherein said compound interacts with the ASCT transporter, but interacts with less potency with an EAAT transporter, wherein said compound is a compound according to claim 1.
